(19) United States
(12) Patent Application Publication
(10) Pub. No.: US 2008/0280910 A1
(43) Pub. Date: Nov. 13, 2008

(54) PHTHALAZINONE DERIVATIVES

(76) Inventors: Keith Allan Menear, Cambridge (GB); Marc Geoffrey Hummersone, Cambridge (GB); Sylvie Gomez, Cambridge (GB); Muhammad Hashim Javaid, Cambridge (GB); Niall Morrison Barr Martin, Cambridge (GB); Frank Kerrigan, Cornwall (GB)

Correspondence Address: MICHAEL BEST & FRIEDRICH LLP, ONE SOUTH PINCKNEY STREET, P O BOX 1806, MADISON, WI, 53701, US

(21) Appl. No.: 12/051,219

(22) Filed: Mar. 19, 2008

Related U.S. Application Data
(60) Provisional application No. 60/896,340, filed on Mar. 22, 2007.

(51) Int. Cl. A61K 31/502; C07D 237/32; A61P 35/04

(52) U.S. Cl. 514/248; 544/237

(57) ABSTRACT

A compound of the formula (I):

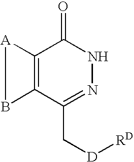

wherein:
A and B together represent an optionally substituted, fused aromatic ring or an optionally substituted, fused cyclohexene ring;
D is selected from:

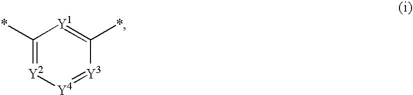

where Y¹ is selected from CH and N, Y² is selected from CH and N, Y³ is selected from CH, CF and N and Y⁴ is selected from CH and N;

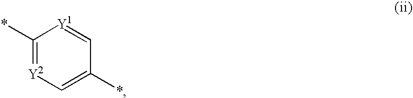

where Y¹ is selected from CH and N and Y² is selected from CH and N;

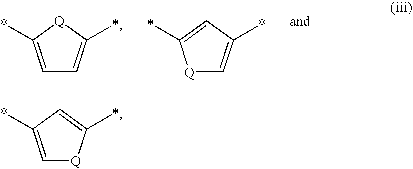

where Q is O or S; and

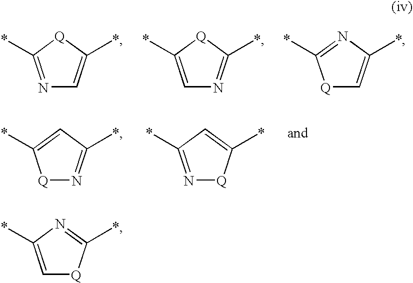

where Q is O or S; and
R^{D} is an optionally substituted C₅₋₂₀ aryl group, bound to D by a carbon-carbon bond.

This application claims priority to U.S. Provisional Application No. 60/896,340, filed Mar. 22, 2007, which is incorporated herein by reference in its entirety.

The present invention relates to phthalazinone derivatives and their use as pharmaceuticals. In particular, the present invention relates to the use of these compounds to inhibit the activity of the enzyme poly (ADP-ribose)polymerase-1, also known as poly(ADP-ribose)synthase and poly ADP-ribosyltransferase, and commonly referred to as PARP-1.

The mammalian enzyme PARP-1 (a 113-kDa multidomain protein) has been implicated in the signalling of DNA damage through its ability to recognize and rapidly bind to DNA single or double strand breaks (D'Amours, et al., Biochem. J., 342, 249-268 (1999)).

The family of Poly (ADP-ribose) polymerases now includes around 18 proteins, that all display a certain level of homology in their catalytic domain but differ in their cellular functions (Ame et al., Bioessays., 26(8), 882-893 (2004)). Of this family PARP-1 (the founding member) and PARP-2 are so far the sole enzymes whose catalytic activity are stimulated by the occurrence of DNA strand breaks, making them unique in the family.

It is now known that PARP-1 participates in a variety of DNA-related functions including gene amplification, cell division, differentiation, apoptosis, DNA base excision repair as well as effects on telomere length and chromosome stability (d'Adda di Fagagna, et al., Nature Gen., 23(1), 76-80 (1999)).

Studies on the mechanism by which PARP-1 modulates DNA repair and other processes has identified its importance in the formation of poly (ADP-ribose) chains within the cellular nucleus (Althaus, F. R. and Richter, C., ADP-Ribosylation of Proteins: Enzymology and Biological Significance, Springer-Verlag, Berlin (1987)). The DNA-bound, activated PARP-1 utilizes NAD⁺ to synthesize poly (ADP-ribose) on a variety of nuclear target proteins, including topoisomerases, histones and PARP itself (Rhun, et al., Biochem. Biophys. Res. Commun., 245, 1-10 (1998))

Poly (ADP-ribosyl)ation has also been associated with malignant transformation. For example, PARP-1 activity is higher in the isolated nuclei of SV40-transformed fibroblasts, while both leukaemic and colon cancer cells show higher enzyme activity than the equivalent normal leukocytes and colon mucosa (Miwa, et al., Arch. Biochem. Biophys., 181, 313-321 (1977); Burzio, et al., Proc. Soc. Exp. Biol. Med., 149, 933-938 (1975); and Hirai, et al., Cancer Res., 43, 3441-3446 (1983)). More recently in malignant prostate tumours compared to benign prostate cells significantly increased levels of active PARP (predominantly PARP-1) have been identified associated with higher levels of genetic instability (McNealy, et al., Anticancer Res., 23, 1473-1478 (2003)).

A number of low-molecular-weight inhibitors of PARP-1 have been used to elucidate the functional role of poly (ADP-ribosyl)ation in DNA repair. In cells treated with alkylating agents, the inhibition of PARP leads to a marked increase in DNA-strand breakage and cell killing (Durkacz, et al., Nature, 283, 593-596 (1980); Berger, N. A., Radiation Research, 101, 4-14 (1985)).

Subsequently, such inhibitors have been shown to enhance the effects of radiation response by suppressing the repair of potentially lethal damage (Ben-Hur, et al., British Journal of Cancer, 49 (Suppl. VI), 34-42 (1984); Schlicker, et al., Int. J. Radiat. Biol., 75, 91-100 (1999)). PARP inhibitors have been reported to be effective in radio sensitising hypoxic tumour cells (U.S. Pat. No. 5,032,617; U.S. Pat. No. 5,215,738 and U.S. Pat. No. 5,041,653). In certain tumour cell lines, chemical inhibition of PARP-1 (and PARP-2) activity is also associated with marked sensitisation to very low doses of radiation (Chalmers, Clin. Oncol., 16(1), 29-39 (2004))

Furthermore, PARP-1 knockout (PARP −/−) animals exhibit genomic instability in response to alkylating agents and γ-irradiation (Wang, et al., Genes Dev., 9, 509-520 (1995); Menissier de Murcia, et al., Proc. Natl. Acad. Sci. USA, 94, 7303-7307 (1997)). More recent data indicates that PARP-1 and PARP-2 possess both overlapping and non-redundant functions in the maintenance of genomic stability, making them both interesting targets (Menissier de Murcia, et al., EMBO. J., 22(9), 2255-2263 (2003)).

PARP inhibition has also recently been reported to have antiangiogenic effects. Where dose dependent reductions of VEGF and basic-fibroblast growth factor (bFGF)-induced proliferation, migration and tube formation in HUVECS has been reported (Rajesh, et al., Biochem. Biophys. Res. Comm., 350, 1056-1062 (2006)).

A role for PARP-1 has also been demonstrated in certain vascular diseases, septic shock, ischaemic injury and neurotoxicity (Cantoni, et al., Biochim. Biophys. Acta, 1014, 1-7 (1989); Szabo, et al., J. Clin. Invest., 100, 723-735 (1997)). Oxygen radical DNA damage that leads to strand breaks in DNA, which are subsequently recognised by PARP-1, is a major contributing factor to such disease states as shown by PARP-1 inhibitor studies (Cosi, et al., J. Neurosci. Res., 39, 38-46 (1994); Said, et al., Proc. Natl. Acad. Sci. U.S.A., 93, 4688-4692 (1996)). More recently, PARP has been demonstrated to play a role in the pathogenesis of haemorrhagic shock (Liaudet, et al., Proc. Natl. Acad. Sci. U.S.A., 97(3), 10203-10208 (2000)), eye (Occular) related oxidative damage as in Macular Degeneration (AMD) and retinitis pigmentosis (Paquet-Durand et al., J. Neuroscience, 27(38), 10311-10319 (2007), as well as in transplant rejection of organs like lung, heart and kidney (O'Valle, et al., Transplant. Proc., 39(7), 2099-2101 (2007). Moreover, treatment with PARP inhibitors has been shown to attenuate acute diseases like pancreatitis and it associated liver and lung damage caused by mechanisms where PARP plays a role (Mota, et al., Br. J. Pharmacol., 151(7), 998-1005 (2007).

It has also been demonstrated that efficient retroviral infection of mammalian cells is blocked by the inhibition of PARP-1 activity. Such inhibition of recombinant retroviral vector infections was shown to occur in various different cell types (Gaken, et al., J. Virology, 70(6), 3992-4000 (1996)). Inhibitors of PARP-1 have thus been developed for the use in anti-viral therapies and in cancer treatment (WO 91/18591).

Moreover, PARP-1 inhibition has been speculated to delay the onset of aging characteristics in human fibroblasts (Rattan and Clark, Biochem. Biophys. Res. Comm., 201(2), 665-672 (1994)) and age related diseases such as atherosclerosis (Hans, et al., Cardiovasc. Res., (Jan. 31, 2008)). This may be related to the role that PARP plays in controlling telomere function (d×Adda di Fagagna, et al., Nature Gen., 23(1), 76-80 (1999)).

PARP inhibitors are also thought to be relevant to the treatment of inflammatory bowel disease (Szabo C., Role of Poly(ADP-Ribose) Polymerase Activation in the Pathogenesis of Shock and Inflammation, In PARP as a Therapeutic Target; Ed J. Zhang, 2002 by CRC Press; 169-204), ulcerative colitis (Zingarelli, B, et al., Immunology, 113(4), 509-517 (2004)) and Crohn's disease (Jijon, H. B., et al., Am. J. Physiol. Gastrointest. Liver Physiol., 279, G641-G651 (2000).

Some of the present inventors have previously described (WO 02/36576) a class of 1(2H)-phthalazinone compounds which act as PARP inhibitors. The compounds have the general formula:

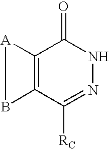

where A and B together represent an optionally substituted, fused aromatic ring and where R_{C} is represented by -L-R_{L}. A large number of examples are of the formula:

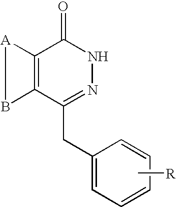

where R represent one or more optional substituents.

The present inventors have now discovered that compounds which to some extent fall within the general definition in the above mentioned application display properties of interest. Some of the compounds may exhibit surprising levels of inhibition of the activity of PARP, and/or of potentiation of tumour cells to radiotherapy and various chemotherapies. Some of the compounds of the present invention also show good solubility in both aqueous media and phosphate buffer solution—enhanced solubility may be of use in formulation the compounds for administration by an IV route, or for oral formulations (e.g. liquid and small tablet forms) for paediatric use. The oral bioavailablity of the compounds of the present invention may be enhanced.

Accordingly, the first aspect of the present invention provides a compound of the formula (I):

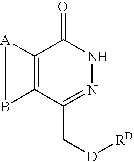

(including isomers, salts, solvates, chemically protected forms, and prodrugs thereof) wherein:
A and B together represent an optionally substituted, fused aromatic ring or an optionally substituted, fused cyclohexene ring;
D is selected from:

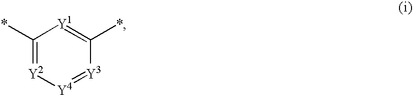

where Y¹ is selected from CH and N, Y² is selected from CH and N, Y³ is selected from CH, CF and N and Y⁴ is selected from CH and N;

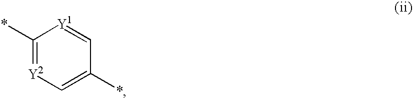

where Y¹ is selected from CH and N and Y² is selected from CH and N;

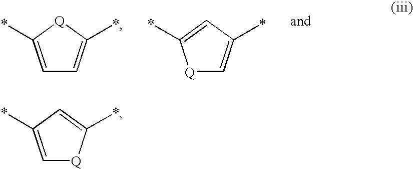

where Q is O or S; and

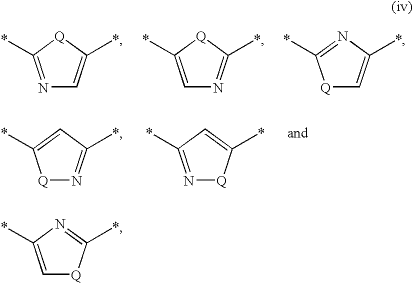

where Q is O or S; and
R^{D} is an optionally substituted C₅₋₂₀ aryl group, bound to D by a carbon-carbon bond.

The possibilities for D are:

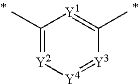

| Y¹ | Y² | Y³ | Y⁴ | |
|---|---|---|---|---|
| CH | CH | CH | CH | 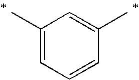 |
| CH | CH | CF | CH | 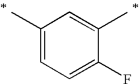 |
| N | CH | CH | CH | 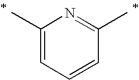 |
| N | CH | CF | CH | 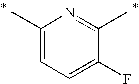 |
| CH | N | CH | CH | 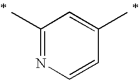 |
| CH | N | CF | CH | 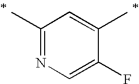 |
| CH | CH | N | CH | 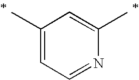 |
| N | N | CH | CH | 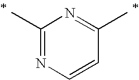 |
| N | N | CF | CH | 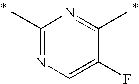 |
| N | CH | N | CH | 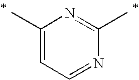 |
| CH | N | N | CH | 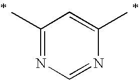 |
| N | N | N | CH | 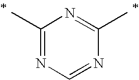 |
| CH | CH | CH | N | 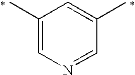 |
| CH | CH | CF | N | 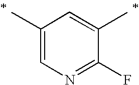 |
| N | CH | CH | N | 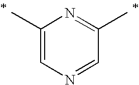 |
| N | CH | CF | N | 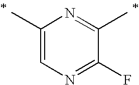 |
| CH | N | CH | N | 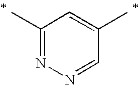 |
| CH | N | CF | N | 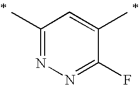 |
| CH | CH | N | N | 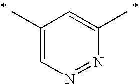 |
| N | N | CH | N | 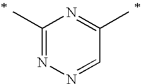 |
| N | N | CF | N | 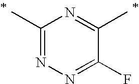 |
| N | CH | N | N | 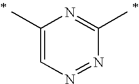 |
| CH | N | N | N | 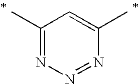 |
| N | N | N | N | 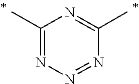 |

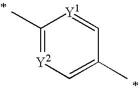

Formula

| Y¹ | Y² | Group |
|---|---|---|
| CH | CH | 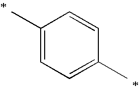 |
| N | CH | 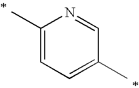 |
| CH | N | 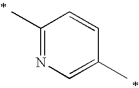 |
| N | N | 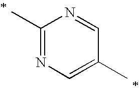 |

| | | |
|---|---|---|
| 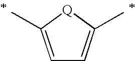 | 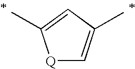 | 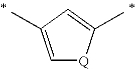 |

| Q | | | |
|---|---|---|---|
| O | 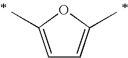 | 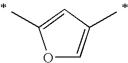 | 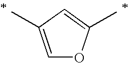 |
| S | 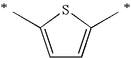 | 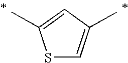 | 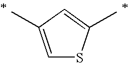 |

| | | |
|---|---|---|
| 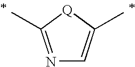 | 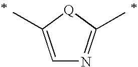 | 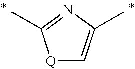 |
| 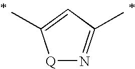 | 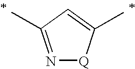 | 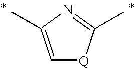 |

| Q | | | |
|---|---|---|---|
| O | 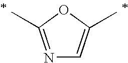 | 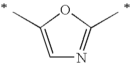 | 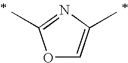 |
| | 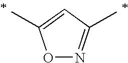 | 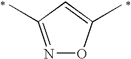 | 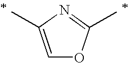 |
| S | 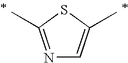 | 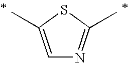 | 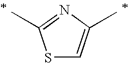 |
| | 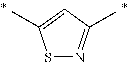 | 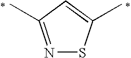 | 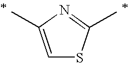 |

A second aspect of the present invention provides a pharmaceutical composition comprising a compound of the first aspect and a pharmaceutically acceptable carrier or diluent.

A third aspect of the present invention provides the use of a compound of the first aspect in a method of treatment of the human or animal body.

A fourth aspect of the present invention provides the use of a compound as defined in the first aspect of the invention in the preparation of a medicament for:

(a) preventing poly(ADP-ribose) chain formation by inhibiting the activity of cellular PARP (PARP-1 and/or PARP-2);
(b) the treatment of: vascular disease; septic shock; ischemic injury, both cerebral and cardiovascular; reperfusion injury, both cerebral and cardiovascular; neurotoxicity, including acute and chronic treatments for stroke and Parkinson's disease; haemorrhagic shock; eye related oxidative damage; transplant rejection; inflammatory diseases, such as arthritis, inflammatory bowel disease, ulcerative colitis and Crohn's disease; multiple sclerosis; secondary effects of diabetes; as well as the acute treatment of cytoxicity following cardiovascular surgery; pancreatitis; atherosclerosis; or diseases ameliorated by the inhibition of the activity of PARP;
(c) use as an adjunct in cancer therapy or for potentiating tumour cells for treatment with ionizing radiation or chemotherapeutic agents.

In particular, compounds as defined in the first aspect of the invention can be used in anti-cancer combination therapies (or as adjuncts) along with alkylating agents, such as methyl methanesulfonate (MMS), temozolomide and dacarbazine (DTIC), also with topoisomerase-1 inhibitors like Topotecan, Irinotecan, Rubitecan, Exatecan, Lurtotecan, Gimetecan, Diflomotecan (homocamptothecins); as well as 7-substituted non-silatecans; the 7-silyl camptothecins, BNP 1350; and non-camptothecin topoisomerase-I inhibitors such as indolocarbazoles also dual topoisomerase-I and II inhibitors like the benzophenazines, XR 11576/MLN 576 and benzopyridoindoles. Such combinations could be given, for example, as intravenous preparations or by oral administration as dependent on the preferred method of administration for the particular agent.

Other further aspects of the invention provide for the treatment of disease ameliorated by the inhibition of PARP, comprising administering to a subject in need of treatment a therapeutically-effective amount of a compound as defined in the first aspect, preferably in the form of a pharmaceutical composition and the treatment of cancer, comprising administering to a subject in need of treatment a therapeutically-effective amount of a compound as defined in the first aspect in combination, preferably in the form of a pharmaceutical composition, simultaneously or sequentially with radiotherapy (ionizing radiation) or chemotherapeutic agents.

In further aspects of the present invention, the compounds may be used in the preparation of a medicament for the treatment of cancer which is deficient in Homologous Recombination (HR) dependent DNA double strand break (DSB) repair activity, or in the treatment of a patient with a cancer which is deficient in HR dependent DNA DSB repair activity, comprising administering to said patient a therapeutically-effective amount of the compound.

The HR dependent DNA DSB repair pathway repairs double-strand breaks (DSBs) in DNA via homologous mechanisms to reform a continuous DNA helix (K. K. Khanna and S. P. Jackson, Nat. Genet. 27(3): 247-254 (2001)). The components of the HR dependent DNA DSB repair pathway include, but are not limited to, ATM (NM_{—}000051), RAD51 (NM_{—}002875), RAD51L1 (NM_{—}002877), RAD51C (NM_{—}002876), RAD51L3 (NM_{—}002878), DMC1 (NM_{—}007068), XRCC2 (NM_{—}005431), XRCC3 (NM_{—}005432), RAD52 (NM_{—}002879), RAD54L (NM_{—}003579), RAD54B (NM_{—}012415), BRCA1 (NM_{—}007295), BRCA2 (NM_{—}000059), RAD50 (NM_{—}005732), MRE11A (NM_{—}005590) and NBS1 (NM_{—}002485). Other proteins involved in the HR dependent DNA DSB repair pathway include regulatory factors such as EMSY (Hughes-Davies, et al., Cell, 115, pp 523-535). HR components are also described in Wood, et al., Science, 291, 1284-1289 (2001).

A cancer which is deficient in HR dependent DNA DSB repair may comprise or consist of one or more cancer cells which have a reduced or abrogated ability to repair DNA DSBs through that pathway, relative to normal cells i.e. the activity of the HR dependent DNA DSB repair pathway may be reduced or abolished in the one or more cancer cells.

The activity of one or more components of the HR dependent DNA DSB repair pathway may be abolished in the one or more cancer cells of an individual having a cancer which is deficient in HR dependent DNA DSB repair. Components of the HR dependent DNA DSB repair pathway are well characterised in the art (see for example, Wood, et al., Science, 291, 1284-1289 (2001)) and include the components listed above.

In some preferred embodiments, the cancer cells may have a BRCA1 and/or a BRCA2 deficient phenotype i.e. BRCA1 and/or BRCA2 activity is reduced or abolished in the cancer cells. Cancer cells with this phenotype may be deficient in BRCA1 and/or BRCA2, i.e. expression and/or activity of BRCA1 and/or BRCA2 may be reduced or abolished in the cancer cells, for example by means of mutation or polymorphism in the encoding nucleic acid, or by means of amplification, mutation or polymorphism in a gene encoding a regulatory factor, for example the EMSY gene which encodes a BRCA2 regulatory factor (Hughes-Davies, et al., Cell, 115, 523-535) or by an epigenetic mechanism such as gene promoter methylation.

BRCA1 and BRCA2 are known tumour suppressors whose wild-type alleles are frequently lost in tumours of heterozygous carriers (Jasin M., Oncogene, 21(58), 8981-93 (2002); Tutt, et al., Trends Mol. Med., 8(12), 571-6, (2002)). The association of BRCA1 and/or BRCA2 mutations with breast cancer is well-characterised in the art (Radice, P. J., Exp. Clin. Cancer Res., 21(3 Suppl), 9-12 (2002)). Amplification of the EMSY gene, which encodes a BRCA2 binding factor, is also known to be associated with breast and ovarian cancer.

Carriers of mutations in BRCA1 and/or BRCA2 are also at elevated risk of cancer of the ovary, prostate and pancreas.

In some preferred embodiments, the individual is heterozygous for one or more variations, such as mutations and polymorphisms, in BRCA1 and/or BRCA2 or a regulator thereof. The detection of variation in BRCA1 and BRCA2 is well-known in the art and is described, for example in EP 699 754, EP 705 903, Neuhausen, S. L. and Ostrander, E. A., Genet. Test, 1, 75-83 (1992); Janatova M., et al., Neoplasma, 50(4), 246-50 (2003). Determination of amplification of the BRCA2 binding factor EMSY is described in Hughes-Davies, et al., Cell, 115, 523-535).

Mutations and polymorphisms associated with cancer may be detected at the nucleic acid level by detecting the presence of a variant nucleic acid sequence or at the protein level by detecting the presence of a variant (i.e. a mutant or allelic variant) polypeptide.

### DEFINITIONS

The term “aromatic ring” is used herein in the conventional sense to refer to a cyclic aromatic structure, that is, a cyclic structure having delocalised π-electron orbitals.

The aromatic ring fused to the main core, i.e. that formed by -A-B-, may bear further fused aromatic rings (resulting in, e.g. naphthyl or anthracenyl groups). The aromatic ring(s) may comprise solely carbon atoms, or may comprise carbon atoms and one or more heteroatoms, including but not limited to, nitrogen, oxygen, and sulfur atoms. The aromatic ring(s) preferably have five or six ring atoms.

The aromatic ring(s) may optionally be substituted. If a substituent itself comprises an aryl group, this aryl group is not considered to be a part of the aryl group to which it is attached. For example, the group biphenyl is considered herein to be a phenyl group (an aryl group comprising a single aromatic ring) substituted with a phenyl group. Similarly, the group benzylphenyl is considered to be a phenyl group (an aryl group comprising a single aromatic ring) substituted with a benzyl group.

In one group of preferred embodiments, the aromatic group comprises a single aromatic ring, which has five or six ring atoms, which ring atoms are selected from carbon, nitrogen, oxygen, and sulfur, and which ring is optionally substituted. Examples of these groups include, but are not limited to, benzene, pyrazine, pyrrole, thiazole, isoxazole, and oxazole. 2-Pyrone can also be considered to be an aromatic ring, but is less preferred.

If the aromatic ring has six atoms, then preferably at least four, or even five or all, of the ring atoms are carbon. The other ring atoms are selected from nitrogen, oxygen and sulphur, with nitrogen and oxygen being preferred. Suitable groups include a ring with: no hetero atoms (benzene); one nitrogen ring atom (pyridine); two nitrogen ring atoms (pyrazine, pyrimidine and pyridazine); one oxygen ring atom (pyrone); and one oxygen and one nitrogen ring atom (oxazine).

If the aromatic ring has five ring atoms, then preferably at least three of the ring atoms are carbon. The remaining ring atoms are selected from nitrogen, oxygen and sulphur. Suitable rings include a ring with: one nitrogen ring atom (pyrrole); two nitrogen ring atoms (imidazole, pyrazole); one oxygen ring atom (furan); one sulphur ring atom (thiophene); one nitrogen and one sulphur ring atom (isothiazole, thiazole); and one nitrogen and one oxygen ring atom (isoxazole or oxazole).

The aromatic ring may bear one or more substituent groups at any available ring position. These substituents are selected from halo, nitro, hydroxy, ether, thiol, thioether, amino, C₁₋₇ alkyl, C₃₋₂₀ heterocyclyl and C₅₋₂₀ aryl. The aromatic ring may also bear one or more substituent groups which together form a ring. In particular these may be of formula —(CH₂)ₘ— or —O—(CH₂)ₚ—O—, where m is 2, 3, 4 or 5 and p is 1, 2 or 3.

The term “fused cyclohexene ring” refers to a compound where the group -A-B- represents —(CH₂)₄—. If the group is substituted, one or more of the hydrogen atoms is replaced by an alternative group. The fused cyclohexene ring may bear one or more substituent groups at any available ring position. These substituents are selected from halo, nitro, hydroxy, ether, thiol, thioether, amino, C₁₋₇ alkyl, C₃₋₂₀ heterocyclyl and C₅₋₂₀ aryl. The fused cyclohexene ring may also bear one or more substituent groups which together form a ring. In particular these may be of formula —(CH₂)ₘ— or —O—(CH₂)ₚ—O—, where m is 2, 3, 4 or 5 and p is 1, 2 or 3.

Nitrogen-containing C₅₋₇ heterocyclylic ring: The term “nitrogen-containing C₅₋₇ heterocyclylic ring” as used herein, pertains to a C₅₋₇ heterocyclylic ring, as defined below with relation to heterocyclyl, having at least one nitrogen ring atom.

Alkyl: The term “alkyl” as used herein, pertains to a monovalent moiety obtained by removing a hydrogen atom from a carbon atom of a hydrocarbon compound having from 1 to 20 carbon atoms (unless otherwise specified), which may be aliphatic or alicyclic, and which may be saturated or unsaturated (e.g. partially unsaturated, fully unsaturated). Thus, the term “alkyl” includes the sub-classes alkenyl, alkynyl, cycloalkyl, cycloalkyenyl, cylcoalkynyl, etc., discussed below.

In the context of alkyl groups, the prefixes (e.g. C₁₋₄, C₁₋₇, C₁₋₂₀, C₂₋₇, C₃₋₇, etc.) denote the number of carbon atoms, or range of number of carbon atoms. For example, the term “C₁₋₄ alkyl”, as used herein, pertains to an alkyl group having from 1 to 4 carbon atoms. Examples of groups of alkyl groups include C₁₋₄ alkyl (“lower alkyl”), C₁₋₇ alkyl, and C₁₋₂₀ alkyl. Note that the first prefix may vary according to other limitations; for example, for unsaturated alkyl groups, the first prefix must be at least 2; for cyclic alkyl groups, the first prefix must be at least 3; etc.

Examples of (unsubstituted) saturated alkyl groups include, but are not limited to, methyl (C₁), ethyl (C₂), propyl (C₃), butyl (C₄), pentyl (C₅), hexyl (C₆), heptyl (C₇), octyl (C₈), nonyl (C₉), decyl (C₁₀), undecyl (C₁₁), dodecyl (C₁₂), tridecyl (C₁₃), tetradecyl (C₁₄), pentadecyl (C₁₅), and eicodecyl (C₂₀).

Examples of (unsubstituted) saturated linear alkyl groups include, but are not limited to, methyl (C₁), ethyl (C₂), n-propyl (C₃), n-butyl (C₄), n-pentyl (amyl) (C₅), n-hexyl (C₆), and n-heptyl (C₇).

Examples of (unsubstituted) saturated branched alkyl groups include, but are not limited to, iso-propyl (C₃), iso-butyl (C₄), sec-butyl (C₄), tert-butyl (C₄), iso-pentyl (C₅), and neo-pentyl (C₅).

Alkenyl: The term “alkenyl”, as used herein, pertains to an alkyl group having one or more carbon-carbon double bonds. Examples of alkenyl groups include C₂₋₄ alkenyl, C₂₋₇ alkenyl, C₂₋₂₀ alkenyl.

Examples of (unsubstituted) unsaturated alkenyl groups include, but are not limited to, ethenyl (vinyl, —CH═CH₂), 1-propenyl (—CH═CH—CH₃), 2-propenyl (allyl, —CH—CH═CH₂), isopropenyl (1-methylvinyl, —C(CH₃)═CH₂), butenyl (C₄), pentenyl (C₅), and hexenyl (C₆).

Alkynyl: The term “alkynyl”, as used herein, pertains to an alkyl group having one or more carbon-carbon triple bonds. Examples of alkynyl groups include C₂₋₄ alkynyl, C₂₋₇ alkynyl, C₂₋₂₀ alkynyl.

Examples of (unsubstituted) unsaturated alkynyl groups include, but are not limited to, ethynyl (ethinyl, —C≡CH) and 2-propynyl (propargyl, —CH₂—C≡CH).

Cycloalkyl: The term “cycloalkyl”, as used herein, pertains to an alkyl group which is also a cyclyl group; that is, a monovalent moiety obtained by removing a hydrogen atom from an alicyclic ring atom of a carbocyclic ring of a carbocyclic compound, which carbocyclic ring may be saturated or unsaturated (e.g. partially unsaturated, fully unsaturated), which moiety has from 3 to 20 carbon atoms (unless otherwise specified), including from 3 to 20 ring atoms. Thus, the term “cycloalkyl” includes the sub-classes cycloalkenyl and cycloalkynyl. Preferably, each ring has from 3 to 7 ring atoms. Examples of groups of cycloalkyl groups include C₃₋₂₀ cycloalkyl, C₃₋₁₅ cycloalkyl, C₃₋₁₀ cycloalkyl, C₃₋₇ cycloalkyl.

### Examples of cycloalkyl groups include, but are not limited to, those derived from

saturated monocyclic hydrocarbon compounds:
   cyclopropane (C₃), cyclobutane (C₄), cyclopentane (C₅), cyclohexane (C₆), cycloheptane (C₇), methylcyclopropane (C₄), dimethylcyclopropane (C₅), methylcyclobutane (C₅), dimethylcyclobutane (C₆), methylcyclopentane (C₆), dimethylcyclopentane (C₇), methylcyclohexane (C₇), dimethylcyclohexane (C₈), menthane (C₁₀);
unsaturated monocyclic hydrocarbon compounds:
   cyclopropene (C₃), cyclobutene (C₄), cyclopentene (C₅), cyclohexene (C₆), methylcyclopropene (C₄), dimethylcyclopropene (C₅), methylcyclobutene (C₅), dimethylcyclobutene (C₆), methylcyclopentene (C₆), dimethylcyclopentene (C₇), methylcyclohexene (C₇), dimethylcyclohexene (C₈);
saturated polycyclic hydrocarbon compounds:
   thujane (C₁₀), carane (C₁₀), pinane (C₁₀), bornane (C₁₀), norcarane (C₇), norpinane (C₇), norbornane (C₇), adamantane (C₁₀), decalin (decahydronaphthalene) (C₁₀);
unsaturated polycyclic hydrocarbon compounds:
   camphene (C₁₀), limonene (C₁₀), pinene (C₁₀);
polycyclic hydrocarbon compounds having an aromatic ring:
   indene (C₉), indane (e.g., 2,3-dihydro-1H-indene) (C₉), tetraline (1,2,3,4-tetrahydronaphthalene) (C₁₀), acenaphthene (C₁₂), fluorene (C₁₃), phenalene (C₁₃), acephenanthrene (C₁₅), aceanthrene (C₁₆), cholanthrene (C₂₀).

Heterocyclyl: The term “heterocyclyl”, as used herein, pertains to a monovalent moiety obtained by removing a hydrogen atom from a ring atom of a heterocyclic compound, which moiety has from 3 to 20 ring atoms (unless otherwise specified), of which from 1 to 10 are ring heteroatoms. Preferably, each ring has from 3 to 7 ring atoms, of which from 1 to 4 are ring heteroatoms.

In this context, the prefixes (e.g. C₃₋₂₀, C₃₋₇, C₅₋₆, etc.) denote the number of ring atoms, or range of number of ring atoms, whether carbon atoms or heteroatoms. For example, the term “C₅₋₆heterocyclyl”, as used herein, pertains to a heterocyclyl group having 5 or 6 ring atoms. Examples of groups of heterocyclyl groups include C₃₋₂₀ heterocyclyl, C₅₋₂₀ heterocyclyl, C₃₋₁₅ heterocyclyl, C₅₋₁₅ heterocyclyl, C₃₋₁₂ heterocyclyl, C₅₋₁₂ heterocyclyl, C₃₋₁₀ heterocyclyl, C₅₋₁₀ heterocyclyl, C₃₋₇ heterocyclyl, C₅₋₇ heterocyclyl, and C₅₋₆ heterocyclyl.

Examples of monocyclic heterocyclyl groups include, but are not limited to, those derived from:

N₁: aziridine (C₃), azetidine (C₄), pyrrolidine (tetrahydropyrrole) (C₅), pyrroline (e.g., 3-pyrroline, 2,5-dihydropyrrole) (C₅), 2H-pyrrole or 3H-pyrrole (isopyrrole, isoazole) (C₅), piperidine (C₆), dihydropyridine (C₆), tetrahydropyridine (C₆), azepine (C₇);
O₁: oxirane (C₃), oxetane (C₄), oxolane (tetrahydrofuran) (C₅), oxole (dihydrofuran) (C₅), oxane (tetrahydropyran) (C₆), dihydropyran (C₆), pyran (C₆), oxepin (C₇);
S₁: thiirane (C₃), thietane (C₄), thiolane (tetrahydrothiophene) (C₅), thiane (tetrahydrothiopyran) (C₆), thiepane (C₇);
O₂: dioxolane (C₅), dioxane (C₆), and dioxepane (C₇);
O₃: trioxane (C₆);
N₂: imidazolidine (C₅), pyrazolidine (diazolidine) (C₅), imidazoline (C₅), pyrazoline (dihydropyrazole) (C₅), piperazine (C₆);
N₁O₁: tetrahydrooxazole (C₅), dihydrooxazole (C₅), tetrahydroisoxazole (C₅), dihydroisoxazole (C₅), morpholine (C₆), tetrahydrooxazine (C₆), dihydrooxazine (C₆), oxazine (C₆);
N₁S₁: thiazoline (C₅), thiazolidine (C₅), thiomorpholine (C₆);
N₂O₁: oxadiazine (C₆);
O₁S₁: oxathiole (C₅) and oxathiane (thioxane) (C₆); and,
N₁O₁S₁: oxathiazine (C₆).

Examples of substituted (non-aromatic) monocyclic heterocyclyl groups include those derived from saccharides, in cyclic form, for example, furanoses (C₅), such as arabinofuranose, lyxofuranose, ribofuranose, and xylofuranse, and pyranoses (C₆), such as allopyranose, altropyranose, glucopyranose, mannopyranose, gulopyranose, idopyranose, galactopyranose, and talopyranose.

C₅₋₂₀ aryl: The term “C₅₋₂₀aryl” as used herein, pertains to a monovalent moiety obtained by removing a hydrogen atom from an aromatic ring atom of a C₅₋₂₀ aromatic compound, said compound having one ring, or two or more rings (e.g., fused), and having from 5 to 20 ring atoms, and wherein at least one of said ring(s) is an aromatic ring. Preferably, each ring has from 5 to 7 ring atoms.

The ring atoms may be all carbon atoms, as in “carboaryl groups” in which case the group may conveniently be referred to as a “C₅₋₂₀ carboaryl” group.

Examples of C₅₋₂₀ aryl groups which do not have ring heteroatoms (i.e. C₅₋₂₀ carboaryl groups) include, but are not limited to, those derived from benzene (i.e. phenyl) (C₆), naphthalene (C₁₀), anthracene (C₁₄), phenanthrene (C₁₄), and pyrene (C₁₆).

Alternatively, the ring atoms may include one or more heteroatoms, including but not limited to oxygen, nitrogen, and sulfur, as in “heteroaryl groups”. In this case, the group may conveniently be referred to as a “C₅₋₂₀ heteroaryl” group, wherein “C₅₋₂₀” denotes ring atoms, whether carbon atoms or heteroatoms. Preferably, each ring has from 5 to 7 ring atoms, of which from 0 to 4 are ring heteroatoms.

Examples of C₅₋₂₀ heteroaryl groups include, but are not limited to, C₅ heteroaryl groups derived from furan (oxole), thiophene (thiole), pyrrole (azole), imidazole (1,3-diazole), pyrazole (1,2-diazole), triazole, oxazole, isoxazole, thiazole, isothiazole, oxadiazole, tetrazole and oxatriazole; and C₆ heteroaryl groups derived from isoxazine, pyridine (azine), pyridazine (1,2-diazine), pyrimidine (1,3-diazine; e.g., cytosine, thymine, uracil), pyrazine (1,4-diazine) and triazine.

The heteroaryl group may be bonded via a carbon or hetero ring atom, when present as an optional substituent, i.e. not when it is R^{D}.

Examples of C₅₋₂₀ heteroaryl groups which comprise fused rings, include, but are not limited to, C₉ heteroaryl groups derived from benzofuran, isobenzofuran, benzothiophene, indole, isoindole; C₁₀ heteroaryl groups derived from quinoline, isoquinoline, benzodiazine, pyridopyridine; C₁₁ heteroaryl groups derived from dibenzofuran and dibenzothiophene; C₁₋₄ heteroaryl groups derived from acridine, thianthrene, phenoxathiine and xanthene.

The above alkyl, heterocyclyl, and aryl groups, whether alone or part of another substituent, may themselves optionally be substituted with one or more groups selected from themselves and the additional substituents listed below.

Halo: —F, —Cl, —Br, and —I.

Hydroxy: —OH.

Ether: —OR, wherein R is an ether substituent, for example, a C₁₋₇ alkyl group (also referred to as a C₁₋₇ alkoxy group), a C₃₋₂₀ heterocyclyl group (also referred to as a C₃₋₂₀ heterocyclyloxy group), or a C₅₋₂₀ aryl group (also referred to as a C₅₋₂₀ aryloxy group), preferably a C₁₋₇ alkyl group.

Nitro: —NO₂.

Cyano (nitrile, carbonitrile): —CN.

Acyl (keto): —C(═O)R, wherein R is an acyl substituent, for example, H, a C₁₋₇ alkyl group (also referred to as C₁₋₇ alkylacyl or C₁₋₇ alkanoyl), a C₃₋₂₀ heterocyclyl group (also referred to as C₃₋₂₀ heterocyclylacyl), or a C₅₋₂₀ aryl group (also referred to as C₅₋₂₀ arylacyl), preferably a C₁₋₇ alkyl group. Examples of acyl groups include, but are not limited to, —C(═O)CH₃ (acetyl), —C(═O)CH₂CH₃ (propionyl), —C(═O)C(CH₃)₃ (butyryl), and —C(═O)Ph (benzoyl, phenone).

Carboxy (carboxylic acid): —COOH.

Ester (carboxylate, carboxylic acid ester, oxycarbonyl): —C(═O)OR, wherein R is an ester substituent, for example, a C₁₋₇ alkyl group, a C₃₋₂₀ heterocyclyl group, or a C₅₋₂₀ aryl group, preferably a C₁₋₇ alkyl group. Examples of ester groups include, but are not limited to, —C(═O)OCH₃, —C(═O)OCH₂CH₃, —C(═O)OC(CH₃)₃, and —C(═O)OPh.

Amido (carbamoyl, carbamyl, aminocarbonyl, carboxamide): —C(═O)NR¹R², wherein R¹ and R² are independently amino substituents, as defined for amino groups. Examples of amido groups include, but are not limited to, —C(═O)NH₂, —C(═O)NHCH₃, —C(═O)N(CH₃)₂, —C(═O)NHCH₂CH₃, and —C(═O)N(CH₂CH₃)₂, as well as amido groups in which R¹ and R², together with the nitrogen atom to which they are attached, form a heterocyclic structure as in, for example, piperidinocarbonyl, morpholinocarbonyl, thiomorpholinocarbonyl, and piperazinylcarbonyl.

Amino: —NR¹R², wherein R¹ and R² are independently amino substituents, for example, hydrogen, a C₁₋₇ alkyl group (also referred to as C₁₋₇ alkylamino or di-C₁₋₇ alkylamino), a C₃₋₂₀ heterocyclyl group, or a C₅₋₂₀ aryl group, preferably H or a C₁₋₇ alkyl group, or, in the case of a “cyclic” amino group, R¹ and R², taken together with the nitrogen atom to which they are attached, form a heterocyclic ring having from 4 to 8 ring atoms. Examples of amino groups include, but are not limited to, —NH₂, —NHCH₃, —NHCH(CH₃)₂, —N(CH₃)₂, —N(CH₂CH₃)₂, and —NHPh. Examples of cyclic amino groups include, but are not limited to, aziridinyl, azetidinyl, pyrrolidinyl, piperidino, piperazinyl, perhydrodiazepinyl, morpholino, and thiomorpholino. In particular, the cyclic amino groups may be substituted on their ring by any of the substituents defined here, for example carboxy, carboxylate and amido.

Acylamido (acylamino): —NR¹C(═O)R², wherein R¹ is an amide substituent, for example, hydrogen, a C₁₋₇ alkyl group, a C₃₋₂₀ heterocyclyl group, or a C₅₋₂₀ aryl group, preferably H or a C₁₋₇ alkyl group, most preferably H, and R² is an acyl substituent, for example, a C₁₋₇ alkyl group, a C₃₋₂₀ heterocyclyl group, or a C₅₋₂₀ aryl group, preferably a C₁₋₇ alkyl group. Examples of acylamide groups include, but are not limited to, —NHC(═O)CH₃, —NHC(═O)CH₂CH₃, and —NHC(═O)Ph. R¹ and R² may together form a cyclic structure, as in, for example, succinimidyl, maleimidyl, and phthalimidyl:

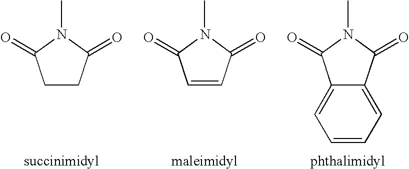

Ureido: —N(R¹)CONR²R³ wherein R² and R³ are independently amino substituents, as defined for amino groups, and R¹ is a ureido substituent, for example, hydrogen, a C₁₋₇alkyl group, a C₃₋₂₀heterocyclyl group, or a C₅₋₂₀aryl group, preferably hydrogen or a C₁₋₇alkyl group. Examples of ureido groups include, but are not limited to, —NHCONH₂, —NHCONHMe, —NHCONHEt, —NHCONMe₂, —NHCONEt₂, —NMeCONH₂, —NMeCONHMe, —NMeCONHEt, —NMeCONMe₂, —NMeCONEt₂ and —NHCONHPh.

Acyloxy (reverse ester): —OC(═O)R, wherein R is an acyloxy substituent, for example, a C₁₋₇ alkyl group, a C₃₋₂₀ heterocyclyl group, or a C₅₋₂₀ aryl group, preferably a C₁₋₇ alkyl group. Examples of acyloxy groups include, but are not limited to, —OC(═O)CH₃ (acetoxy), —OC(═O)CH₂CH₃, —OC(═O)C(CH₃)₃, —OC(═O)Ph, —OC(═O)C₆H₄F, and —OC(═O)CH₂Ph.

Thiol: —SH.

Thioether (sulfide): —SR, wherein R is a thioether substituent, for example, a C₁₋₇ alkyl group (also referred to as a C₁₋₇ alkylthio group), a C₃₋₂₀ heterocyclyl group, or a C₅₋₂₀ aryl group, preferably a C₁₋₇ alkyl group. Examples of C₁₋₇ alkylthio groups include, but are not limited to, —SCH₃ and —SCH₂CH₃.

Sulfoxide (sulfinyl): —S(═O)R, wherein R is a sulfoxide substituent, for example, a C₁₋₇ alkyl group, a C₃₋₂₀ heterocyclyl group, or a C₅₋₂₀ aryl group, preferably a C₁₋₇ alkyl group. Examples of sulfoxide groups include, but are not limited to, —S(═O)CH₃ and —S(═O)CH₂CH₃.

Sulfonyl (sulfone): —S(═O)₂R, wherein R is a sulfone substituent, for example, a C₁₋₇ alkyl group, a C₃₋₂₀ heterocyclyl group, or a C₅₋₂₀ aryl group, preferably a C₁₋₇ alkyl group. Examples of sulfone groups include, but are not limited to, —S(═O)₂CH₃ (methanesulfonyl, mesyl), —S(═O)₂CF₃, —S(═O)₂CH₂CH₃, and 4-methylphenylsulfonyl (tosyl).

Thioamido (thiocarbamyl): —C(═S)NR¹R², wherein R¹ and R² are independently amino substituents, as defined for amino groups. Examples of amido groups include, but are not limited to, —C(═S)NH₂, —C(═S)NHCH₃, —C(═S)N(CH₃)₂, and —C(═S)NHCH₂CH₃.

Sulfonamino: —NR¹S(═O)₂R, wherein R¹ is an amino substituent, as defined for amino groups, and R is a sulfonamino substituent, for example, a C₁₋₇alkyl group, a C₃₋₂₀heterocyclyl group, or a C₅₋₂₀aryl group, preferably a C₁₋₇alkyl group. Examples of sulfonamino groups include, but are not limited to, —NHS(═O)₂CH₃, —NHS(═O)₂Ph and —N(CH₃)S(═O)₂C₆H₅.

Dioxyalkylene: —O(CH₂)ₙ—, wherein n is an integer from 1 to 3.

Oxyalkylene: —O(CH₂)ₙ—, wherein n is an integer from 2 to 4.

As mentioned above, the groups that form the above listed substituent groups, e.g. C₁₋₇ alkyl, C₃₋₂₀ heterocyclyl and C₅₋₂₀ aryl, may themselves be substituted. Thus, the above definitions cover substituent groups which are substituted.

### FURTHER EMBODIMENTS

The following embodiments can relate to each aspect of the present invention, where applicable.

In the present invention, the fused aromatic ring(s) represented by -A-B- preferably consist of solely carbon ring atoms, and thus may be benzene, naphthalene, and is more preferably benzene. As described above, these rings may be substituted, but in some embodiments are preferably unsubstituted.

If the fused aromatic ring represented by -A-B- bears one or more substituent groups, these are preferably attached to the atoms which themselves is attached to the central ring β- to the carbon atom in the central ring. Thus, if the fused aromatic ring is a benzene ring, the preferred place of substitution is shown in the formula below by *:

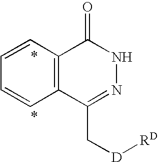

These substituent are preferably halo groups, and more preferably F.

If the fused cyclohexene ring represented by -A-B- bears a single substituent, the compound can be of the formula:

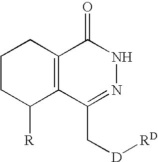

where R represents the optional substituent.

If the fused cyclohexene ring represented by -A-B- bears a substituent, the substituent may be selected from halo, hydroxy and amino (e.g. NH₂).

D may be selected from:

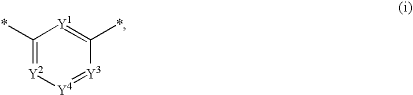

where Y¹ is selected from CH and N, Y² is selected from CH and N, Y³ is selected from CH, CF and N, Y⁴ is selected from CH and N but where only one of Y¹, Y², Y³ and Y⁴ may be N; and

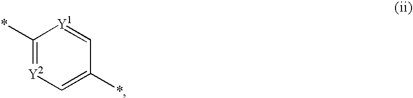

where Y¹ is selected from CH and N and Y² is selected from CH and N, but where only one of Y¹ and Y² may be N.

In particular, D may be selected from:

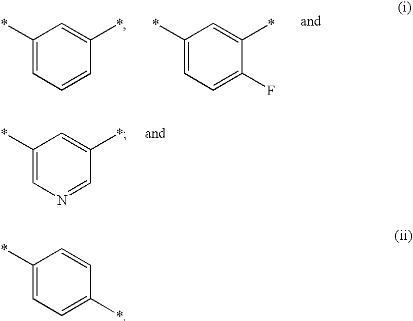

D may also be:

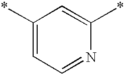

D may also be selected from:

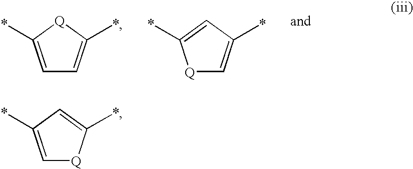

where Q is S; and

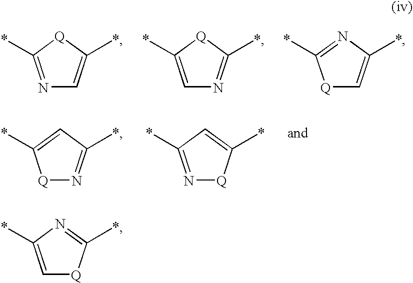

where Q is S.

In particular, D may also be selected from:

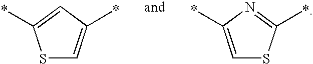

In some embodiments, R^{D} may contain 0, 1 or 2 ring heteroatoms.

R^{D} may be selected from a C₅₋₂₀ carboaryl group, such as phenyl and naphthyl. The napthyl group may be a napht-1-yl or naphth-2-yl group.

R^{D} may also be selected from a C₅ heteroaryl group, such as furanyl (furan-2-yl, furan-3-yl), thiophenyl (thiophen-2-yl, thiophen-3-yl), pyrrolyl (pyrrol-2-yl, pyrrol-3-yl).

R^{D} may also be selected from a C₆ heteroaryl group, such as pyridyl (pyrid-2-yl, pyrid-3-yl, pyrid-4-yl), pyrazinyl (pyrazin-2-yl) and pyrimidinyl (pyrimidin-4-yl).

R^{D} may also be selected from a C₉ heteroaryl group, such as benzothiophenyl (benzothiophen-2-yl, benzothiophen-3-yl) and indolyl (indol-5-yl).

R^{D} may also be selected from a C₁₀ heteroaryl group, such as quinolinyl (quinolin-5-yl, quinolin-8-yl) and isoquinolinyl (isoquinolin-5-yl).

R^{D} may also be selected from a C₁₁ heteroaryl group, such as dibenzofuranyl (dibenzofuran-4-yl) and dibenzothiophenyl (dibenzothiophen-4-yl).

R^{D} may also be selected from a C₁₋₄ heteroaryl group, such as thianthrenyl (thianthren-1-yl) and phenoxathiinyl (phenoxathiin-4-yl).

The optional substituents for R^{D} may be selected from C₁₋₂₀ alkyl, C₃₋₂₀ heterocyclyl, C₅₋₂₀ aryl, ester, ether, cyano, acyl, acylamido, halo, nitro, dioxyalkylene, oxyalkylene, amido, sulfonyl, thioether, amino, sulfonamino, ureido, carboxy and hydroxyl.

When R^{D} is phenyl, it may be unsubstituted or substituted by one or two groups selected from C₁₋₂₀ alkyl, C₅₋₂₀ aryl, ester, ether, cyano, acyl, acylamido, halo, nitro, dioxyalkylene, oxyalkylene, amido, sulfonyl, thioether, amino, sulfonamino, ureido, carboxy and hydroxyl.

When R^{D} is napthyl, it may be unsubstituted or substituted by one or two groups selected from C₁₋₂₀ alkyl (methyl), ether (methoxy) and acyl (—COH).

When R^{D} is pyridyl, it may be unsubstituted or substituted with one or two halo (Cl) groups.

When R^{D} is pyrrolyl, it may be unsubstituted or substituted with a group selected from C₁₋₂₀ alkyl (methyl) or ester (t-butyloxycarbonyl). These groups may be N-substituents.

When R^{D} is thiophenyl, it may be unsubstituted or substituted with one or two groups selected from halo (Cl), acyl (methylcarbonyl) or phenyl, where the phenyl group may itself be substituted with a group selected from acyl (methylcarbonyl), C₁₋₇ alkyl (hydroxymethyl, aminomethyl) and amido (e.g., with a hydrogen and furanylmethyl amino substituents).

When R^{D} is furanyl, it may be substituted with one or two acyl (methylcarbonyl) groups.

When R^{D} is benzothiophenyl, pyrazinyl, pyrimidinyl, thianthrenyl, dibenzofuranyl, dibenothiophenyl, phenoxathiine, quinolinyl, isoquinolinyl, and indolyl, these may be unsubstituted.

When R^{D} is phenyl, and R^{D} is meta to the attachment point of D to the remainder of the molecule (i.e. D is selected from (i)), it may have a single substituent, selected from C₁₋₂₀ alkyl, C₅₋₂₀ aryl, ester, ether, cyano, acyl, acylamido, halo, nitro, dioxyalkylene, oxyalkylene, amido, sulfonyl, thioether, amino, sulfonamino and ureido.

If the substituent is nitro, it may be in the para position.

If the substituent is cyano, it may be in the meta or para positions.

If the substituent is halo, it may be chloro, which may be in the ortho, meta or para positions, or fluoro, which may be in the para position.

If the substituent is ester, the ester substituent may be methyl (in which case, the ester may be in the ortho or para positions), ethyl (in which case, the ester may be in the meta position) or benzyl (in which case, the ester may be in the para position).

If the substituent is ether, the ether substituent may be methyl (in which case, the ether may be in the ortho, meta or para positions), trifluoromethyl (in which case, the ether may be in the ortho, meta or para positions), benzyl (in which case, the ether may be in the ortho, meta or para positions) and phenyl (in which case, the ether may be in the ortho or para positions).

If the substituent is acyl, the acyl substituent may be hydrogen (in which case, the acyl may be in the ortho or para positions) or methyl (in which case, the ether may be in the ortho, meta or para positions).

If the substituent is C₅₋₂₀ aryl, then it may be phenyl, which may be in the ortho or para positions.

If the substituent is C₁₋₂₀ alkyl, then it may be substituted or unsubstituted, and may be a C₁₋₂ alkyl group. The optional substituents may include hydroxy, alkoxy, halo, ester and acyl. In particular, the group may be hydroxymethyl (in which case, the group may be in the ortho or meta positions), t-butoxymethyl (in which case, the group may be in the meta position), methoxymethyl (in which case, the group may be in the para position), methyl (in which case, the group may be in the ortho or para positions), trifluoromethyl (in which case, the group may be in the meta or para positions), methoxycarbonlyethenyl (in which case, the group may be in the ortho, meta or para positions) and ethylcarbonylethenyl (in which case, the group may be in the meta position).

If the substituent is sulfonyl, then the sulfone substituent may be methyl (in which case, the sulfonyl may be in the meta position).

If the substituent is thioether, then the thioether substituent may be methyl (in which case, the thioether may be in the meta or para positions).

If the substituent is amino, then both amino substituents may be hydrogen (in which case, the amino may be in the meta or para positions) or methyl (in which case, the amino may be in the meta position).

If the substituent is dioxyalkylene, this may link the meta and para positions, and may have n=2 or 3.

If the substituent is oxyalkylene, this may link the meta and ortho positions, and may have n=2.

If the substituent is acylamido, the amide substituent is usually hydrogen. The acyl substituent may be hydrogen (in which case, the acylamido may be in the ortho position), or it may be selected from C₁₋₂₀ alkyl, C₃₋₂₀ heterocyclyl or C₅₋₂₀ aryl (in which case, the acylamido may be in the meta or para positions). If the C₁₋₂₀ alkyl is methyl, then the acylamido may be in the ortho, meta or para positions. The C₁₋₂₀ alkyl acyl substituent may be selected from C₁₋₇ alkyl groups, which may be optionally substituted with amino, and C₅₋₂₀ aryl groups, such that the groups are, e.g., methyl, dimethylaminomethyl, pyridylmethyl, indolylmethyl, phenylethyl, piperidinylethyl, phenoxy-1-ethyl, cyclopropyl, phenylpropyl, methylphenylethyl, thiophenylmethyl, 2-methylhexyl. Further examples include phenylmethyl. Furthermore, the C₁₋₇ alkyl acyl substituent may be substituted with a C₃₋₇ heterocyclyl group, such that the groups are, e.g. tetrahydrofuranylmethyl, N-piperidinylethyl. The C₅₋₂₀ aryl acyl substituent may be selected from C₅₋₆ aryl groups, such as furanyl, methylfuranyl, phenyl, fluorophenyl, fluoropyridyl, pyrazinyl, N-methylpyrazolyl, methyl N-methylpyrazolyl, tetrazolyl, thiophenyl, isoxazolyl, pyridyl, pyrimidinyl, cyanophenyl and dimethylaminophenyl. The C₃₋₂₀ heterocylyl acyl substituent may be selected from C₆₋₁₂ heterocylyl groups, such as N-methylpiperidinyl and chomanyl.

If the substituent is amido, it may be, for example, the two amino substituents, together with the nitrogen to which they are bound, may form 4-methylpiperidine (in which case, the amido may be in the meta position), thiomorpholino (in which case, the amido may be in the meta position) or pyrrolidinyl (in which case, the amido may be in the meta position). Other amido substituents, may have the two amino substituents and the nitrogen atom to which they are bound forming a piperzine or homopiperazine group, which may bear a N-substituent, such as C₁₋₄ alkyl (methyl, ethyl, hydroxyethyl, methoxyethyl) or acyl (methylcarbonyl), in which case the amido may be in the meta or para positions. Other amido substituents may have one amino substituent as hydrogen or methyl and the other as aminoethyl (e.g. dimethylaminoethyl, pyrrolidinylethyl), in which case the amido may be in the meta or para positions.

If the substituent is sulfonamino, the amino substituent is usually hydrogen. The sulfonamino substituent may be selected from C₁₋₇ alkyl (ethyl, butyl, phenylmethyl) and C₅₋₂₀ aryl (thiophenyl, chlorothiophenyl, phenyl, chlorophenyl, fluorophenyl, cyanophenyl, oxazolyl, dimethyloxazolyl, dimethylthiazolyl, dimethylpyrazolyl, benzooxadiazolyl, methoxyphenyl). The sulfonamino group may be in the meta or para positions. The sulfonamino substituent may itself be substituted. A particular class of sulfonamino substituents of use in the present invention are methyl or ethyl groups terminally substituted with a C₃₋₇ cycloalkyl (e.g. cyclopropyl, cyclopentyl, cyclohexyl), C₃₋₇ heterocyclyl group (tetrahydrofuranyl, tetrahydropyranyl, piperidinyl) and C₅₋₇ aryl (e.g. phenyl).

If the substituent is ureido, the ureido substituent is usually hydrogen. One amino substituent may be hydrogen with the other amino substituent being selected from C₁₋₇ alkyl (cyclohexyl, phenylcyclopropyl) and C₅₋₆ aryl (phenyl, fluorophenyl, cyanophenyl, dioxyethylenephenyl). The ureido may be in the meta or para positions.

When R^{D} is phenyl, and R^{D} is meta to the attachment point of D to the remainder of the molecule (i.e. D is selected from (i)), it may have two substituents selected from C₁₋₄ alkyl (methyl), halo, C₁₋₄ alkoxy (methoxy) and cyano. The substituent patterns may include: meta-fluoro, para-methoxy; meta-fluoro, para-cyano; ortho-methyl, meta-methyl; meta-methyl, meta-methyl.

When R^{D} is phenyl, and R^{D} is para to the attachment point of D to the remainder of the molecule (i.e. D is selected from (ii)), it may have a single substituent selected from amino, hydroxy, carboxy, and amido. These substituents may be in the meta position. The amino substituent on the amido group may form with the nitrogen atom to which they are attached a C₅₋₇ heterocyclyl group (4-methylpiperidine, thiomorpholine, pyrrolidine, piperidine) or one may be hydrogen and the other C₅₋₇ aryl (furanyl).

### Further Preferred Compounds

The compounds of the examples are preferred embodiments of the invention, as are the general formulae shown in the examples.

In particular, compounds where D is:

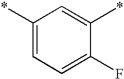

and R^{D} is phenyl with a para-sulfonamino group or furanyl with an aminomethyl substituent are preferred.

### Includes Other Forms

Included in the above are the well known ionic, salt, solvate, and protected forms of these substituents. For example, a reference to carboxylic acid (—COOH) also includes the anionic (carboxylate) form (—COO⁻), a salt or solvate thereof, as well as conventional protected forms. Similarly, a reference to an amino group includes the protonated form (—N⁺HR¹R²), a salt or solvate of the amino group, for example, a hydrochloride salt, as well as conventional protected forms of an amino group. Similarly, a reference to a hydroxyl group also includes the anionic form (—O⁻), a salt or solvate thereof, as well as conventional protected forms of a hydroxyl group.

### Isomers, Salts, Solvates, Protected Forms, and Prodrugs

Certain compounds may exist in one or more particular geometric, optical, enantiomeric, diasterioisomeric, epimeric, stereoisomeric, tautomeric, conformational, or anomeric forms, including but not limited to, cis- and trans-forms; E- and Z-forms; c-, t-, and r-forms; endo- and exo-forms; R-, S-, and meso-forms; D- and L-forms; d- and l-forms; (+) and (−) forms; keto-, enol-, and enolate-forms; syn- and anti-forms; synclinal- and anticlinal-forms; α- and β-forms; axial and equatorial forms; boat-, chair-, twist-, envelope-, and halfchair-forms; and combinations thereof, hereinafter collectively referred to as “isomers” (or “isomeric forms”).

If the compound is in crystalline form, it may exist in a number of different polymorphic forms.

Note that, except as discussed below for tautomeric forms, specifically excluded from the term “isomers”, as used herein, are structural (or constitutional) isomers (i.e. isomers which differ in the connections between atoms rather than merely by the position of atoms in space). For example, a reference to a methoxy group, —OCH₃, is not to be construed as a reference to its structural isomer, a hydroxymethyl group, —CH₂OH. Similarly, a reference to ortho-chlorophenyl is not to be construed as a reference to its structural isomer, meta-chlorophenyl. However, a reference to a class of structures may well include structurally isomeric forms falling within that class (e.g., C₁₋₇ alkyl includes n-propyl and iso-propyl; butyl includes n-, iso-, sec-, and tert-butyl; methoxyphenyl includes ortho-, meta-, and para-methoxyphenyl).

The above exclusion does not pertain to tautomeric forms, for example, keto-, enol-, and enolate-forms, as in, for example, the following tautomeric pairs: keto/enol, imine/enamine, amide/imino alcohol, amidine/amidine, nitroso/oxime, thioketone/enethiol, N-nitroso/hyroxyazo, and nitro/aci-nitro.

Particularly relevant to the present invention is the tautomeric pair illustrated below:

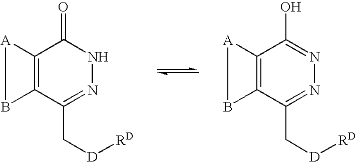

Note that specifically included in the term “isomer” are compounds with one or more isotopic substitutions. For example, H may be in any isotopic form, including ¹H, ²H (D), and ³H (T); C may be in any isotopic form, including ¹²C, ¹³C, and ¹⁴C; O may be in any isotopic form, including ¹⁶O and ¹³O; and the like.

Unless otherwise specified, a reference to a particular compound includes all such isomeric forms, including (wholly or partially) racemic and other mixtures thereof. Methods for the preparation (e.g. asymmetric synthesis) and separation (e.g. fractional crystallisation and chromatographic means) of such isomeric forms are either known in the art or are readily obtained by adapting the methods taught herein, or known methods, in a known manner.

Unless otherwise specified, a reference to a particular compound also includes ionic, salt, solvate, and protected forms of thereof, for example, as discussed below, as well as its different polymorphic forms.

It may be convenient or desirable to prepare, purify, and/or handle a corresponding salt of the active compound, for example, a pharmaceutically-acceptable salt. Examples of pharmaceutically acceptable salts are discussed in Berge, et al., “Pharmaceutically Acceptable Salts”, J. Pharm. Sci., 66, 1-19 (1977).

For example, if the compound is anionic, or has a functional group which may be anionic (e.g., —COOH may be —COO⁻), then a salt may be formed with a suitable cation. Examples of suitable inorganic cations include, but are not limited to, alkali metal ions such as Na⁺ and K⁺, alkaline earth cations such as Ca²⁺ and Mg²⁺, and other cations such as Al³⁺. Examples of suitable organic cations include, but are not limited to, ammonium ion (i.e., NH₄⁺) and substituted ammonium ions (e.g., NH₃R⁺, NH₂R₂⁺, NHR₃⁺, NR₄⁺). Examples of some suitable substituted ammonium ions are those derived from: ethylamine, diethylamine, dicyclohexylamine, triethylamine, butylamine, ethylenediamine, ethanolamine, diethanolamine, piperazine, benzylamine, phenylbenzylamine, choline, meglumine, and tromethamine, as well as amino acids, such as lysine and arginine. An example of a common quaternary ammonium ion is N(CH₃)₄⁺.

If the compound is cationic, or has a functional group which may be cationic (e.g., —NH₂ may be —NH₃⁺), then a salt may be formed with a suitable anion. Examples of suitable inorganic anions include, but are not limited to, those derived from the following inorganic acids: hydrochloric, hydrobromic, hydroiodic, sulfuric, sulfurous, nitric, nitrous, phosphoric, and phosphorous. Examples of suitable organic anions include, but are not limited to, those derived from the following organic acids: acetic, propionic, succinic, gycolic, stearic, palmitic, lactic, malic, pamoic, tartaric, citric, gluconic, ascorbic, maleic, hydroxymaleic, phenylacetic, glutamic, aspartic, benzoic, cinnamic, pyruvic, salicyclic, sulfanilic, 2-acetyoxybenzoic, fumaric, toluenesulfonic, methanesulfonic, ethanesulfonic, ethane disulfonic, oxalic, isethionic, valeric, and gluconic. Examples of suitable polymeric anions include, but are not limited to, those derived from the following polymeric acids: tannic acid, carboxymethyl cellulose.

It may be convenient or desirable to prepare, purify, and/or handle a corresponding solvate of the active compound. The term “solvate” is used herein in the conventional sense to refer to a complex of solute (e.g. active compound, salt of active compound) and solvent. If the solvent is water, the solvate may be conveniently referred to as a hydrate, for example, a mono-hydrate, a di-hydrate, a tri-hydrate, etc.

It may be convenient or desirable to prepare, purify, and/or handle the active compound in a chemically protected form. The term “chemically protected form,” as used herein, pertains to a compound in which one or more reactive functional groups are protected from undesirable chemical reactions, that is, are in the form of a protected or protecting group (also known as a masked or masking group or a blocked or blocking group). By protecting a reactive functional group, reactions involving other unprotected reactive functional groups can be performed, without affecting the protected group; the protecting group may be removed, usually in a subsequent step, without substantially affecting the remainder of the molecule. See, for example, “Protective Groups in Organic Synthesis” (T. Green and P. Wuts; 3rd Edition; John Wiley and Sons, 1999).

For example, a hydroxy group may be protected as an ether (—OR) or an ester (—OC(═O)R), for example, as: a t-butyl ether; a benzyl, benzhydryl (diphenylmethyl), or trityl (triphenylmethyl)ether; a trimethylsilyl or t-butyldimethylsilyl ether; or an acetyl ester (—OC(═O)CH₃, —OAc).

For example, an aldehyde or ketone group may be protected as an acetal or ketal, respectively, in which the carbonyl group (>C═O) is converted to a diether (>C(OR)₂), by reaction with, for example, a primary alcohol. The aldehyde or ketone group is readily regenerated by hydrolysis using a large excess of water in the presence of acid.

For example, an amine group may be protected, for example, as an amide or a urethane, for example, as: a methyl amide (—NHCO—CH₃); a benzyloxy amide (—NHCO—OCH₂C₆H₅, —NH-Cbz); as a t-butoxy amide (—NHCO—OC(CH₃)₃, —NH-Boc); a 2-biphenyl-2-propoxy amide (—NHCO—OC(CH₃)₂C₆H₄C₆H₅, —NH-Bpoc), as a 9-fluorenylmethoxy amide (—NH-Fmoc), as a 6-nitroveratryloxy amide (—NH-Nvoc), as a 2-trimethylsilylethyloxy amide (—NH-Teoc), as a 2,2,2-trichloroethyloxy amide (—NH-Troc), as an allyloxy amide (—NH-Alloc), as a 2(-phenylsulphonyl)ethyloxy amide (—NH-Psec); or, in suitable cases, as an N-oxide (>NO.).

For example, a carboxylic acid group may be protected as an ester for example, as: a C₁₋₇ alkyl ester (e.g. a methyl ester; a t-butyl ester); a C₁₋₇ haloalkyl ester (e.g. a C₁₋₇ trihaloalkyl ester); a triC₁₋₇ alkylsilyl-C₁₋₇ alkyl ester; or a C₅₋₂₀ aryl-C₁₋₇ alkyl ester (e.g. a benzyl ester; a nitrobenzyl ester); or as an amide, for example, as a methyl amide.

For example, a thiol group may be protected as a thioether (—SR), for example, as: a benzyl thioether; an acetamidomethyl ether (—S—CH₂NHC(═O)CH₃).

It may be convenient or desirable to prepare, purify, and/or handle the active compound in the form of a prodrug. The term “prodrug”, as used herein, pertains to a compound which, when metabolised (e.g. in vivo), yields the desired active compound. Typically, the prodrug is inactive, or less active than the active compound, but may provide advantageous handling, administration, or metabolic properties.

For example, some prodrugs are esters of the active compound (e.g. a physiologically acceptable metabolically labile ester). During metabolism, the ester group (—C(═O)OR) is cleaved to yield the active drug. Such esters may be formed by esterification, for example, of any of the carboxylic acid groups (—C(═O)OH) in the parent compound, with, where appropriate, prior protection of any other reactive groups present in the parent compound, followed by deprotection if required. Examples of such metabolically labile esters include, but are not limited to, those wherein R is C₁₋₂₀ alkyl (e.g. -Me, -Et); C₁₋₇ aminoalkyl (e.g. aminoethyl; 2-(N,N-diethylamino)ethyl; 2-(4-morpholino)ethyl); and acyloxy-C₁₋₇ alkyl (e.g. acyloxymethyl; acyloxyethyl; e.g. pivaloyloxymethyl; acetoxymethyl; 1-acetoxyethyl; 1-(1-methoxy-1-methyl)ethyl-carbonxyloxyethyl; 1-(benzoyloxy)ethyl; isopropoxy-carbonyloxymethyl; 1-isopropoxy-carbonyloxyethyl; cyclohexyl-carbonyloxymethyl; 1-cyclohexyl-carbonyloxyethyl; cyclohexyloxy-carbonyloxymethyl; 1-cyclohexyloxy-carbonyloxyethyl; (4-tetrahydropyranyloxy) carbonyloxymethyl; 1-(4-tetrahydropyranyloxy)carbonyloxyethyl; (4-tetrahydropyranyl)carbonyloxymethyl; and 1-(4-tetrahydropyranyl)carbonyloxyethyl).

Further suitable prodrug forms include phosphonate and glycolate salts. In particular, hydroxy groups (—OH), can be made into phosphonate prodrugs by reaction with chlorodibenzylphosphite, followed by hydrogenation, to form a phosphonate group —O—P(═O)(OH)₂. Such a group can be cleaved by phosphatase enzymes during metabolism to yield the active drug with the hydroxy group.

Also, some prodrugs are activated enzymatically to yield the active compound, or a compound which, upon further chemical reaction, yields the active compound. For example, the prodrug may be a sugar derivative or other glycoside conjugate, or may be an amino acid ester derivative.

### Acronyms

For convenience, many chemical moieties are represented using well known abbreviations, including but not limited to, methyl (Me), ethyl (Et), n-propyl (nPr), iso-propyl (iPr), n-butyl (nBu), tert-butyl (tBu), n-hexyl (nHex), cyclohexyl (cHex), phenyl (Ph), biphenyl (biPh), benzyl (Bn), naphthyl (naph), methoxy (MeO), ethoxy (EtO), benzoyl (Bz), and acetyl (Ac).

For convenience, many chemical compounds are represented using well known abbreviations, including but not limited to, methanol (MeOH), ethanol (EtOH), iso-propanol (i-PrOH), methyl ethyl ketone (MEK), ether or diethyl ether (Et₂O), acetic acid (AcOH), dichloromethane (methylene chloride, DCM), trifluoroacetic acid (TFA), dimethylformamide (DMF), tetrahydrofuran (THF), and dimethylsulfoxide (DMSO).

### Synthesis

Compounds of formula (I):

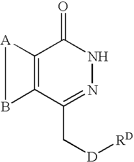

may in general be synthesised by the coupling of a compound of formula 2:

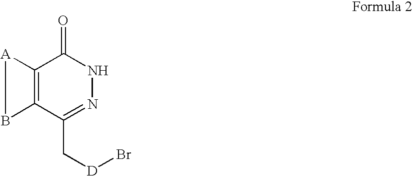

with the appropriate arylboronic acid, or equivalent. This Suzuki coupling may be carried out using conventional reagents, such as potassium carbonate and tetrakis (triphenylphosphone) palladium in a suitable organic solvent.

For some compounds, a precursor to R^{D} may be introduced, before the desired substitution on R^{D} is introduced. The precursor may be protected accordingly. In particular, if the substitution on R^{D} is an amido group, then a carboxy precursor may be coupled, and if the substitution on R^{D} is an acylamido, sulfonamido or ureido group, then an amino precursor may be coupled.

Compounds of formula 2 may be synthesised from compounds of formula 3:

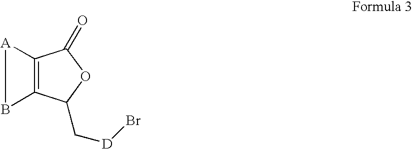

by reaction with hydrazine hydrate in a water suspension.

Compounds of formula 3 may be coupling compounds of formulae 4 and 5:

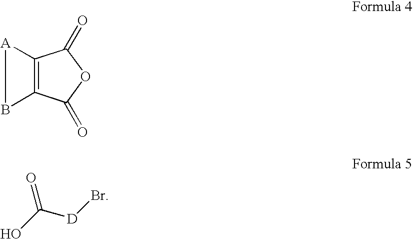

Other routes to compounds of formula 2 are known.

### Use

The present invention provides active compounds, specifically, active in inhibiting the activity of PARP-1.

The term “active” as used herein, pertains to compounds which are capable of inhibiting PARP-1 activity, and specifically includes both compounds with intrinsic activity (drugs) as well as prodrugs of such compounds, which prodrugs may themselves exhibit little or no intrinsic activity.

One assay which may conveniently be used in order to assess the PARP-1 inhibition offered by a particular compound is described in the examples below.

The present invention further provides a method of inhibiting the activity of PARP-1 in a cell, comprising contacting said cell with an effective amount of an active compound, preferably in the form of a pharmaceutically acceptable composition. Such a method may be practised in vitro or in vivo.

For example, a sample of cells may be grown in vitro and an active compound brought into contact with said cells, and the effect of the compound on those cells observed. As examples of “effect”, the amount of DNA repair effected in a certain time may be determined. Where the active compound is found to exert an influence on the cells, this may be used as a prognostic or diagnostic marker of the efficacy of the compound in methods of treating a patient carrying cells of the same cellular type.

The term “treatment”, as used herein in the context of treating a condition, pertains generally to treatment and therapy, whether of a human or an animal (e.g. in veterinary applications), in which some desired therapeutic effect is achieved, for example, the inhibition of the progress of the condition, and includes a reduction in the rate of progress, a halt in the rate of progress, amelioration of the condition, and cure of the condition. Treatment as a prophylactic measure (i.e. prophylaxis) is also included.

The term “adjunct” as used herein relates to the use of active compounds in conjunction with known therapeutic means. Such means include cytotoxic regimens of drugs and/or ionising radiation as used in the treatment of different cancer types. In particular, the active compounds are known to potentiate the actions of a number of cancer chemotherapy treatments, which include the topoisomerase class of poisons and most of the known alkylating agents used in treating cancer.

Active compounds may also be used as cell culture additives to inhibit PARP, for example, in order to sensitize cells to known chemotherapeutic agents or ionising radiation treatments in vitro.

Active compounds may also be used as part of an in vitro assay, for example, in order to determine whether a candidate host is likely to benefit from treatment with the compound in question.

### Administration

The active compound or pharmaceutical composition comprising the active compound may be administered to a subject by any convenient route of administration, whether systemically/peripherally or at the site of desired action, including but not limited to, oral (e.g. by ingestion); topical (including e.g. transdermal, intranasal, ocular, buccal, and sublingual); pulmonary (e.g. by inhalation or insufflation therapy using, e.g. an aerosol, e.g. through mouth or nose); rectal; vaginal; parenteral, for example, by injection, including subcutaneous, intradermal, intramuscular, intravenous, intraarterial, intracardiac, intrathecal, intraspinal, intracapsular, subcapsular, intraorbital, intraperitoneal, intratracheal, subcuticular, intraarticular, subarachnoid, and intrasternal; by implant of a depot, for example, subcutaneously or intramuscularly.

The subject may be a eukaryote, an animal, a vertebrate animal, a mammal, a rodent (e.g. a guinea pig, a hamster, a rat, a mouse), murine (e.g. a mouse), canine (e.g. a dog), feline (e.g. a cat), equine (e.g. a horse), a primate, simian (e.g. a monkey or ape), a monkey (e.g. marmoset, baboon), an ape (e.g. gorilla, chimpanzee, orang-utan, gibbon), or a human.

### Formulations

While it is possible for the active compound to be administered alone, it is preferable to present it as a pharmaceutical composition (e.g., formulation) comprising at least one active compound, as defined above, together with one or more pharmaceutically acceptable carriers, adjuvants, excipients, diluents, fillers, buffers, stabilisers, preservatives, lubricants, or other materials well known to those skilled in the art and optionally other therapeutic or prophylactic agents.

Thus, the present invention further provides pharmaceutical compositions, as defined above, and methods of making a pharmaceutical composition comprising admixing at least one active compound, as defined above, together with one or more pharmaceutically acceptable carriers, excipients, buffers, adjuvants, stabilisers, or other materials, as described herein.

The term “pharmaceutically acceptable” as used herein pertains to compounds, materials, compositions, and/or dosage forms which are, within the scope of sound medical judgement, suitable for use in contact with the tissues of a subject (e.g. human) without excessive toxicity, irritation, allergic response, or other problem or complication, commensurate with a reasonable benefit/risk ratio. Each carrier, excipient, etc. must also be “acceptable” in the sense of being compatible with the other ingredients of the formulation.

Suitable carriers, diluents, excipients, etc. can be found in standard pharmaceutical texts. See, for example, “Handbook of Pharmaceutical Additives”, 2nd Edition (eds. M. Ash and I. Ash), 2001 (Synapse Information Resources, Inc., Endicott, N.Y., USA), “Remington's Pharmaceutical Sciences”, 20th edition, pub. Lippincott, Williams & Wilkins, 2000; and “Handbook of Pharmaceutical Excipients”, 2nd edition, 1994.

The formulations may conveniently be presented in unit dosage form and may be prepared by any methods well known in the art of pharmacy. Such methods include the step of bringing into association the active compound with the carrier which constitutes one or more accessory ingredients. In general, the formulations are prepared by uniformly and intimately bringing into association the active compound with liquid carriers or finely divided solid carriers or both, and then if necessary shaping the product.

Formulations may be in the form of liquids, solutions, suspensions, emulsions, elixirs, syrups, tablets, lozenges, granules, powders, capsules, cachets, pills, ampoules, suppositories, pessaries, ointments, gels, pastes, creams, sprays, mists, foams, lotions, oils, boluses, electuaries, or aerosols.

Formulations suitable for oral administration (e.g., by ingestion) may be presented as discrete units such as capsules, cachets or tablets, each containing a predetermined amount of the active compound; as a powder or granules; as a solution or suspension in an aqueous or non-aqueous liquid; or as an oil-in-water liquid emulsion or a water-in-oil liquid emulsion; as a bolus; as an electuary; or as a paste.

A tablet may be made by conventional means, e.g. compression or moulding, optionally with one or more accessory ingredients. Compressed tablets may be prepared by compressing in a suitable machine the active compound in a free-flowing form such as a powder or granules, optionally mixed with one or more binders (e.g. povidone, gelatin, acacia, sorbitol, tragacanth, hydroxypropylmethyl cellulose); fillers or diluents (e.g. lactose, microcrystalline cellulose, calcium hydrogen phosphate); lubricants (e.g. magnesium stearate, talc, silica); disintegrants (e.g. sodium starch glycolate, cross-linked povidone, cross-linked sodium carboxymethyl cellulose); surface-active or dispersing or wetting agents (e.g., sodium lauryl sulfate); and preservatives (e.g., methyl p-hydroxybenzoate, propyl p-hydroxybenzoate, sorbic acid). Moulded tablets may be made by moulding in a suitable machine a mixture of the powdered compound moistened with an inert liquid diluent. The tablets may optionally be coated or scored and may be formulated so as to provide slow or controlled release of the active compound therein using, for example, hydroxypropylmethyl cellulose in varying proportions to provide the desired release profile. Tablets may optionally be provided with an enteric coating, to provide release in parts of the gut other than the stomach.

Formulations suitable for topical administration (e.g. transdermal, intranasal, ocular, buccal, and sublingual) may be formulated as an ointment, cream, suspension, lotion, powder, solution, past, gel, spray, aerosol, or oil. Alternatively, a formulation may comprise a patch or a dressing such as a bandage or adhesive plaster impregnated with active compounds and optionally one or more excipients or diluents.

Formulations suitable for topical administration in the mouth include losenges comprising the active compound in a flavored basis, usually sucrose and acacia or tragacanth; pastilles comprising the active compound in an inert basis such as gelatin and glycerin, or sucrose and acacia; and mouthwashes comprising the active compound in a suitable liquid carrier.

Formulations suitable for topical administration to the eye also include eye drops wherein the active compound is dissolved or suspended in a suitable carrier, especially an aqueous solvent for the active compound.

Formulations suitable for nasal administration, wherein the carrier is a solid, include a coarse powder having a particle size, for example, in the range of about 20 to about 500 microns which is administered in the manner in which snuff is taken, i.e. by rapid inhalation through the nasal passage from a container of the powder held close up to the nose. Suitable formulations wherein the carrier is a liquid for administration as, for example, nasal spray, nasal drops, or by aerosol administration by nebuliser, include aqueous or oily solutions of the active compound.

Formulations suitable for administration by inhalation include those presented as an aerosol spray from a pressurised pack, with the use of a suitable propellant, such as dichlorodifluoromethane, trichlorofluoromethane, dichoro-tetrafluoroethane, carbon dioxide, or other suitable gases.

Formulations suitable for topical administration via the skin include ointments, creams, and emulsions. When formulated in an ointment, the active compound may optionally be employed with either a paraffinic or a water-miscible ointment base. Alternatively, the active compounds may be formulated in a cream with an oil-in-water cream base. If desired, the aqueous phase of the cream base may include, for example at least about 30% w/w of a polyhydric alcohol, i.e., an alcohol having two or more hydroxyl groups such as propylene glycol, butane-1,3-diol, mannitol, sorbitol, glycerol and polyethylene glycol and mixtures thereof. The topical formulations may desirably include a compound which enhances absorption or penetration of the active compound through the skin or other affected areas. Examples of such dermal penetration enhancers include dimethylsulfoxide and related analogues.

When formulated as a topical emulsion, the oily phase may optionally comprise merely an emulsifier (otherwise known as an emulgent), or it may comprises a mixture of at least one emulsifier with a fat or an oil or with both a fat and an oil. Preferably, a hydrophilic emulsifier is included together with a lipophilic emulsifier which acts as a stabiliser. It is also preferred to include both an oil and a fat. Together, the emulsifier(s) with or without stabiliser(s) make up the so-called emulsifying wax, and the wax together with the oil and/or fat make up the so-called emulsifying ointment base which forms the oily dispersed phase of the cream formulations.

Suitable emulgents and emulsion stabilisers include Tween 60, Span 80, cetostearyl alcohol, myristyl alcohol, glyceryl monostearate and sodium lauryl sulphate. The choice of suitable oils or fats for the formulation is based on achieving the desired cosmetic properties, since the solubility of the active compound in most oils likely to be used in pharmaceutical emulsion formulations may be very low. Thus the cream should preferably be a non-greasy, non-staining and washable product with suitable consistency to avoid leakage from tubes or other containers. Straight or branched chain, mono- or dibasic alkyl esters such as di-isoadipate, isocetyl stearate, propylene glycol diester of coconut fatty acids, isopropyl myristate, decyl oleate, isopropyl palmitate, butyl stearate, 2-ethylhexyl palmitate or a blend of branched chain esters known as Crodamol CAP may be used, the last three being preferred esters. These may be used alone or in combination depending on the properties required. Alternatively, high melting point lipids such as white soft paraffin and/or liquid paraffin or other mineral oils can be used.

Formulations suitable for rectal administration may be presented as a suppository with a suitable base comprising, for example, cocoa butter or a salicylate.

Formulations suitable for vaginal administration may be presented as pessaries, tampons, creams, gels, pastes, foams or spray formulations containing in addition to the active compound, such carriers as are known in the art to be appropriate.

Formulations suitable for parenteral administration (e.g., by injection, including cutaneous, subcutaneous, intramuscular, intravenous and intradermal), include aqueous and non-aqueous isotonic, pyrogen-free, sterile injection solutions which may contain anti-oxidants, buffers, preservatives, stabilisers, bacteriostats, and solutes which render the formulation isotonic with the blood of the intended recipient; and aqueous and non-aqueous sterile suspensions which may include suspending agents and thickening agents, and liposomes or other microparticulate systems which are designed to target the compound to blood components or one or more organs. Examples of suitable isotonic vehicles for use in such formulations include Sodium Chloride Injection, Ringer's Solution, or Lactated Ringer's Injection. Typically, the concentration of the active compound in the solution is from about 1 ng/ml to about 10 μg/ml, for example from about 10 ng/ml to about 1 μg/ml. The formulations may be presented in unit-dose or multi-dose sealed containers, for example, ampoules and vials, and may be stored in a freeze-dried (lyophilised) condition requiring only the addition of the sterile liquid carrier, for example water for injections, immediately prior to use. Extemporaneous injection solutions and suspensions may be prepared from sterile powders, granules, and tablets. Formulations may be in the form of liposomes or other microparticulate systems which are designed to target the active compound to blood components or one or more organs.

### Dosage

It will be appreciated that appropriate dosages of the active compounds, and compositions comprising the active compounds, can vary from patient to patient. Determining the optimal dosage will generally involve the balancing of the level of therapeutic benefit against any risk or deleterious side effects of the treatments of the present invention. The selected dosage level will depend on a variety of factors including, but not limited to, the activity of the particular compound, the route of administration, the time of administration, the rate of excretion of the compound, the duration of the treatment, other drugs, compounds, and/or materials used in combination, and the age, sex, weight, condition, general health, and prior medical history of the patient. The amount of compound and route of administration will ultimately be at the discretion of the physician, although generally the dosage will be to achieve local concentrations at the site of action which achieve the desired effect without causing substantial harmful or deleterious side-effects.

Administration in vivo can be effected in one dose, continuously or intermittently (e.g., in divided doses at appropriate intervals) throughout the course of treatment. Methods of determining the most effective means and dosage of administration are well known to those of skill in the art and will vary with the formulation used for therapy, the purpose of the therapy, the target cell being treated, and the subject being treated. Single or multiple administrations can be carried out with the dose level and pattern being selected by the treating physician.

In general, a suitable dose of the active compound is in the range of about 100 μg to about 250 mg per kilogram body weight of the subject per day. Where the active compound is a salt, an ester, prodrug, or the like, the amount administered is calculated on the basis of the parent compound and so the actual weight to be used is increased proportionately.

### EXAMPLES

### General Methods

### HPLC

### Method 1

Instrument: Finnegan LCQ in positive ion mode
Mobile Phase A: 0.1% Formic acid in water

### Mobile Phase B: Acetonitrile

### Column: Jones Chromatography Genesis C18 4 μm 50×4.6 mm

Gradient: Starting at 95% A/5% B for one minute, rising to 98% B after 5 minutes, holding for 3 minutes, then back to starting conditions
Flow rate: 2.0 ml/min.
PDA Scan range: 254 nm.

### Method 2

Instrument: Waters ZQ LC-MS system No. LAA 254 operating in Electrospray ionisation mode.
Mobile Phase A: 0.1% Formic acid in water
Mobile Phase B: 0.1% Formic acid in acetonitrile

### Column: Genesis C18 4 μm 50×4.6 mm

### Gradient:

| Time (mins.) | % B |
|---|---|
| 0 | 5 |
| 5 | 95 |
| 10 | 95 |
| 10.5 | 5 |
| 11 | 5 |

Flow rate: 2.0 ml/min.
PDA Scan range: 210-400 nm.

### Method 3

Instrument: Waters ZQ LC-MS system No. LAA 254 operating in Electrospray ionisation mode.
Mobile Phase A: 0.1% Formic acid in water
Mobile Phase B: 0.1% Formic acid in acetonitrile

Column: Genesis C18 4 μm 50×4.6 mm

### Gradient:

| Time (mins.) | % B |
|---|---|
| 1 | 5 |
| 20 | 95 |
| 23 | 95 |
| 24 | 5 |
| 25 | 5 |

Flow rate: 2.0 ml/min.
PDA Scan range: 210-400 nm.

### Method 4

Instrument: Waters ZQ LC-MS system No. LAA 254 operating in Electrospray ionisation mode.
Mobile Phase A: 0.1% Formic acid in water
Mobile Phase B: 0.1% Formic acid in acetonitrile

### Column: Genesis C18 4 μm 50×4.6 mm

| Time (mins.) | % B |
|---|---|
| 0 | 5 |
| 5 | 95 |
| 10 | 95 |
| 10.5 | 5 |
| 11 | 5 |

Flow rate: 2.0 ml/min.
PDA Scan range: 210-400 nm.

### Example 1

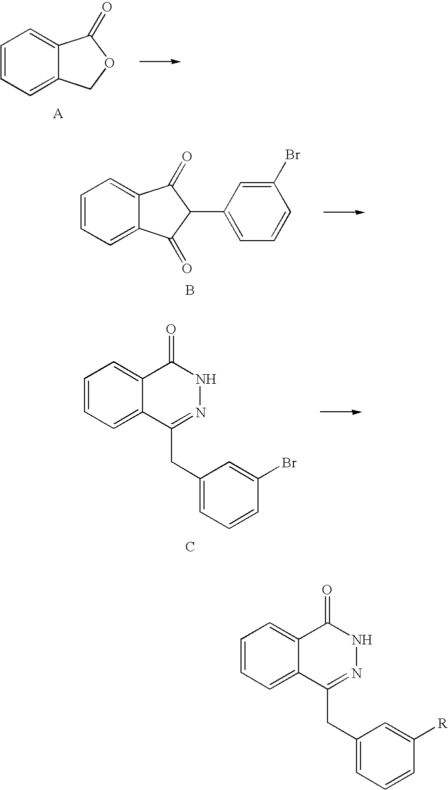

### (a) 2-(3-Bromo-phenyl)-indan-1,3-dione (B)

Phthalide (A)(13.4 g, 100.0 mmol) and 3-bromobenzaldehyde (18.5 g, 100.0 mmol) were added to a preformed solution of sodium methoxide (21.6 g, 100.0 mmol) in methanol (95 mL) and ethyl propionate (50 mL) over 40 minutes. The resulting orange solution was then heated to 72° C. for 2 hours before being cooled to room temperature and diluted with ice-cold water (400 mL). The aqueous phase was washed with ether (5×150 mL) and treated with glacial acetic acid (12 mL). The mixture cooled to 0° C. and the red precipitate filtered. The solid was then washed with water (2×10 mL) and then dried in vacuum oven overnight. Single peak in LC-MS, (24.9 g, 99% purity) and required no further purification; m/z (LC-MS, ESP), RT=4.51 mins (M+H) 301.1, 303.1;

### (b) 4-(3-Bromo-benzyl)-2H-phthalazin-1-one (C)

2-(3-Bromo-phenyl)-indan-1,3-dione (B)(3.01 g, 10.0 mmol) was suspended in hydrazine hydrate (15 mL, 0.31 mol) and heated to 100° C. for 18 hours. The mixture was then cooled to 0° C. and the cream precipitate filtered and washed with water (2×20 ml) before being dried in vacuo to afford a cream solid. Single peak in LC-MS, (2.56 g, 99% purity) and required no further purification; m/z (LC-MS, ESP), RT=3.70 mins (M+H) 316.7.

### (c) Library Synthesis

To 4-(3-bromo-benzyl)-2H-phthalazin-1-one (C)(0.050 g, 0.16 mmol) dissolved in dioxane (1 mL) was added boronic acid (0.190 mmol), potassium carbonate (48 mg, 0.34 mmol) and tetrakis(triphenylphosphine)palladium (9 mg, 0.01 mmol). The reaction was degassed by bubbling nitrogen into the mixture in conjunction with sonic agitation for 10 minutes before being heated to 80° C. for 18 hours under nitrogen. The reaction was then cooled to room temperature and passed through a plug of silica gel (0.5 g), eluting with 70:30 dichloromethane:methanol (5 mL). The resultant solution was then concentrated and submitted for preparative HPLC purification.

The compounds synthesised are set out below:

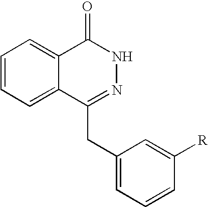

| | | RT (mins) | | Purity |
|---|---|---|---|---|
| Compound | R | Method 1 | M + H | (%) |
| 1 | 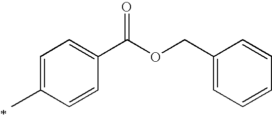 | 4.09 | 447.1 | >85 |
| 2 | 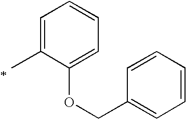 | 4.89 | 419.1 | >85 |
| 3 | 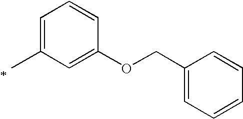 | 4.95 | 419.1 | >85 |
| 4 | 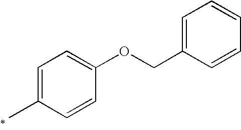 | 5.01 | 419.2 | >85 |
| 5 | 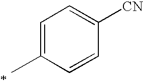 | 4.22 | 338.3 | >85 |
| 6 | 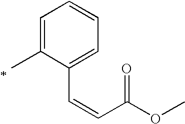 | 4.60 | 397.1 | >85 |
| 7 | 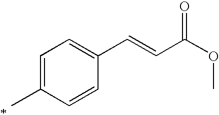 | 4.58 | 397.1 | >85 |
| 8 | 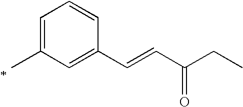 | 4.56 | 397.1 | >85 |
| 9 | 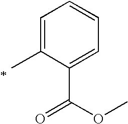 | 4.29 | 371.1 | >85 |
| 10 | 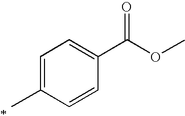 | 4.49 | 371.1 | >85 |
| 11 | 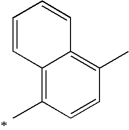 | 5.07 | 377.2 | >85 |
| 12 | 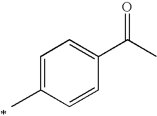 | 4.19 | 355.2 | >85 |
| 13 | 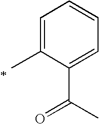 | 4.13 | 355.2 | >85 |
| 14 | 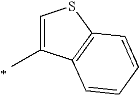 | 4.79 | 369.2 | >85 |
| 15 | 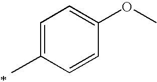 | 4.36 | 343.2 | >85 |
| 16 | 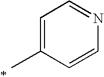 | 2.95 | 314.4 | >85 |
| 17 | 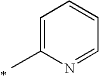 | 3.24 | 314.3 | >85 |
| 18 | 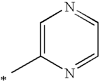 | 3.51 | 315.3 | >85 |
| 19 | 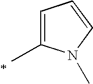 | 4.18 | 316.3 | >85 |
| 20 | 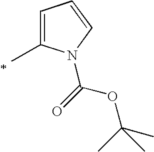 | 4.75 | 402.0 | >85 |
| 21 | 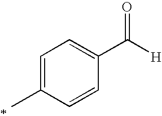 | 4.17 | 341.2 | >85 |
| 22 | 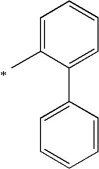 | 4.92 | 389.3 | >85 |
| 23 | 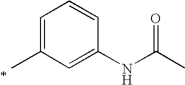 | 3.73 | 370.2 | >85 |
| 24 | 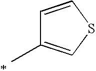 | 4.34 | 319.2 | >85 |
| 25 | 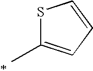 | 4.42 | 319.3 | >85 |
| 26 | 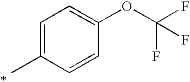 | 4.88 | 397.2 | >85 |
| 27 | 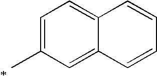 | 4.87 | 363.3 | >85 |
| 28 | 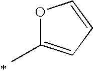 | 4.19 | 303.2 | >85 |
| 29 | 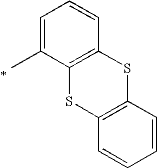 | 5.23 | 4551.2 | >85 |
| 30 | 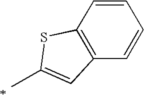 | 4.89 | 369.2 | >85 |
| 31 | 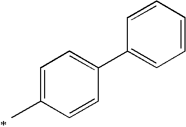 | 5.01 | 389.3 | >85 |
| 32 | 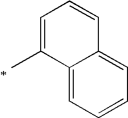 | 4.89 | 363.3 | >85 |
| 33 | 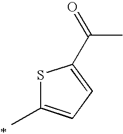 | 4.07 | 361.2 | >85 |
| 34 | 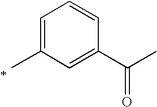 | 4.12 | 355.1 | >85 |
| 35 | 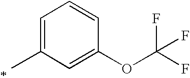 | 4.90 | 397.2 | >85 |
| 36 | 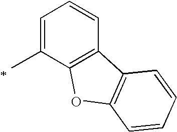 | 5.08 | 403.3 | >85 |
| 37 | 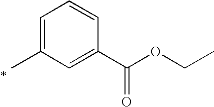 | 4.79 | 385.1 | >85 |
| 38 | 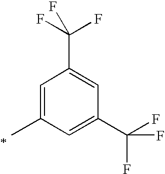 | 5.08 | 449.2 | >85 |
| 39 | 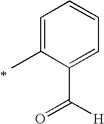 | 4.20 | 341.1 | >85 |
| 40 | 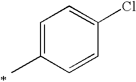 | 4.79 | 347.2 | >85 |
| 41 | 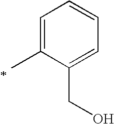 | 3.84 | 343.0 | >85 |
| 42 | 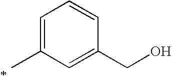 | 3.77 | 343.0 | >85 |
| 43 | 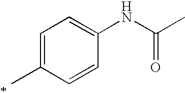 | 3.67 | 370.2 | >85 |
| 44 | 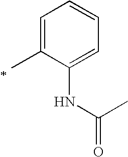 | 3.70 | 370.1 | >85 |
| 45 | 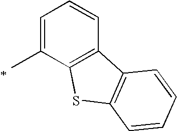 | 5.15 | 419.2 | >85 |
| 46 | 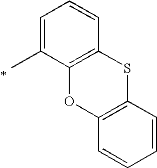 | 5.15 | 435.2 | >85 |
| 47 | 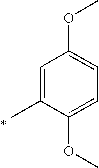 | 4.33 | 373.2 | >85 |
| 48 | 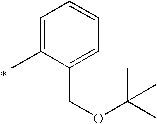 | 4.93 | 389.9 | >85 |

### Example 2

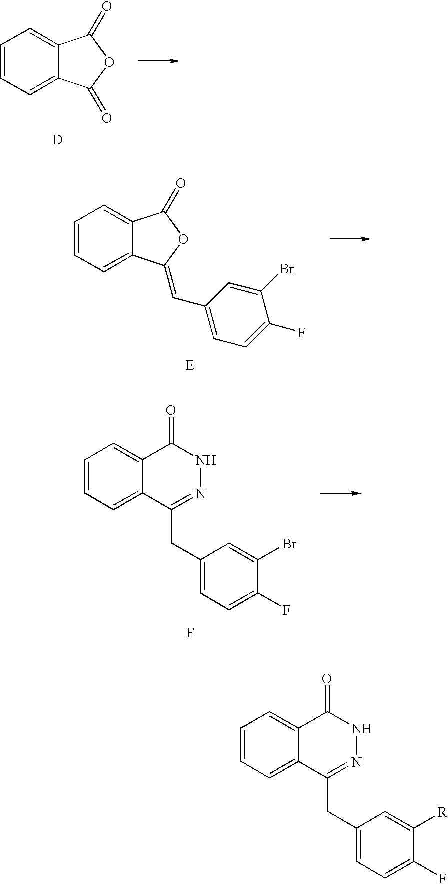

### (a) 3-(3-Bromo-4-fluoro-benzylidene)-3H-isobenzofuran-1-one (E)

Phthalic anhydride (D) (0.64 g, 4.290 mmol), 3-bromo-4-fluorophenylacetic acid (1.0 g, 4.29 mmol) and sodium acetate (0.017 g, 0.0204 mmol) were fused together at 240-245° C. for 25 minutes. The reaction was cool gradually over 30 minutes to 90° C. Ethanol (7 mL) was cautiously added to the reaction mixture and the resultant precipitate filtered and washed with cold ethanol (1×5 ml) before being dried in vacuo. Single peak in LC-MS, (1.04 g, 95% purity) and required no further purification; m/z (LC-MS, ESP), RT=4.71 mins (M+H) 319.1 & 321.1.

### (b) 4-(3-Bromo-4-fluoro-benzyl)-2H-phthalazin-1-one (F)

Hydrazine hydrate (0.175 g, 3.5 mmol) was added to 3-(3-Bromo-4-fluoro-benzylidene)-3H-isobenzofuran-1-one (E)(0.319 g, 1.0 mmol) suspended in water (30 ml) over 2 minutes. The solution was then heated to 100° C. for 18 hours. The mixture was allowed to cool to 0° C. and diluted with HCl (2N, 1 mL). The precipitate was filtered, washed with water (2×10 ml) and dried in vacuo to afford a beige solid. Single peak in LC-MS, (0.28 g, 95% purity) and required no further purification; m/z (LC-MS, ESP), RT=3.77 mins (M+H) 333.1 & 335.1;

### (c) Library Synthesis

To 4-(3-bromo-4-fluoro-benzyl)-2H-phthalazin-1-one (F)(0.025 g, 0.075 mmol) dissolved in dioxane (1 mL), was added boronic acid (0.226 mmol), potassium carbonate (29 mg, 0.210 mmol) and tetrakis (triphenylphosphine) palladium (4 mg, 0.003 mmol). The reaction vessel was purged with nitrogen and subjected to sonic agitation for 10 minutes, before being irradiated by microwave, maintaining the reaction at a fixed temperature of 120° C. for 30 minutes. The resultant mixture was then passed through a plug of silica gel (0.5 g), eluting with 70:30 dichloromethane:methanol (5 mL). The filtrate was concentrated in vacuo and crude material submitted for prep HPLC purification.

The compounds synthesised are set out below:

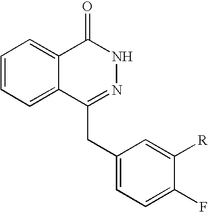

| | | RT (mins) | | Purity |
|---|---|---|---|---|
| Compound | R | Method 2 | M + H | (%) |
| 49 | 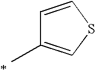 | 5.54 | 337.2 | 100 |
| 50 | 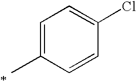 | 6.02 | 365.2 | 97 |
| 51 | 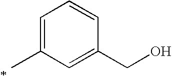 | 4.814 | 361.3 | 98 |
| 52 | 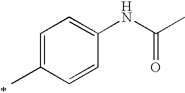 | 4.75 | 388.3 | 91 |
| 53 | 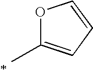 | 5.42 | 321.3 | 98 |
| 54 | 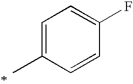 | 5.81 | 349.3 | 89 |
| 55 | 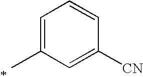 | 5.52 | 356.4 | 85 |
| 56 | 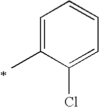 | 5.80 | 365.3 | 99 |
| 57 | 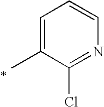 | 10.21* | 366.3 | 85 |
| 58 | 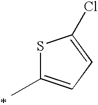 | 6.28 | 371.2 | 99 |
| 59 | 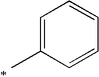 | 5.61 | 331.2 | 99 |
| 60 | 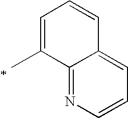 | 4.84 | 382.2 | 98 |
| 61 | 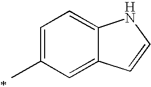 | 5.31 | 370.2 | 90 |
| 62 | 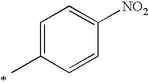 | 5.59 | 376.2 | 96 |
| 63 | 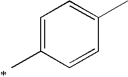 | 5.92 | 345.2 | 98 |
| 64 | 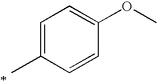 | 5.58 | 361.2 | 95 |
| 65 | 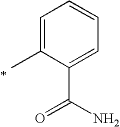 | 8.47* | 374.2 | 87 |
| 66 | 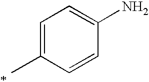 | 8.17* | 346.2 | 96 |
| 67 | 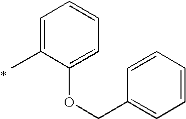 | 14.17* | 437.3 | 97 |
| 68 | 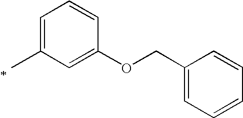 | 14.84* | 437.3 | 99 |
| 69 | 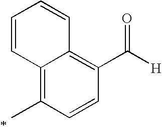 | 12.74* | 409 | 86 |
| 70 | 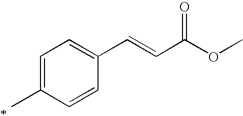 | 13.03* | 415.3 | 97 |
| 71 | 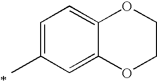 | 12.06* | 389.3 | 84 |
| 72 | 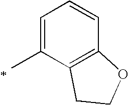 | 12.25* | 373.3 | 88 |
| 73 | 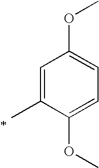 | 11.80* | 391.3 | 87 |
| 74 | 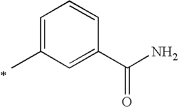 | 9.07* | 374.2 | 96 |
| 75 | 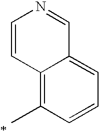 | 7.78* | 382.2 | 99 |
| 76 | 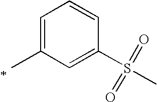 | 10.31* | 409.2 | 77 |
| 77 | 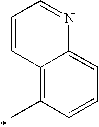 | 8.43* | 382.2 | 98 |
| 78 | 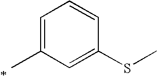 | 13.33* | 377.2 | 97 |
| 79 | 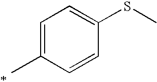 | 13.42* | 377.2 | 96 |
| 80 | 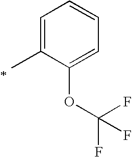 | 13.28* | 415.2 | 97 |
| 81 | 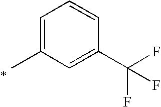 | 13.87* | 399.2 | 99 |
| 82 | 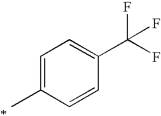 | 14.07* | 399.2 | 98 |
| 83 | 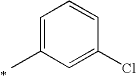 | 13.57* | 365.1 | 97 |
| 85 | 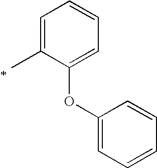 | 6.19 | 423.2 | 92 |
| 86 | 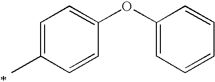 | 6.52 | 423.2 | 99 |
| 87 | 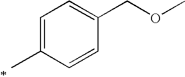 | 5.63 | 375.2 | 97 |
| 88 | 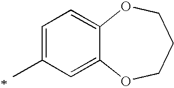 | 5.73 | 403.2 | 98 |
| 89 | 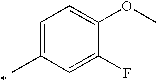 | 5.73 | 379.2 | 99 |
| 90 | 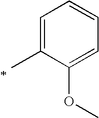 | 5.60 | 361.2 | 96 |
| 91 | 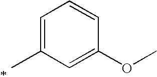 | 5.72 | 361.2 | 98 |
| 92 | 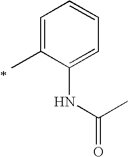 | 4.74 | 388.2 | 99 |
| 93 | 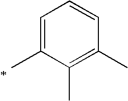 | 6.09 | 359.3 | 99 |
| 94 | 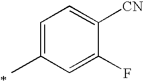 | 12.13* | 374.3 | 92 |
| 95 | 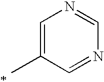 | 6.04 | 384.3 | 94 |
| 96 | 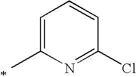 | 13.50* | 398.3 | 89 |

*indicates HPLC Method 3

### Example 3

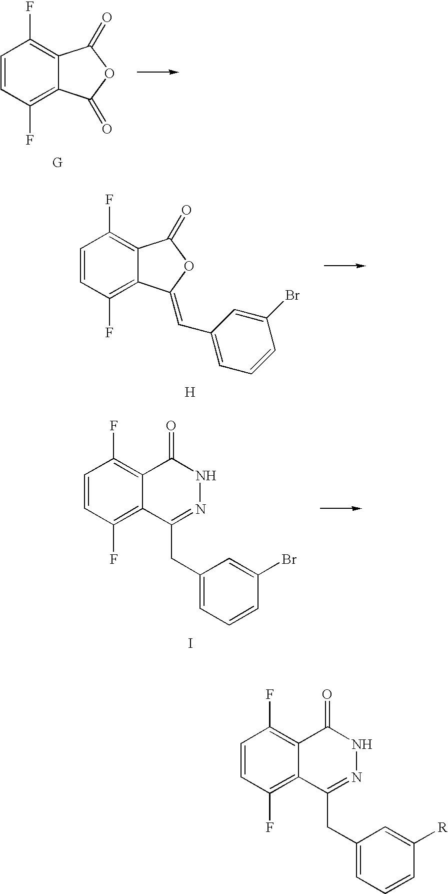

### (a) 3-(3-Bromo-benzylidene)-4,7-difluoro-3H-isobenzofuran-1-one (H)

3,6 Difluoro phthalic anhydride (G) (6.42 g, 34.87 mmol), 3-bromo-phenylacetic acid (5.0 g, 23.2 mmol) and sodium acetate (0.09 g, 1.1 mmol) were fused together at 250-255° C. for 20 minutes. The reaction was cooled gradually over 1 hour to approximately 90° C. Ethanol (25 mL) was added cautiously to the reaction mixture and the resultant precipitate filtered and washed with cold ethanol (1×15 ml). The orange solid was then dried in vacuo. Single peak in LC-MS, (5.29 g, 95% purity) and required no further purification; m/z (LC-MS, ESP), RT=4.76 mins (M+NH₄) 353.1 & 355.1.

### (b) 4-(3-Bromo-benzyl)-5,8-difluoro-2H-phthalazin-1-one (I)

Hydrazine monohydrate (2.87 mL, 59.31 mmol) was added to 3-(3-bromo-benzylidene)-4,7-difluoro-3H-isobenzofuran-1-one (H) (5.29 g, 15.7 mmol), suspended in water (100 mL) and then heated to 100° C. for 18 hours. The mixture was cooled to 0° C. and diluted with HCl (2N, 5 mL). The precipitate was filtered, washed with water (2×20 ml) and dried in vacuo to afford a yellow solid. Single peak in LC-MS, (3.78 g, 95% purity) and required no further purification; m/z (LC-MS, ESP), RT=3.50 mins (M+H₂O) 368.1 & 370.1.

### (c) Library Synthesis

To 4-(3-bromo-benzyl)-5,8-difluoro-2H-phthalazin-1-one (I) (0.026 g, 0.075 mmol) dissolved in dioxane (1 mL), was added boronic acid (0.226 mmol), potassium carbonate (29 mg, 0.210 mmol) and tetrakis (triphenylphosphine) palladium (4 mg, 0.003 mmol). The reaction vessel was purged with nitrogen and subjected to sonic agitation to remove dissolved gasses for 5 minutes, before being irradiated by microwave, maintaining the reaction vessel at a fixed temperature of 120° C. for 30 minutes. The resultant mixture was passed through a plug of silica gel (0.5 g) eluting with 70:30 dichloromethane:methanol (5 mL). The filtrate was concentrated in vacuo and crude material subjected to prep purification.

The compounds synthesised are set out below:

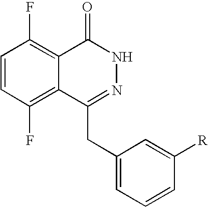

| Com- | | RT (mins) | | Purity |
|---|---|---|---|---|
| pound | R | Method 2 | M + H | (%) |
| 97 | 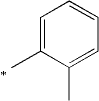 | 6.08 | 363.3 | 94 |
| 98 | 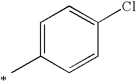 | 6.22* | 383.2 | 96 |
| 99 | 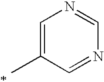 | 4.97 | 351.2 | 94 |
| 100 | 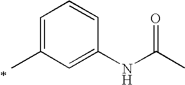 | 5.89 | 367.2 | 93 |
| 101 | 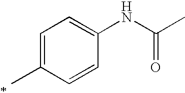 | 5.00 | 406.4 | 88 |
| 102 | 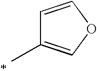 | 5.46 | 339.3 | 96 |
| 103 | 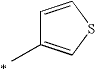 | 5.72 | 355.3 | 93 |
| 104 | 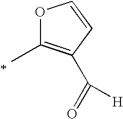 | 10.45* | 367.3 | 85 |
| 105 | 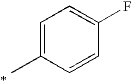 | 5.90 | 367.4 | 98 |

*indicates HPLC Method 3

### Example 4

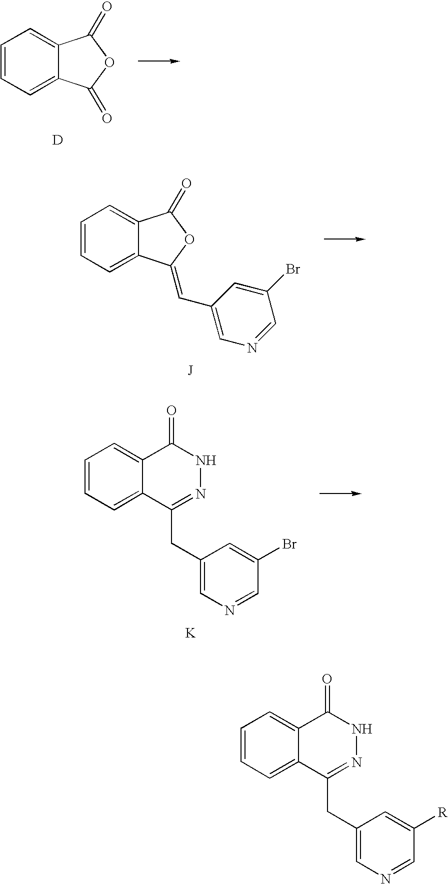

### (a) 3-(5-Bromo-pyridin-3-ylmethylene)-3H-isobenzofuran-1-one (J)

5-Bromo-pyridylacetic acid (0.495 g, 2.290 mmol), phthalic anhydride (D) (0.340 g, 2.29 mmol) and sodium acetate (10 mg, 0.115 mmol) were heated neat at 230° C. using a wood's alloy bath for 70 minutes. The melt was then cooled to 90° C. and diluted with ethanol (10 mL). The resultant solid was slurried in the ethanol for 15 minutes and then isolated by filtration. Main peak in LC-MS, (0.575 g, 85% purity) taken through crude without need for any purification; m/z (LC-MS, ESP), RT=4.20 mins (M+H) 302.2 & 304.3.

### (b) 4-(5-Bromo-pyridin-3-ylmethyl)-2H-phthalazin-1-one (K)

3-(5-Bromo-pyridin-3-ylmethylene)-3H-isobenzofuran-1-one (J) (0.574 g 1.9 mmol) was suspended in water (10 mL) and heated to 50° C. After 30 minutes hydrazine hydrate (1 mL, 3.84 mmol) was added dropwise to the solution and then temperature elevated to 100° C. for 1 hour. The reaction was then cooled to room temperature and concentrated to dryness and absorbed onto silica gel. The material as then subjected to flash chromatography eluent ethyl acetate/hexane 1:1 (rf of 0.34). The title compound was isolated as a beige solid. Single peak in LC-MS, (0.30 g, 100% purity); m/z (LC-MS, ESP), RT=3.15 mins (M+H) 317.2.

### (c) Library Synthesis

To 4-(5-bromo-pyridin-3-ylmethyl)-2H-phthalazin-1-one (K) (0.016 g, 0.050 mmol) dissolved in acetonitrile (500 μL) and water (500 μL) was added boronic acid (0.075 mmol), potassium carbonate (21 mg, 0.150 mmol) and tetrakis (triphenylphosphine) palladium (4 mg, 0.003 mmol). The reaction vessel was purged with nitrogen and subjected to sonic agitation for 5 minutes, before being irradiated by microwave, maintaining the reaction vessel at a fixed temperature of 120° C. for 30 minutes. The resultant mixture was passed through a plug of silica gel (0.5 g) eluting with 70:30 dichloromethane:methanol (5 mL). The filtrate was concentrated in vacuo and crude material subjected to prep purification.

The compounds synthesised are set out below:

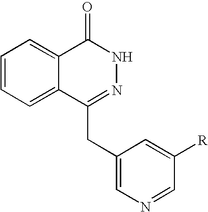

| | | RT | | |
|---|---|---|---|---|
| | | (mins) | | Pu- |
| Com- | | Method | M + | rity |
| pound | R | 2 | H | (%) |
| 106 | 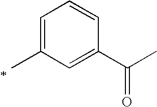 | 4.01 | 356.3 | 99 |
| 107 | 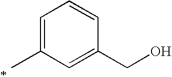 | 3.59 | 344.3 | 100 |
| 108 | 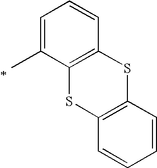 | 5.40 | 452.3 | 95 |
| 109 | 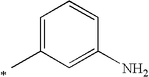 | 3.32 | 329.3 | 98 |
| 110 | 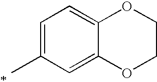 | 4.00 | 372.3 | 99 |
| 111 | 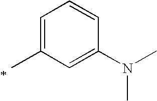 | 3.87 | 357.4 | 99 |
| 112 | 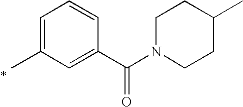 | 4.36 | 439.4 | 91 |
| 113 | 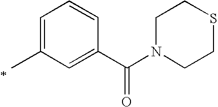 | 7.31* | 443.4 | 97 |
| 114 | 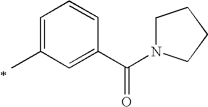 | 3.88 | 411.4 | 99 |
| 115 | 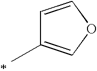 | 3.74 | 304.3 | 99 |
| 116 | 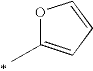 | 3.96 | 304.3 | 99 |

*indicates HPLC Method 3

### Example 5

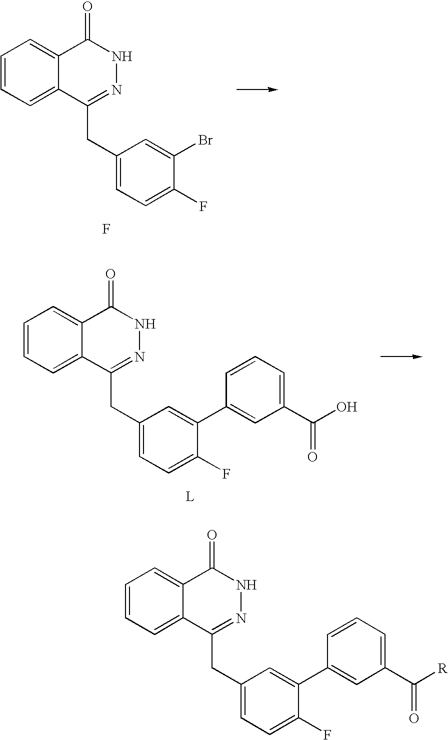

### (a) 2′-Fluoro-5′-(4-oxo-3,4-dihydro-phthalazin-1-ylmethyl)-biphenyl-3-carboxylic acid (L)

To 4-(3-bromo-4-fluoro-benzyl)-2H-phthalazin-1-one (F) (0.400 g, 1.20 mmol) dissolved in acetonitrile (15 mL) and water (5 mL), was added (3-carboxyphenyl)boronic acid (0.198 g, 1.2 mmol), potassium carbonate (496 mg, 3.60 mmol) and tetrakis (triphenylphosphine) palladium (80 mg, 0.069 mmol). The reaction vessel was purged with nitrogen and subjected to sonic agitation for 10 minutes, before being irradiated by microwave, maintaining the reaction at a fixed temperature of 120° C. for 30 minutes. The resultant mixture was then passed through a plug of celite (5 g), eluting with 70:30 acetonitrile:water (15 mL). The filtrate was concentrated in vacuo and crude material taken through to next stage with need for any purification. Single peak in LC-MS, (0.398 g, 95% purity); m/z (LC-MS, ESN), RT=3.25 mins (M−H) 373.2.

### (b) Library Synthesis

To 2′-fluoro-5′-(4-oxo-3,4-dihydro-phthalazin-1-ylmethyl)-biphenyl-3-carboxylic acid (L) (0.02 g, 0.053 mmol) dissolved in DMF (1 mL) was added HBTU (0.04 g, 0.106 mmol), triethylamine (15 μL, 0.106 mmol) and amine (0.058 mmol). The reaction was then stirred for 6 hrs and then submitted for preparative HPLC purification.

The compounds synthesised are set out below:

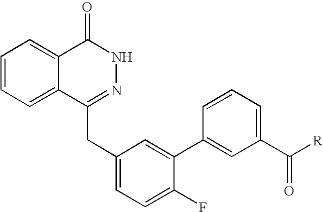

| Com- | | Rt (mins) | M + | Purity |
|---|---|---|---|---|
| pound | R | Method 2 | H | (%) |
| 117 | 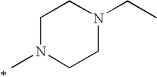 | 3.93 | 471.4 | 97 |
| 118 | 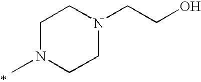 | 3.88 | 487.3 | 99 |
| 119 | 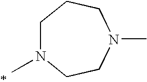 | 7.31* | 471.3 | 93 |
| 120 | 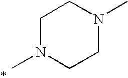 | 3.91 | 457.4 | 98 |
| 121 | 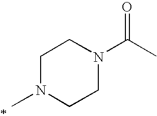 | 7.27* | 501.3 | 94 |
| 122 | 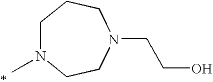 | 9.04* | 501.3 | 97 |
| 123 | 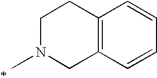 | 5.68 | 490.3 | 99 |
| 124 | 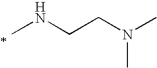 | 3.95 | 445.4 | 99 |
| 125 | 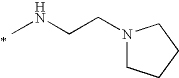 | 4.01 | 471.4 | 99 |
| 126 | 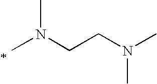 | 7.48* | 459.3 | 94 |

*indicates HPLC Method 3

### Example 6

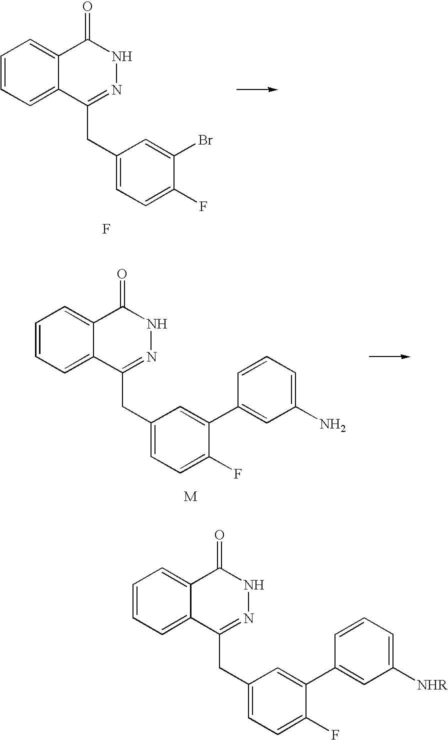

### (a) 4-(3′-Amino-6-fluoro-biphenyl-3-ylmethyl)-2H-phthalazin-1-one (M)

To 4-(3-bromo-4-fluoro-benzyl)-2H-phthalazin-1-one (F) (0.600 g, 1.80 mmol) dissolved in acetonitrile (15 mL) and water (5 mL), was added (3-aminophenyl)boronic acid monohydrate (0.246 g, 1.8 mmol), potassium carbonate (0.745 g, 5.40 mmol) and tetrakis (triphenylphosphine) palladium (69 mg, 0.060 mmol). The reaction vessel was purged with nitrogen and subjected to sonic agitation for 10 minutes, before being irradiated by microwave, maintaining the reaction at a fixed temperature of 140° C. for 20 minutes. The resultant mixture was then passed through a plug of celite (5 g), eluting with ethyl acetate (15 mL). The filtrate was concentrated in vacuo and crude material subjected to flash chromatography using eluent ethyl/acetate hexane 1:1 (rf of 0.17). Single peak in LC-MS, (0.52 g, 95% purity); m/z (LC-MS, ESP), RT=3.97 mins (M+H) 346.2.

### (b) Library Synthesis

To a solution of 4-(3′-amino-6-fluoro-biphenyl-3-ylmethyl)-2H-phthalazin-1-one (M) (0.02 g, 0.060 mmol) in DMF (1 mL) was added HBTU (0.045 g, 0.12 mmol), triethyl amine (0.017 μL, 0.12 mmol) and acid (0.066 mmol). The reaction was stirred at room temperature for 4 hours and then submitted for preparative HPLC purification.

The compounds synthesised are set out below:

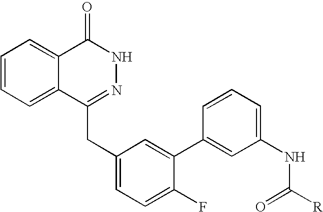

| | | RT (mins) | | Purity |
|---|---|---|---|---|
| Compound | R | Method 2 | M + H | (%) |
| 127 | 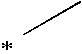 | 4.89 | 388.3 | 98 |
| 128 | 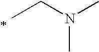 | 7.68* | 431.2 | 99 |
| 129 | 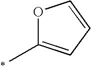 | 5.34 | 440.3 | 99 |
| 130 | 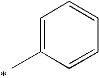 | 5.62 | 450.4 | 94 |
| 131 | 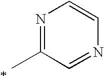 | 5.29 | 452.2 | 96 |
| 132 | 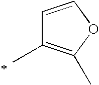 | 5.29 | 454.4 | 92 |
| 133 | 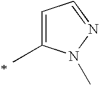 | 11.16* | 454.3 | 92 |
| 134 | 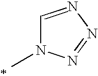 | 4.84⁺ | 456.4 | 88 |
| 135 | 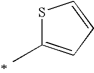 | 5.58⁺ | 456.4 | 95 |
| 136 | 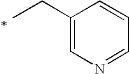 | 4.24⁺ | 465.4 | 98 |
| 137 | 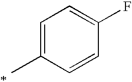 | 5.72⁺ | 468.4 | 92 |
| 138 | 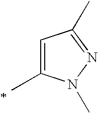 | 5.33⁺ | 468.4 | 91 |
| 139 | 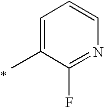 | 5.32⁺ | 469.4 | 90 |
| 140 | 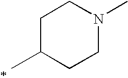 | 7.95* | 471.3 | 95 |
| 141 | 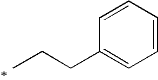 | 5.79⁺ | 478.4 | 99 |
| 142 | 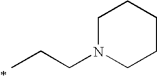 | 4.21⁺ | 485.3 | 98 |
| 143 | 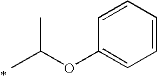 | 5.88⁺ | 494.4 | 99 |
| 144 | 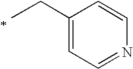 | 4.18⁺ | 465.3 | 99 |
| 145 | 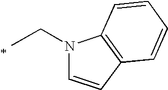 | 5.84 | 503.3 | 99 |
| 146 | 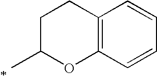 | 6.10 | 506.3 | 98 |

*indicates HPLC Method 3
⁺indicates HPLC Method 4

### (c) Further Library Synthesis

To a solution of 4-(3′-amino-6-fluoro-biphenyl-3-ylmethyl)-2H-phthalazin-1-one (X) (0.02 g, 0.060 mmol) in dry DCM (1 mL) was added pyridine (0.07 μL, 0.080 mmol) and sulfonyl chloride (0.089 mmol). The reaction mixture was stirred for 4 hours and then concentrated in vacuo. The crude solid was then submitted for preparative HPLC purification.

The compounds synthesised are set out below:

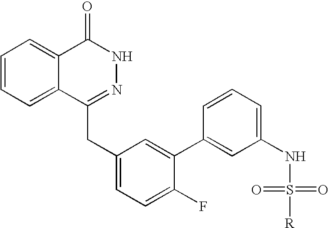

| | | RT (mins) | | Purity |
|---|---|---|---|---|
| Compound | R | Method 3 | M + H | (%) |
| 147 | 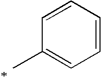 | 12.04 | 486.3 | 99 |
| 148 | 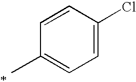 | 13.09 | 520.3 | 100 |
| 149 | 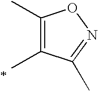 | 11.90 | 505.3 | 100 |
| 150 | 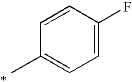 | 12.37 | 504.3 | 99 |
| 151 | 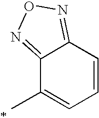 | 12.02 | 528.4 | 91 |
| 152 | 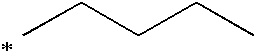 | 12.19 | 466.3 | 100 |
| 153 | 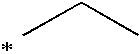 | 10.75 | 438.3 | 99 |
| 154 | 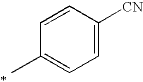 | 12.09 | 511.3 | 99 |
| 155 | 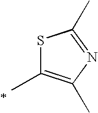 | 11.43 | 521.3 | 100 |
| 156 | 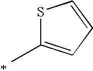 | 11.90 | 492.3 | 100 |
| 157 | 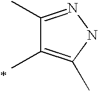 | 10.69 | 518.3 | 98 |
| 158 | 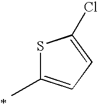 | 13.14 | 526.2 | 99 |
| 159 | 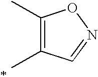 | 11.65 | 491.3 | 100 |

### (d) Further Library Synthesis

To a solution of 4-(3′-amino-6-fluoro-biphenyl-3-ylmethyl)-2H-phthalazin-1-one (M) (0.02 g, 0.060 mmol) in dry THF (1 mL) was added isocyanate (0.080 mmol). The reaction was stirred for 4 hours and then concentrated in vacuo. The crude solid was then submitted for preparative HPLC purification.

The compounds synthesised are set out below:

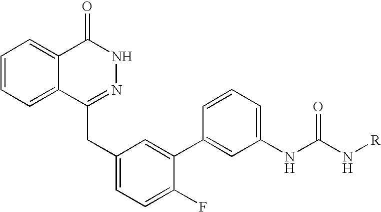

| | | RT (mins) | | Purity |
|---|---|---|---|---|
| Compound | R | Method 2 | M + H | (%) |
| 160 | 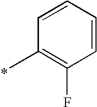 | 5.69 | 483.3 | 96 |
| 161 | 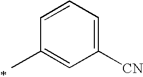 | 5.52 | 490.3 | 98 |
| 162 | 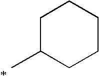 | 5.59 | 471.2 | 96 |
| 163 | 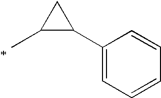 | 5.63 | 505.3 | 98 |

### Example 7

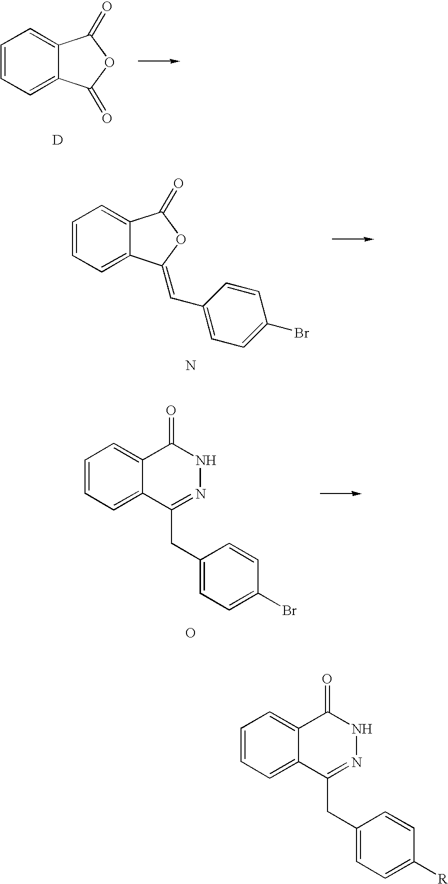

### (a) 3-(4-Bromo-benzylidene)-3H-isobenzofuran-1-one (N)

Phthalic anhydride (D) (11.1 g, 75.3 mmol), 4-bromo-phenylacetic acid (10.1 g, 47 mmol) and sodium acetate (0.170 g, 2.04 mmol) were fused together at 240-245° C. for 25 minutes. The reaction was cool gradually over 30 minutes to 90° C. Ethanol (7 mL) was cautiously added to the reaction mixture and the resultant precipitate filtered and washed with cold ethanol (1×5 mL) before being dried in vacuo. Single peak in LC-MS, (13.5 g, 85% purity) and required no further purification; m/z (LC-MS, ESP), RT=4.72 mins no ionization observed.

### (b) 4-(4-Pyrrol-1-yl-benzyl)-2H-phthalazin-1-one (O)

To 3-(4-bromo-benzylidene)-3H-isobenzofuran-1-one (N) (13.5 g, 38.15 mmol) was suspended in water (260 mL) was added hydrazine hydrate (6.04 mL, 120.0 mmol). The resultant mixture was heated to 100° C. for 24 hours. The reaction was then allowed to cool and the resultant precipitate isolated by filtration yielding the title compound. Single peak in LC-MS, (81 g, 95% purity) and required no further purification; m/z (LC-MS, ESP), RT=3.78 mins with M+H 315 & 317.

### (c) Library Synthesis

To 4-(4-pyrrol-1-yl-benzyl)-2H-phthalazin-1-one (O) (0.030 g, 0.095 mmol) dissolved in acetonitrile (0.75 mL) and water (0.25 mL), was added boronic acid (0.095 mmol), potassium carbonate (21 mg, 0.150 mmol) and tetrakis (triphenylphosphine) palladium (7 mg, 0.006 mmol). The reaction vessel was purged with nitrogen and subjected to sonic agitation for 10 minutes, before being irradiated by microwave, maintaining the reaction at a fixed temperature of 120° C. for 30 minutes. The resultant mixture was then passed through a plug of celite (0.5 g), eluting with ethyl acetate. The filtrate concentrated in vacuo and crude material submitted for preparative HPLC chromatography.

The compounds synthesised are set out below:

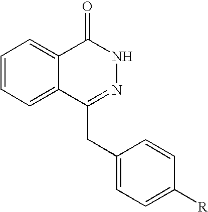

| | | RT (mins) | | Purity |
|---|---|---|---|---|
| Compound | R | Method 3 | M + H | (%) |
| 164 | 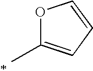 | 11.38 | 303.2 | 82 |
| 165 | 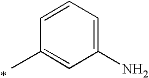 | 7.51 | 328.2 | 94 |
| 166 | 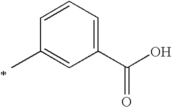 | 9.95 | 357.2 | 86 |
| 167 | 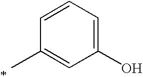 | 10.05 | 329.2 | 90 |
| 168 | 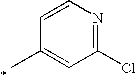 | 10.58 | 348.1 | 93 |
| 169 | 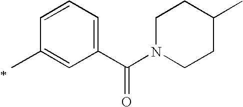 | 12.21 | 438.2 | 99 |
| 170 | 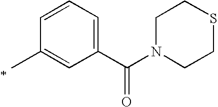 | 10.92 | 442.2 | 93 |
| 171 | 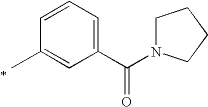 | 10.26 | 410.2 | 99 |
| 172 | 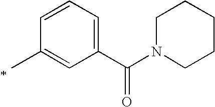 | 11.25 | 424.3 | 98 |
| 173 | 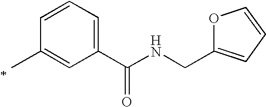 | 11.02 | 436.2 | 95 |
| 174 | 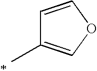 | 11.01 | 303.2 | 94 |

### Example 8

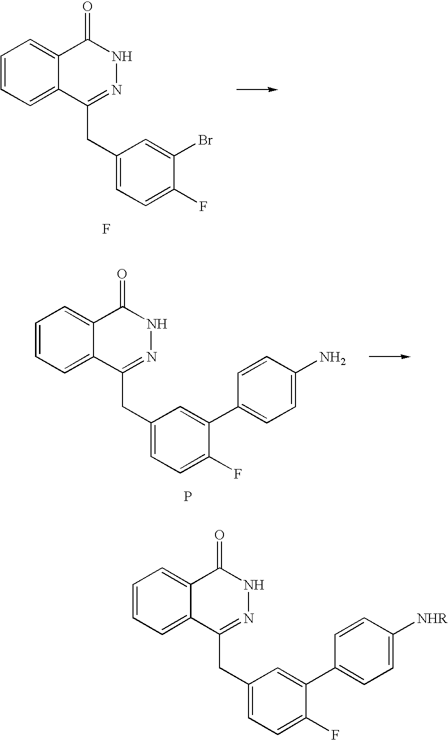

### (a) 4-(4′-Amino-6-fluoro-biphenyl-3-ylmethyl)-2H-phthalazin-1-one (P)

To 4-(3-bromo-4-fluoro-benzyl)-2H-phthalazin-1-one (F) (0.800 g, 2.40 mmol) dissolved in acetonitrile (15 mL) and water (5 mL), was added (4-aminophenyl)boronic acid monohydrate (0.494 g, 1.8 mmol), potassium carbonate (0.992 g, 7.2 mmol) and tetrakis (triphenylphosphine) palladium (150 mg, 0.13 mmol). The reaction vessel was purged with nitrogen and subjected to sonic agitation for 10 minutes, before being irradiated by microwave, maintaining the reaction at a fixed temperature of 140° C. for 35 minutes. The resultant mixture was then passed through a plug of celite (5 g), eluting with acetonitrile (15 mL). The filtrate was concentrated in vacuo and crude material subjected to flash chromatography using eluent ethyl/acetate hexane 1:1 (Rf=0.20). Single peak in LC-MS, (0.80 g, 94% purity); m/z (LC-MS, ESP), RT=3.09 mins (M+H) 346.2.

### (b) Library Synthesis

### Amides

To a solution of 4-(4′-amino-6-fluoro-biphenyl-3-ylmethyl)-2H-phthalazin-1-one (P) (0.02 g, 0.060 mmol) in DMF (1 mL) was added HBTU (0.045 g, 0.12 mmol), triethyl amine (0.017 μL, 0.12 mmol) and acid (0.066 mmol). The reaction was stirred at room temperature for 4 hours and then submitted for preparative HPLC purification.

The compounds synthesised are set out below:

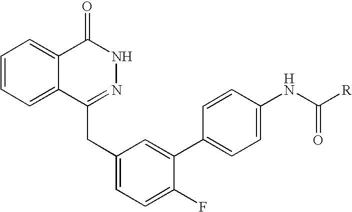

| Com- | | RT (mins) | | Purity |
|---|---|---|---|---|
| pound | R | Method 3 | M + H | (%) |
| 175 | 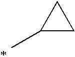 | 10.96 | 414.3 | 92 |
| 176 | 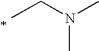 | 7.38 | 431.4 | 92 |
| 177 | 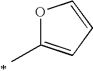 | 11.29 | 440.3 | 95 |
| 178 | 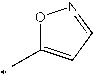 | 10.94 | 441.3 | 99 |
| 179 | 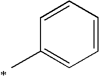 | 12.31 | 450.4 | 80 |
| 180 | 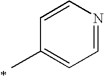 | 9.67 | 451.4 | 100 |
| 181 | 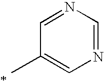 | 11.09 | 452.3 | 99 |
| 182 | 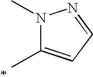 | 11.05 | 454.4 | 99 |
| 183 | 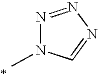 | 9.70 | 456.4 | 100 |
| 184 | 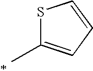 | 12.20 | 456.3 | 97 |
| 185 | 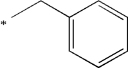 | 12.30 | 464.4 | 100 |
| 186 | 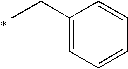 | 9.38 | 465.3 | 93 |
| 187 | 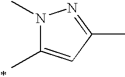 | 12.64 | 468.4 | 81 |
| 188 | 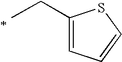 | 12.08 | 470.3 | 100 |
| 189 | 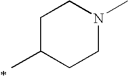 | 7.70 | 471.3 | 93 |
| 190 | 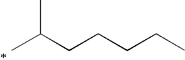 | 14.54 | 472.4 | 99 |
| 191 | 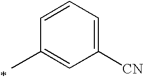 | 12.36 | 475.3 | 100 |
| 192 | 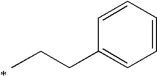 | 12.93 | 478.3 | 100 |
| 193 | 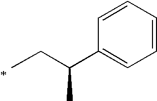 | 13.27 | 492.4 | 99 |
| 194 | 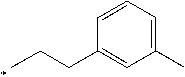 | 12.61 | 492.4 | 97 |
| 195 | 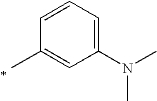 | 12.79 | 493.3 | 99 |
| 196 | 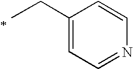 | 7.74 | 465.4 | 100 |

### (c) Library Synthesis

### Urethanes

To a solution of 4-(4′-amino-6-fluoro-biphenyl-3-ylmethyl)-2H-phthalazin-1-one (P) (0.02 g, 0.060 mmol) in dry THF (1 mL) was added isocyanate (0.080 mmol). The reaction was stirred for 4 hours and then concentrated in vacuo. The crude solid was then submitted for preparative HPLC purification.

The compounds synthesised are set out below:

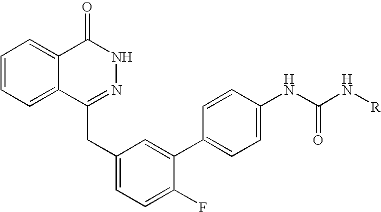

| | | RT (mins) | | Purity |
|---|---|---|---|---|
| Compound | R | Method 2 | M + H | (%) |
| 197 | 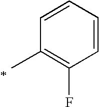 | 5.79 | 483.3 | 98 |
| 198 | 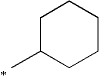 | 5.65 | 471.3 | 99 |
| 199 | 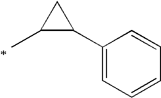 | 5.83 | 505.4 | 92 |
| 200 | 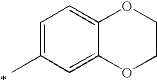 | 5.48 | 523.3 | 97 |
| 201 | 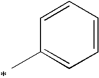 | 5.63 | 465.3 | 99 |

### (c) Library Synthesis

### Sulfonamides

To a solution of 4-(4′-amino-6-fluoro-biphenyl-3-ylmethyl)-2H-phthalazin-1-one (P) (0.02 g, 0.060 mmol) in dry DCM (1 mL) was added pyridine (0.07 μL, 0.075 mmol) and sulfonyl chloride (0.075 mmol). The reaction mixture was stirred for 4 hours and then concentrated in vacuo. The crude solid was then submitted for preparative HPLC purification.

The compounds synthesised are set out below:

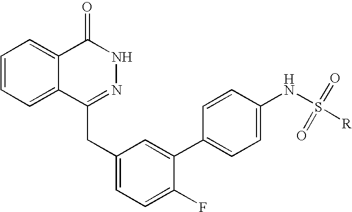

| | | RT (mins) | | Purity |
|---|---|---|---|---|
| Compound | R | Method 2 | M + H | (%) |
| 202 | 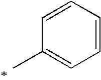 | 5.41 | 486.3 | 99 |
| 203 | 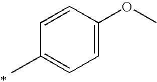 | 5.43 | 516.3 | 99 |
| 204 | 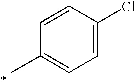 | 5.72 | 520.2 | 99 |
| 205 | 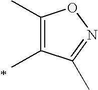 | 5.39 | 505.3 | 99 |
| 206 | 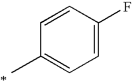 | 5.52 | 504.3 | 99 |
| 207 | 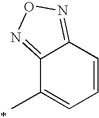 | 5.44 | 528.3 | 99 |
| 208 | 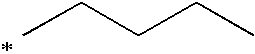 | 5.47 | 466.3 | 99 |
| 209 | 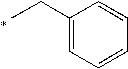 | 5.53 | 500.3 | 99 |
| 210 | 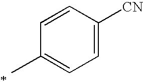 | 5.41 | 511.3 | 99 |
| 211 | 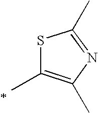 | 5.25 | 521.3 | 96 |
| 212 | 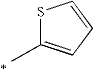 | 5.37 | 492.3 | 99 |
| 213 | 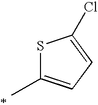 | 29.50* | 526.2 | 95 |
| 214 | 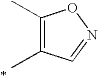 | 5.29 | 491.3 | 99 |

*213 was found to have a shoulder peak with similar retention. To improve the resolution of the chromatography a method was developed, as follows Instrument: Waters ZMD LC-MS system No. LD352 operating in Electrospray ionisation mode.

Mobile Phase A: 0.1% Formic acid in water
Mobile Phase B: 0.1% Formic acid in acetonitrile

### Column: Genesis C18 4 μm 50×4.6 mm Serial no. 1101808

### Gradient:

| Time (mins.) | % B |
|---|---|
| 0 | 5 |
| 7 | 95 |
| 9.5 | 95 |
| 10 | 5 |
| 13 | 5 |

Flow rate: 11.0 ml/min.
PDA Scan range: 210-400 nm.

### (d) Library Synthesis

### Further Sulfonamides

A group of non-commercial sulfonyl chlorides were synthesised following a method reported by T. B. Johnson, J. M. Sprague, J. Am. Chem. Soc., 1936, 58, 1348.

For example (tetrahydro-furan-2-yl)-methanesulfonyl chloride was synthesised from 2-chloromethyl-tetrahydro-furan using the following method:

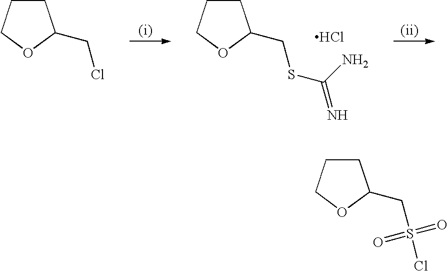

(i) A suspension of 2-chloromethyltetrahydrofuran (5.0 g, 41.47 mmols), thiourea (3.16 g, 41.47 mmols) and potassium iodide (50 mg, 0.30 mmol) in absolute ethanol (30 mL) was heated at 90° C. for 3 hours. The ethanol was removed under reduced pressure to afford a crude isothiourea salt assumed to be quantitative in yield.

(ii) A solution of crude isothiourea salt (41.47 mmol) in water (50 mL) was cooled to 0° C. by an external ice-water bath and chlorine gas bubbled through at such a rate so as to not raise the temperature above 10° C. The treatment with chlorine was continued until the oil layer had settled out and the aqueous layer turned pale green. The oil layer was then extracted from the aqueous layer into diethyl ether (2×50 mL) the resulting in solution was washing with saturated sodium thiosulfate solution (3×10 mL) followed by brine (20 mL). The organic phase was then dried over MgSO₄ and concentrated in vacuo to afford a yellow oil of (1.79 g, 9.694 mmols).

Related sulfonyl chlorides were prepared by an analogous method.

These sulfonyl chlorides were then used in the same way as step (c) above. The compounds synthesised are set out below:

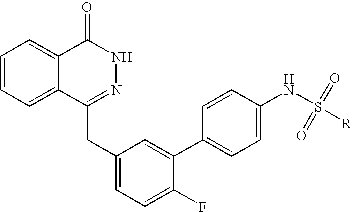

| | | RT (mins) | | Purity |
|---|---|---|---|---|
| Compound | R | Method 2 | M + H | (%) |
| 242 | 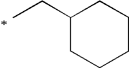 | 5.96 | 506.3 | 99 |
| 243 | 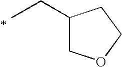 | 4.96 | 494.1 | 98 |
| 244 | 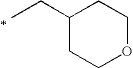 | 10.83* | 508.2 | 89 |
| 245 | 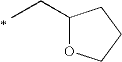 | 10.57* | 458.1 | 97 |
| 246 | 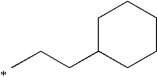 | 6.18 | 520.3 | 98 |
| 247 | 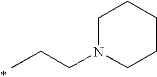 | 8.07* | 485.2 | 92 |
| 248 | 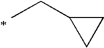 | 5.27 | 464.1 | 97 |

*indicates Method 3

### Example 9

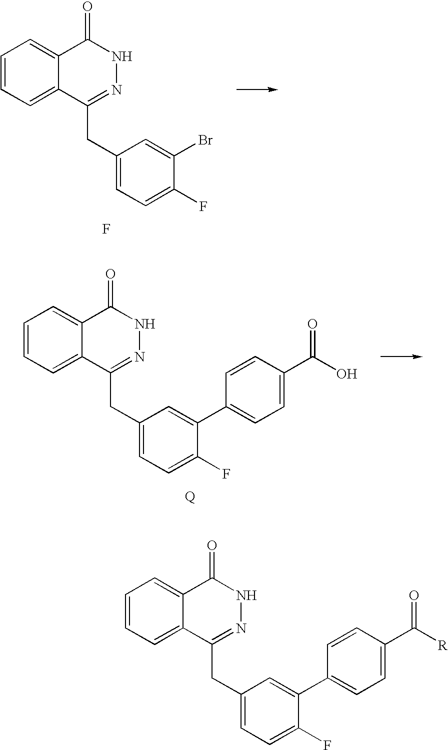

### (a) 2′-Fluoro-5′-(4-oxo-3,4-dihydro-phthalazin-1-ylmethyl)-biphenyl-4-carboxylic acid (Q)

To 4-(3-bromo-4-fluoro-benzyl)-2H-phthalazin-1-one (F) (0.400 g, 1.20 mmol) dissolved in acetonitrile (15 mL) and water (5 mL), was added (4-carboxyphenyl)boronic acid (0.198 g, 1.2 mmol), potassium carbonate (496 mg, 3.60 mmol) and tetrakis (triphenylphosphine) palladium (81 mg, 0.069 mmol). The reaction vessel was purged with nitrogen and subjected to sonic agitation for 10 minutes, before being irradiated by microwave, maintaining the reaction at a fixed temperature of 120° C. for 30 minutes. The resultant mixture was then passed through a plug of celite (5 g), eluting with 70:30 acetonitrile:water (15 mL). The filtrate was concentrated in vacuo and crude material taken through to next stage with need for any purification. Single peak in LC-MS, (0.342 g, 100% purity); m/z (LC-MS, ESN), RT=4.17 mins (M−H) 373.2.

### (b) Library Synthesis

To 2′-fluoro-5′-(4-oxo-3,4-dihydro-phthalazin-1-ylmethyl)-biphenyl-4-carboxylic acid (Q) (0.02 g, 0.053 mmol) dissolved in DMF (1 mL) was added HBTU (0.04 g, 0.106 mmol), triethylamine (15 μL, 0.106 mmol) and amine (0.058 mmol). The reaction was then stirred for 6 hrs and then submitted for preparative HPLC purification.

The compounds synthesised are set out below:

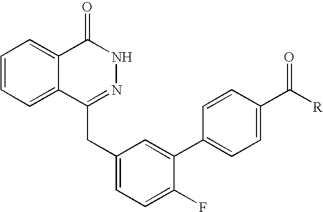

| | | RT | | |
|---|---|---|---|---|
| | | (mins) | | |
| Com- | | Method | | Purity |
| pound | R | 2 | M + H | (%) |
| 215 | 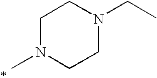 | 4.07 | 471.3 | 95 |
| 216 | 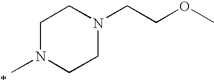 | 4.11 | 501.4 | 99 |
| 217 | 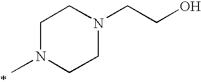 | 4.01 | 487.3 | 99 |
| 218 | 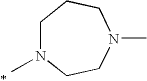 | 4.06 | 471.3 | 99 |
| 219 | 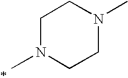 | 4.03 | 457.4 | 99 |
| 220 | 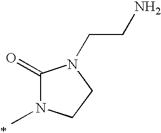 | 4.52 | 486.3 | 99 |
| 221 | 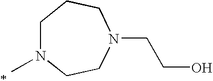 | 7.30* | 501.4 | 94 |
| 222 | 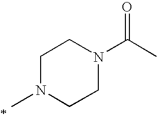 | 4.60 | 485.3 | 99 |
| 223 | 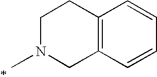 | 12.83* | 490.4 | 98 |

### Example 10

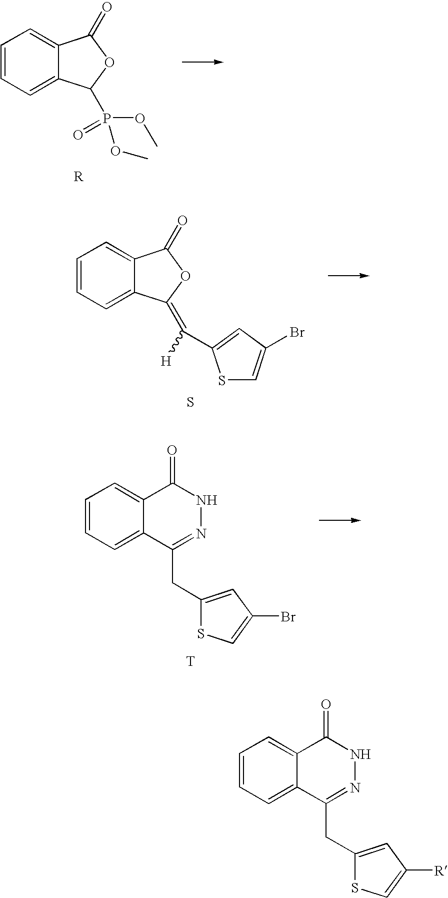

### (a) 3-(4-Bromo-thiophen-2-ylmethylene)-3H-isobenzofuran-1-one (S)

To a solution of (3-oxo-1,3-dihydro-isobenzofuran-1-yl)-phosphonic acid dimethyl ester (R) (6.34 g, 26.17 mmol) in dry THF (65 mL) was added 4-bromo-2-thiophene-carboxaldehyde (5.0 g, 26.17 mmol). The solution was then heated to 55° C. o/n. The reaction was then concentrated in vacuo to afford a crude solid. Main peaks in LC-MS, of both geometric isomers (10 g, 74% purity (combined) and taken through unpurified to the next stage; m/z (LC-MS, ESP), (Isomer A) RT=4.73 mins (M+H) 308, 310; (Isomer B) RT=5.08 mins (M+H) 308, 310.

### (b) 4-(4-Bromo-thiophen-2-ylmethyl)-2H-phthalazin-1-one (T)

To a suspension of 3-(4-bromo-thiophen-2-ylmethylene)-3H-isobenzofuran-1-one (S) (8.0 g, 26.17 mmol) in water (60 mL) was added hydrazine hydrate (1.9 mL, 39.26 mmol) and heated to 90° C. for 18 hours. The reaction cooled to room temperature and resultant precipitate filtered off and washed with water (1×10 mL) followed by diethyl ether (2×15 mL). Single peak in LC-MS, (7.9 g, 95% purity) and taken through unpurified to the next stage; m/z (LC-MS, ESP), RT=3.69 mins (M+H) 321 & 323;

### (c) Library Synthesis

To a suspension of 4-(4-bromo-thiophen-2-ylmethyl)-2H-phthalazin-1-one (T) (0.020 g, 0.060 mmol) in acetonitrile (0.75 mL) and water (0.5 mL) was added boronic acid (0.090 mmol), potassium carbonate (25 mg, 0.180 mmol) and tetrakis (triphenylphosphine) palladium (4 mg, 0.004 mmol). The reaction vessel was purged with nitrogen and subjected to sonic agitation for 10 minutes, before being irradiated by microwave, maintaining the reaction at a fixed temperature of 140° C. for 10 minutes.

The resultant mixture was then passed through a plug of celite (0.5 g), eluting with ethyl acetate. The filtrate concentrated in vacuo and crude material submitted for preparative HPLC chromatography.

The compounds synthesised are set out below:

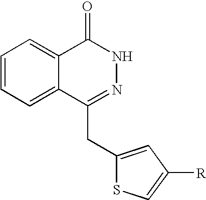

| | | RT (mins) | | Purity |
|---|---|---|---|---|
| Compound | R | Method 3 | M + H | (%) |
| 224 | 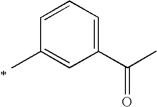 | 10.96 | 361.2 | 92 |
| 225 | 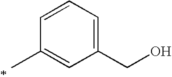 | 9.42 | 649.2 | 94 |
| 226 | 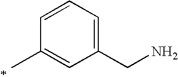 | 6.76 | 648.3 | 95 |
| 227 | 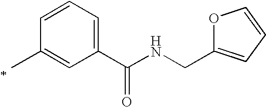 | 10.90 | 442.2 | 88 |
| 228 | 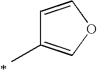 | 10.72 | 309.2 | 97 |

### Example 11

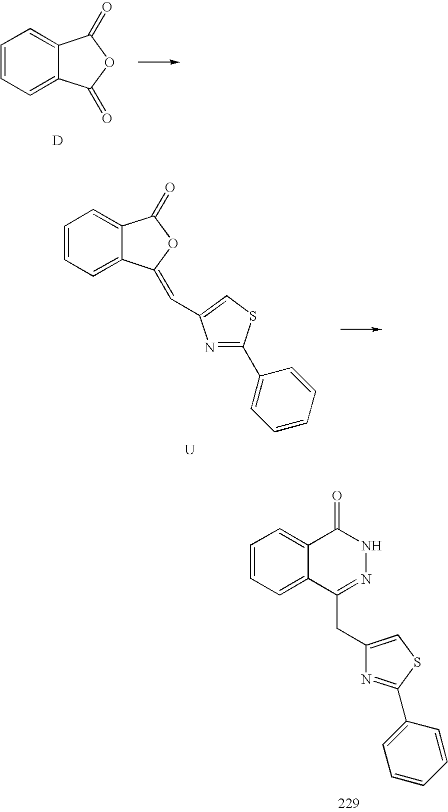

### (a) 3-(2-Phenyl-thiazol-4-ylmethylene)-3H-isobenzofuran-1-one (U)

Phthalic anhydride (D) (0.290 g, 1.96 mol), 2,(2-phenyl-1,3,thizole-4-yl)acetic acid (0.430 g, 1.96 mmol) and sodium acetate were fused together at 240°-245° C. for 20 minutes. The reaction mixture was then cooled over a period of 1 hour to 90° C. Ethanol (8 ml) was added to the mixture and resultant suspension was triturated in the ethanol for 15 mins and then filtered. The solid was taken through crude to the next stage, Main peak in LC-MS, (0.59 g, 60% purity); m/z (LC-MS, ESP), RT=5.35 mins no ionization observed.

### (b) 4-(2-Phenyl-thiazol-4-ylmethyl)-2H-phthalazin-1-one (229)

To a suspension of 3-(2-phenyl-thiazol-4-ylmethylene)-3H-isobenzofuran-1-one (U) (0.59 g, 1.96 mmol) in water (10 ml) was added hydrazine hydrate (0.28 mL, 5.87 mmol).

The reaction was heated at 95° C. for 24 hrs. The reaction mixture was cooled and the precipitate that resulted was isolated by filtration and washed with ice water (3 ml). The solid was then dried in vacuo o/n. Single peak in LC-MS, (0.336 g, 100% purity); m/z (LC-MS, ESP), RT=3.76 mins (M+H) 320;

### Example 12

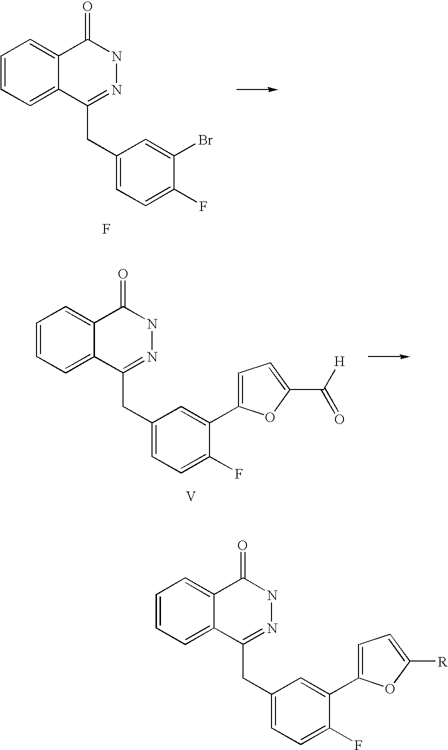

### (a) 5-[2-Fluoro-5-(4-oxo-3,4-dihydro-phthalazin-1-ylmethyl)-phenyl]-furan-2-carbaldehyde (V)

To 4-(3-bromo-4-fluoro-benzyl)-2H-phthalazin-1-one (F) (1.10 g, 3.20 mmol) dissolved in dioxane (17 mL) and DMF (2 mL) was added to (5-formyl-2 furylboronic acid (0.460 g, 3.3 mmol), tripotassium phosphate (1.908 g, 9 mmol) and bis(tri-t-butylphosphine) palladium (228 mg, 0.450 mmol). The reaction vessel was purged with nitrogen and subjected to sonic agitation for 10 minutes, before being irradiated by microwave, maintaining the reaction at a fixed temperature of 120° C. for 40 minutes. The resultant mixture was then passed through a plug of celite (5 g), eluting with ethyl acetate. The filtrate was concentrated in vacuo and crude material taken through to next stage with need for any purification. Single peak in LC-MS, (0.6 g, 94% purity); m/z (LC-MS, ESN), RT=4.10 mins (M+H) 349;

### (b) Library Synthesis

To a solution of 5-[2-fluoro-5-(4-oxo-3,4-dihydro-phthalazin-1-ylmethyl)-phenyl]-furan-2-carbaldehyde (V) (0.02 g, 0.057 mmol) in THF (1 mL) and DCM (1 mL) was added amine (0.052 mmol), acetic acid (0.01 μL, 0.002 mmol) and sodium borohydride (2 mg, 0.063 mmol). The mixture was stirred at room temperature overnight and then filtered through celite (0.5 g) eluting with DCM (3 mL). The filtrate was then concentrated in vacuo and submitted for preparative HPLC purification.

The compounds synthesised are set out below:

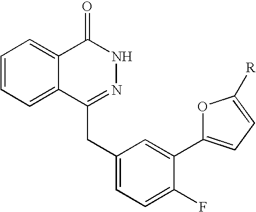

| | | RT | | |
|---|---|---|---|---|
| | | (mins) | | |
| Com- | | Method | | Purity |
| pound | R | 2 | M + H | (%) |
| 230 | 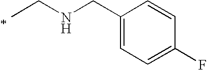 | 4.23 | 458.3 | 99 |
| 231 | 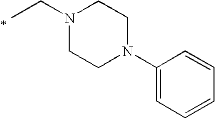 | 4.37 | 495.4 | 99 |
| 232 | 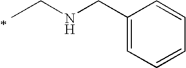 | 4.19 | 440.3 | 99 |
| 233 | 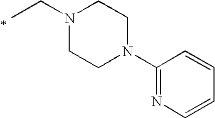 | 3.95 | 496.4 | 99 |
| 234 | 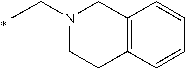 | 4.25 | 466.4 | 99 |
| 235 | 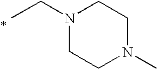 | 3.94 | 433.3 | 99 |
| 236 | 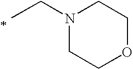 | 3.89 | 420.3 | 99 |
| 237 | 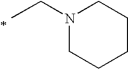 | 4.03 | 418.3 | 98 |
| 238 | 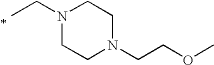 | 4.01 | 477.3 | 98 |
| 239 | 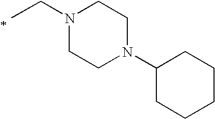 | 4.24 | 501.4 | 99 |
| 240 | 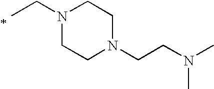 | 3.55 | 490.4 | 99 |
| 241 | 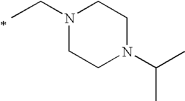 | 4.00 | 461.3 | 99 |

### Example 13

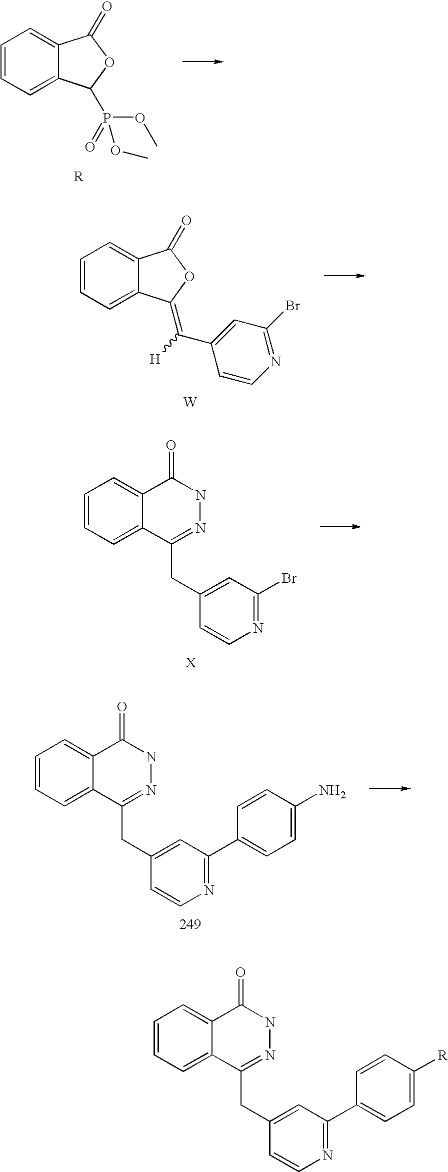

### (a) 3-(2-Bromo-pyridin-4-ylmethylene)-3H-isobenzofuran-1-one (W)

To a solution of (3-Oxo-1,3-dihydro-isobenzofuran-1-yl)-phosphonic acid dimethyl ester (R) (3.09, 12.76 mmol) in dry THF (40 mL) was added 2-bromo-pyridine-4-carbaldehyde (2.37 g, 12.76 mmol) and triethyl amine (1.76 ml, 12.76 mL). The solution was stirred at room temperature for 24 hours. The reaction mixture was cooled 5° C. and diluted with ice water (50 mL) and stirred for 0.5 hour. The resultant solid was filtered and washed with hexane (2×20 mL) and dried in vacuum oven to afford a white powder. Two isomeric cpds in LC-MS, (combined 3.83 g, 100% purity) and taken through without need for purification to the next stage; m/z (LC-MS, ESP), RT=3.95 mins (M+H) 302 & RT=4.01 mins (M+H) 302.

### (b) 4-(2-Bromo-pyridin-4-ylmethyl)-2H-phthalazin-1-one (X)

To a suspension of 3-(2-bromo-pyridin-4-ylmethylene)-3H-isobenzofuran-1-one (W) (3.83 g, 12.7 mmol) in water (10 mL) was added hydrazine hydrate (3 mL, 60.0 mmol) and heated to 90° C. for 3 hours. The reaction mixture was then cooled to 5° C. and resultant precipitate filtered and washed with water (3×10 mL) followed by diethyl ether (2×15 mL). The solid was then dried in vacuum oven overnight. Single peak in LC-MS (3.3 g, 98% purity) and taken through unpurified to the next stage; m/z (LC-MS, ESP), RT=3.06 mins (M+H) 316.

### (c) 4-[2-(4-Amino-phenyl)-pyridin-4-ylmethyl]-2H-phthalazin-1-one (249)

To a suspension of 4-(2-bromo-pyridin-4-ylmethyl)-2H-phthalazin-1-one (1.0 g, 3.17 mmol) in acetonitrile (8 mL) and water (4 mL) was added 4-(aminophenyl) boronic acid (646 mg, 4.75 mmol), potassium carbonate (1.38 g, 10.0 mmol) and tetrakis (triphenylphosphine) palladium (4 mg, 0.004 mmol). The reaction vessel was purged with nitrogen and subjected to sonic agitation for 10 minutes, before being irradiated by microwave, maintaining the reaction at a fixed temperature of 140° C. for 10 minutes. The resultant mixture was then passed through a plug of silica gel (1.0 g), eluting with ethyl acetate (25 mL). The filtrate concentrated in vacuo to afford a yellow solid. Single peak in LC-MS, (0.704 g, 99% purity) and taken through unpurified to the next stage; m/z (LC-MS, ESP), RT=2.31 mins (M+H) 329.

### (d) Library Synthesis

To a solution of 4-[2-(4-amino-phenyl)-pyridin-4-ylmethyl]-2H-phthalazin-1-one (249) (0.03 g, 0.091 mmol) in anhydrous DMF (1 mL) was added sulfonyl chlorides (0.150 mmol), followed by triethylamine (34 μL, 0.250 mmol). The mixture was stirred at room temperature overnight and then submitted for preparative HPLC purification.

The compounds synthesised are set out below:

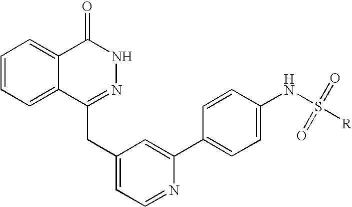

| | | RT (mins) | | Purity |
|---|---|---|---|---|
| Compound | R | Method 2 | M + H | (%) |
| 250 | 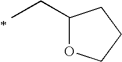 | 3.99 | 477.3 | 97 |
| 251 | 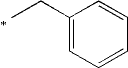 | 4.41 | 483.3 | 85 |
| 252 | 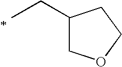 | 3.87 | 477.2 | 96 |

### (e) Further Library Synthesis

To a solution of 4-[2-(4-amino-phenyl)-pyridin-4-ylmethyl]-2H-phthalazin-1-one (249) (0.03 g, 0.091 mmol) in anhydrous DMF (1 mL) was added acids (0.090 mmol), followed by triethylamine (34 μL, 0.250 mmol) and HBTU (34 mg, 0.91 mmol). The mixture was stirred at room temperature overnight and then submitted for preparative HPLC purification.

The compounds synthesised are set out below:

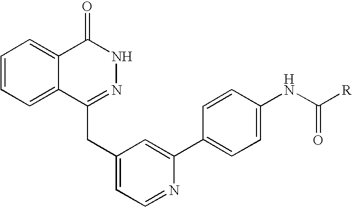

| | | RT (mins) | | Purity |
|---|---|---|---|---|
| Compound | R | Method 2 | M + H | (%) |
| 253 | 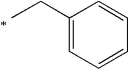 | 4.28 | 447.2 | 96 |
| 254 | 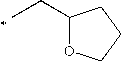 | 3.79 | 441.2 | 99 |

### Example 14

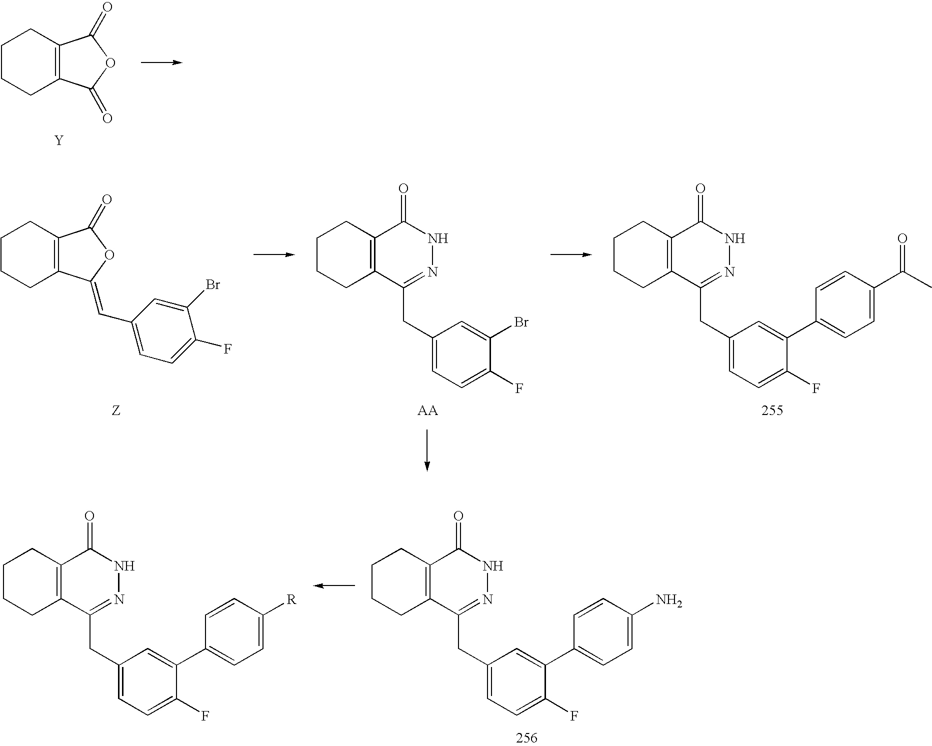

### (a) 3-(3-Bromo-4-fluoro-benzylidene)-4,5,6,7-tetrahydro-3H-isobenzofuran-1-one (Z)

4,5,6,7-Tetrahydro-isobenzofuran-1,3-dione (Y) (16.7 g, 109.7 mmol) and 3-bromo-4-fluorophenylacetic acid (15.0 g, 64.37 mmol) were fused in the presence of sodium acetate (0.259 g, 3.160 mmol) at 210° C. using a ‘Wood's Alloy’ bath for 4.5 hours. The reaction mixture was then poured into a crucible and cooled to give a crystalline solid. The solid was ground with a mortar and pestle and triturated with ethanol (20 mL) for 30 minutes. The resultant suspension was then filtered and washed with further ethanol (1×5 mL). The solid was then dried to afford the desired product as a mixture of geometric isomers. Main peak in LC-MS, (20.78 g, 94% purity) and required no further purification; m/z (LC-MS, ESP), RT=4.74 mins (no ionization observed).

### (b) 4-(3-Bromo-4-fluoro-benzyl)-5,6,7,8-tetrahydro-2H-phthalazin-1-one (AA)

To 3-(3-bromo-4-fluoro-benzylidene)-4,5,6,7-tetrahydro-3H-isobenzofuran-1-one (cis I trans mixture) (20.78 g, 64.3 mmol) suspended in water (150 mL) was added hydrazine hydrate (12.5 ml, 257.2 mmol). The reaction was heated to 85° C. for 18 hours and then cooled to room temperature. A beige suspension was isolated by filtration and washed with water (1×50 mL), hexane (1×50 mL), and ether (1×25 mL) before being dried overnight in a vacuum oven. Main peak in LC-MS, (19.1 g, 91% purity) and required no further purification; m/z (LC-MS, ESP), RT=3.92 mins (M+H 337 & 339).

### (c) 4-(4′-Acetyl-6-fluoro-biphenyl-3-ylmethyl)-5,6,7,8-tetrahydro-2H-phthalazin-1-one (255)

To a suspension of 4-(3-bromo-4-fluoro-benzyl)-5,6,7,8-tetrahydro-2H-phthalazin-1-one (AA) (50.0 mg, 0.15 mmol) in acetonitrile (1.4 mL) and water (0.35 mL) was added 4-acetylphenylboronic acid (49 mg, 0.222 mmol) and potassium carbonate (61 mg, 0.444 mmol) and tetrakis (triphenylphosphine) palladium (4 mg, 0.004 mmol). The reaction vessel was purged with nitrogen and subjected to sonic agitation for 10 minutes, before being irradiated by microwave, maintaining the reaction at a fixed temperature of 140° C. for 10 minutes. The resultant mixture was then passed through a plug of silica gel (0.3 g), eluting with ethyl acetate (25 mL). The filtrate concentrated in vacuo to afford crude yellow oil which was then submitted for preparative HPLC purification. RT (method 2)=5.3 mins; M+H=376.43; Purity 99%.

### (d) 4-(4′-Amino-6-fluoro-biphenyl-3-ylmethyl)-5,6,7,8-tetrahydro-2H-phthalazin-1-one (256)

To a suspension of 4-(3-bromo-4-fluoro-benzyl)-5,6,7,8-tetrahydro-2H-phthalazin-1-one (AA) (1.0 g, 2.99 mmol) in acetonitrile (8 mL) and water (4 mL) was added 4-(aminophenyl) boronic acid (0.69 g, 5.04 mmol), potassium carbonate (1.23 g, 8.9 mmol) and tetrakis (triphenylphosphine) palladium (4 mg, 0.004 mmol). The reaction vessel was purged with nitrogen and subjected to sonic agitation for 10 minutes, before being irradiated by microwave, maintaining the reaction at a fixed temperature of 140° C. for 10 minutes. The resultant mixture was then passed through a plug of silica gel (1.0 g), eluting with ethyl acetate (25 mL). The filtrate concentrated in vacuo to afford a yellow solid. Single peak in LC-MS, (920 mg, 95% purity) and taken through unpurified to the next stage; m/z (LC-MS, ESP), RT=3.98 mins (M+H) 349.

### (e) Library Synthesis

To a solution of 4-(4′-amino-6-fluoro-biphenyl-3-ylmethyl)-5,6,7,8-tetrahydro-2H-phthalazin-1-one (256) (0.025 g, 0.072 mmol) in anhydrous THF (1 mL) was added the appropriate sulfonyl chloride (0.086 mmol), followed by triethylamine (34 μL, 0.250 mmol). The mixture was stirred at room temperature overnight and then submitted for preparative HPLC purification.

The compounds synthesised are set out below:

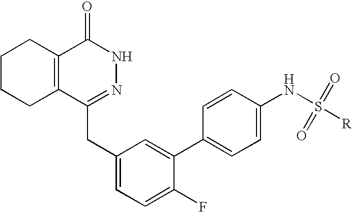

| | | RT (mins) | | Purity |
|---|---|---|---|---|
| Compound | R | Method 2 | M + H | (%) |
| 257 | 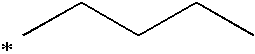 | 12.34* | 470.2 | 98 |
| 258 | 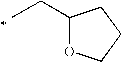 | 11.25 | 498.2 | 91 |
| 259 | 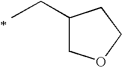 | 10.61* | 498.2 | 90 |
| 260 | 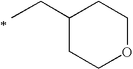 | 10.9* | 512.2 | 92 |
| 261 | 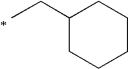 | 6.06 | 510.3 | 97 |
| 262 | 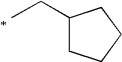 | 5.80 | 496.3 | 96 |
| 263 | 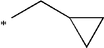 | 5.31 | 468.1 | 94 |

*indicates Method 3

### (e) Further Library Synthesis

To a solution of 4-(4′-amino-6-fluoro-biphenyl-3-ylmethyl)-5,6,7,8-tetrahydro-2H-phthalazin-1-one (256) (0.03 g, 0.086 mmol) in anhydrous DMF (1 mL) was added acids (0.090 mmol), followed by DIPEA (34 μL, 0.250 mmol) and HBTU (34 mg, 0.91 mmol). The mixture was stirred at room temperature overnight and then submitted for preparative HPLC purification.

The compounds synthesised are set out below:

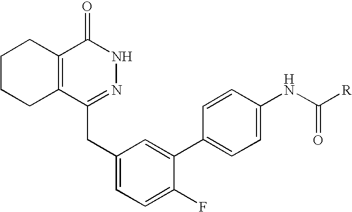

| | | RT (mins) | | Purity |
|---|---|---|---|---|
| Compound | R | Method 3 | M + H | (%) |
| 264 | 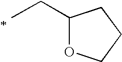 | 4.12 | 489.3 | 94 |
| 265 | 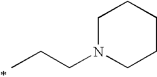 | 12.4* | 468.2 | 88 |
| 266 | 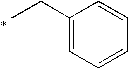 | 12.4* | 468.2 | 88 |
| 267 | 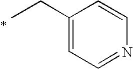 | 4.08 | 469.2 | 99 |

*indicates Method 3

### Example 15

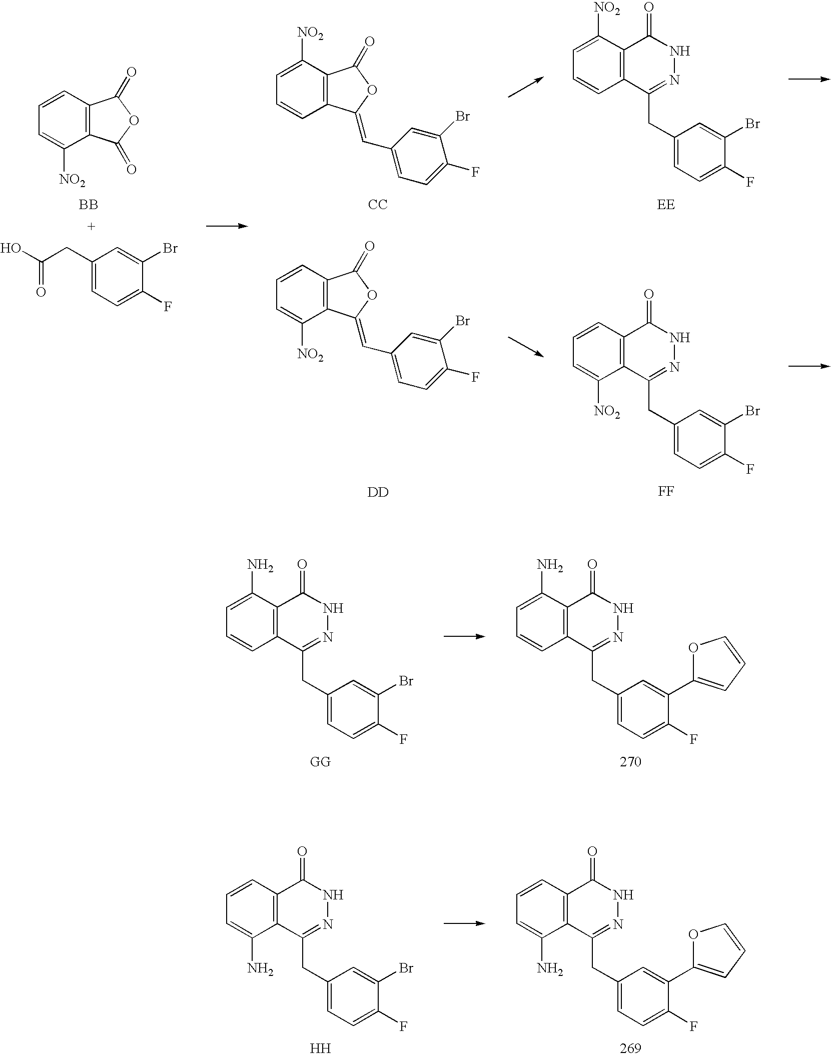

### (a) 3-(3-Bromo-4-fluoro-benzylidene)-7-nitro-3H-isobenzofuran-1-one (CC) & 3-(3-Bromo-4-fluoro-benzylidene)-4-nitro-3H-isobenzofuran-1-one (DD)

4-Nitro-isobenzofuran-1,3-dione (BB) (1.66 g, 8.51 mmol) and 3-bromo-4-fluorophenylacetic acid (1.98 g, 8.51 mmol) were fused in the presence of sodium acetate (0.60 g, 0.40 mmol) at 210° C. using a ‘Wood's Alloy’ bath for 1 hour. The reaction mixture was then poured into a crucible and cooled to give a crystalline solid. The solid was ground with a mortar and pestle and triturated with ethanol (50 mL) for 30 minutes. The resultant suspension was then filtered and washed with further ethanol (2×8 mL). The solid was then dried to afford a mixture of crude geometric & region isomers. The material was subjected to flash chromatography, eluting with hexane/ethyl acetate, 8:3 to afford 2 main isomers later assigned through NOE experiments:

3-(3-Bromo-4-fluoro-benzylidene)-7-nitro-3H-isobenzofuran-1-one (CC): Isolated as a single peak (R_{f} 0.39 in hexane/ethyl acetate, 8:3), LC-MS (107 mg, 95% purity); m/z (LC-MS, ESP), RT=4.58 mins (M+H) (no ionization observed).

3-(3-Bromo-4-fluoro-benzylidene)-4-nitro-3H-isobenzofuran-1-one (DD):

Isolated as a single peak (R_{f} 0.24 in hexane/ethyl acetate, 8:3), LC-MS (50 mg, 95% purity); m/z (LC-MS, ESP), RT=4.43 mins (M+H) (no ionization observed).

### (b) 4-(3-Bromo-4-fluoro-benzyl)-8-nitro-2H-phthalazin-1-one (EE)

To a suspension of 3-(3-bromo-4-fluoro-benzylidene)-7-nitro-3H-isobenzofuran-1-one (CC) (107 mg, 0.29 mmol) in water (4 mL) was added hydrazine hydrate (80 μL, 0.40 mmol). The reaction was heated to 85° C. for 5 hours and then cooled to 5° C. A beige suspension was isolated by filtration and washed with water (2×1 mL), before being dried overnight in a vacuum oven. Main peak in LC-MS, (52 mg, 91% purity) and required no further purification; m/z (LC-MS, ESP), RT=3.81 mins (M+H 380 & 382).

### (c) 8-Amino-4-(3-bromo-4-fluoro-benzyl)-2H-phthalazin-1-one (GG)

To a suspension of 4-(3-bromo-4-fluoro-benzyl)-8-nitro-2H-phthalazin-1-one (EE) (98 mg, 0.260 mmol) in ethanol (1 mL), water (1 mL) was added ammonium chloride 32 mg, 0.60 mmol) followed by iron powder (58 mg, 1.40 mmol). The reaction was then heated to 80° C. for 24 hours and then cooled to room temperature. The reaction mixture was then filtered through a short plug of celite and washed through with methanol (2 column volumes). The crude filtrate was concentrated and to a residue and then subjected to flash chromatography eluent hexane/ethyl acetate 3:2 (R_{f} 0.24). Single peak in LC-MS, (19 mg, 95% purity); m/z (LC-MS, ESP), RT=3.39 mins (M+H) 348 & 350.

### (d) 8-Amino-4-(4-fluoro-3-furan-2-yl-benzyl)-2H-phthalazin-1-one (270)

To a suspension of 8-amino-4-(3-bromo-4-fluoro-benzyl)-2H-phthalazin-1-one (GG) (12 mg, 0.034 mmol) in acetonitrile (0.5 mL) and water (0.1 mL) was added 2-furan boronic acid (5 mg, 0.045 mmol), potassium carbonate (7 mg, 0.051 mmol) and tetrakis (triphenylphosphine) palladium (2 mg, 0.002 mmol). The reaction vessel was purged with nitrogen and subjected to sonic agitation for 10 minutes, before being irradiated by microwave, maintaining the reaction at a fixed temperature of 140° C. for 10 minutes. The resultant mixture was then passed through a plug of silica gel (0.1 g), eluting with ethyl acetate. The filtrate concentrated and the crude reaction mixture was then subjected to preparative HPLC chromatography.

### (e) 4-(3-Bromo-4-fluoro-benzyl)-5-methyl-2H-phthalazin-1-one (FF)

To a suspension of 3-(3-bromo-4-fluoro-benzylidene)-4-nitro-3H-isobenzofuran-1-one (DD) (58 mg, 0.16 mmol) in water (2 mL) was added hydrazine hydrate (80 μL, 0.40 mmol). The reaction was heated to 85° C. for 5 hours and then cooled to 5° C. A beige suspension was isolated by filtration and washed with water (2×1 mL), before being dried overnight in a vacuum oven. Main peak in LC-MS, (60 mg, 89% purity) and required no further purification; m/z (LC-MS, ESP), RT=3.85 mins (M+H 380 & 382).

### (f) 5-Amino-4-(3-bromo-4-fluoro-benzyl)-2H-phthalazin-1-one (HH)

To a suspension of 4-(3-bromo-4-fluoro-benzyl)-5-methyl-2H-phthalazin-1-one (FF) (60 mg, 0.143 mmol) in ethanol (1 mL) and water (1 mL) was added ammonium chloride (10 mg, 0.187 mmol) followed by Iron powder (16 mg, 0.286 mmol). The reaction was then heated to 80° C. for 18 hours and then cooled to room temperature. The reaction mixture was then filtered through a short plug of celite and washed through with methanol (2 column volumes). The crude filtrate was concentrated and to a residue and then subjected to flash chromatography eluent hexane/ethyl acetate 2:1 (R_{f} 0.34). Single peak in LC-MS, (49 mg, 99% purity); m/z (LC-MS, ESP), RT=3.43 mins (M+H) 348 & 350. ¹H NMR (300 MHz, DMSO-D₆), δ ppm 12.14 (s, 1H), 7.51-7.30 (m, 3H), 7.25-6.98 (m, 3H), 5.61 (s, 2H), 4.35 (s, 2H).

### (g) 5-Amino-4-(4-fluoro-3-furan-2-yl-benzyl)-2H-phthalazin-1-one (269)

To a suspension of 5-amino-4-(3-bromo-4-fluoro-benzyl)-2H-phthalazin-1-one (HH) (12 mg, 0.034 mmol) in acetonitrile (0.5 mL) and water (0.1 mL) was added 2-furan boronic acid (5 mg, 0.045 mmol), potassium carbonate (7 mg, 0.051 mmol) and tetrakis (triphenylphosphine) palladium (2 mg, 0.002 mmol). The reaction vessel was purged with nitrogen and subjected to sonic agitation for 10 minutes, before being irradiated by microwave, maintaining the reaction at a fixed temperature of 140° C. for 10 minutes. The resultant mixture was then passed through a plug of silica gel (0.1 g), eluting with ethyl acetate (10 mL). The filtrate concentrated and the crude reaction mixture was then subjected to preparative HPLC chromatography.

| | | RT (mins) | | Purity |
|---|---|---|---|---|
| Compound | | Method 2 | M + H | (%) |
| 269 | 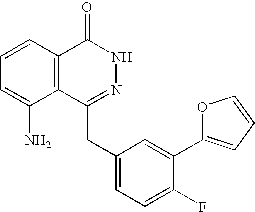 | 10.60* | 336.0 | 82 |
| 270 | 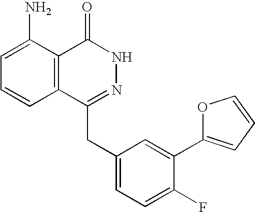 | 10.39* | 336.1 | 81 |

### Example 16

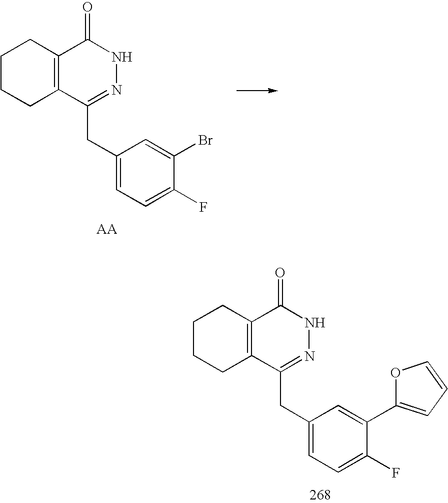

### 4-(4-Fluoro-3-furan-2-yl-benzyl)-5,6,7,8-tetrahydro-2H-phthalazin-1-one

To a suspension of 4-(3-bromo-4-fluoro-benzyl)-5,6,7,8-tetrahydro-2H-phthalazin-1-one (AA) (20 mg, 0.059 mmol) in acetonitrile (1.5 mL) and water (0.5 mL) was added 2-furnaboronic acid (10 mg, 0.089 mmol) and potassium carbonate (24 mg, 0.177 mmol) and tetrakis (triphenylphosphine) palladium (4 mg, 0.004 mmol). The reaction vessel was purged with nitrogen and subjected to sonic agitation for 10 minutes, before being irradiated by microwave, maintaining the reaction at a fixed temperature of 140° C. for 10 minutes. The resultant mixture was then passed through a celite (0.3 g), eluting with ethyl acetate (25 mL). The filtrate concentrated in vacuo to afford crude oil which was then submitted for preparative HPLC purification.

| | RT (mins) | | Purity |
|---|---|---|---|
| Compound | Method 2 | M + H | (%) |
| 268 | 5.49 | 325.3 | 99 |

### Example 17

In order to assess the inhibitory action of the compounds, the following assay was used to determine IC₅₀ values (Dillon, et al., JBS., 8(3), 347-352 (2003)).

Mammalian PARP, isolated from Hela cell nuclear extract, was incubated with Z-buffer (25 mM Hepes (Sigma); 12.5 mM MgCl₂ (Sigma); 50 mM KCl (Sigma); 1 mM DTT (Sigma); 10% Glycerol (Sigma) 0.001% NP-40 (Sigma); pH 7.4) in 96 well FlashPlates (TRADE MARK) (NEN, UK) and varying concentrations of said inhibitors added. All compounds were diluted in DMSO and gave final assay concentrations of between 10 and 0.01 μM, with the DMSO being at a final concentration of 1% per well. The total assay volume per well was 40 μL.

After 10 minutes incubation at 30° C. the reactions were initiated by the addition of a 10 μl reaction mixture, containing NAD (5 μM), ³H-NAD and 30mer double stranded DNA-oligos. Designated positive and negative reaction wells were done in combination with compound wells (unknowns) in order to calculate % enzyme activities. The plates were then shaken for 2 minutes and incubated at 30° C. for 45 minutes.

Following the incubation, the reactions were quenched by the addition of 50 μl 30% acetic acid to each well. The plates were then shaken for 1 hour at room temperature.

The plates were transferred to a TopCount NXT (TRADE MARK) (Packard, UK) for scintillation counting. Values recorded are counts per minute (cpm) following a 30 second counting of each well.

The % enzyme activity for each compound is then calculated using the following equation:

$$\% Inhibition = 100 - ( 100 \times \frac{( cpm of unkowns - mean negative cpm )}{( mean positive cpm - mean negative cpm )} )$$

IC₅₀ values (the concentration at which 50% of the enzyme activity is inhibited) were calculated, which are determined over a range of different concentrations, normally from 10 μM down to 0.001 μM. Such IC₅₀ values are used as comparative values to identify increased compound potencies.

The following compounds displayed an IC₅₀ of less than 1 μM: 9, 10, 17, 21, 23-25, 28, 29, 34, 39, 40, 42, 43, 49, 51-56, 59, 61, 64-66, 68-72, 74, 75, 78, 79, 83, 84, 87-91, 96, 100, 103, 105-107, 109, 110, 115, 116, 118, 119, 121-164, 167, 168, 173-215, 217, 220, 222-241.

The mean IC₅₀ results for compounds of the invention are listed below:

| Compound | IC₅₀ (μM) |
|---|---|
| 9 | 0.914 |
| 10 | 0.261 |
| 17 | 0.196 |
| 21 | 0.196 |
| 23 | 0.018 |
| 24 | 0.105 |
| 25 | 0.082 |
| 28 | 0.039 |
| 29 | 0.027 |
| 34 | 0.284 |
| 39 | 0.201 |
| 40 | 0.297 |
| 42 | 0.060 |
| 43 | 0.065 |
| 49 | 0.041 |
| 51 | 0.029 |
| 52 | 0.075 |
| 53 | 0.014 |
| 54 | 0.089 |
| 55 | 0.279 |
| 56 | 0.218 |
| 59 | 0.085 |
| 61 | 0.300 |
| 64 | 0.114 |
| 65 | 0.904 |
| 66 | 0.038 |
| 68 | 0.489 |
| 69 | 0.720 |
| 70 | 0.175 |
| 71 | 0.064 |
| 72 | 0.062 |
| 74 | 0.287 |
| 75 | 0.412 |
| 78 | 0.511 |
| 79 | 0.149 |
| 83 | 0.316 |
| 84 | 0.585 |
| 87 | 0.275 |
| 88 | 0.067 |
| 89 | 0.338 |
| 90 | 0.389 |
| 91 | 0.061 |
| 92 | 1.297 |
| 93 | 1.126 |
| 96 | 0.264 |
| 100 | 0.268 |
| 103 | 0.403 |
| 105 | 0.452 |
| 106 | 0.751 |
| 107 | 0.198 |
| 109 | 0.174 |
| 110 | 0.481 |
| 112 | 1.201 |
| 113 | 1.258 |
| 114 | 1.331 |
| 115 | 0.404 |
| 116 | 0.083 |
| 117 | 1.102 |
| 118 | 0.912 |
| 119 | 0.547 |
| 120 | 1.019 |
| 121 | 0.865 |
| 122 | 0.816 |
| 123 | 0.125 |
| 124 | 0.353 |
| 125 | 0.258 |
| 126 | 0.805 |
| 127 | 0.009 |
| 128 | 0.079 |
| 129 | 0.026 |
| 130 | 0.033 |
| 131 | 0.104 |
| 132 | 0.021 |
| 133 | 0.021 |
| 134 | 0.056 |
| 135 | 0.029 |
| 136 | 0.007 |
| 137 | 0.042 |
| 138 | 0.031 |
| 139 | 0.039 |
| 140 | 0.065 |
| 141 | 0.029 |
| 142 | 0.097 |
| 143 | 0.117 |
| 144 | 0.009 |
| 145 | 0.031 |
| 146 | 0.081 |
| 147 | 0.053 |
| 148 | 0.076 |
| 149 | 0.027 |
| 150 | 0.071 |
| 151 | 0.036 |
| 152 | 0.019 |
| 153 | 0.014 |
| 154 | 0.022 |
| 155 | 0.038 |
| 156 | 0.041 |
| 157 | 0.014 |
| 158 | 0.123 |
| 159 | 0.035 |
| 160 | 0.009 |
| 161 | 0.023 |
| 162 | 0.088 |
| 163 | 0.102 |
| 164 | 0.393 |
| 165 | 1.517 |
| 166 | 1.180 |
| 167 | 0.624 |
| 168 | 0.379 |
| 170 | 1.892 |
| 171 | 1.990 |
| 173 | 0.788 |
| 174 | 0.309 |
| 175 | 0.057 |
| 176 | 0.736 |
| 177 | 0.162 |
| 178 | 0.196 |
| 179 | 0.171 |
| 180 | 0.088 |
| 181 | 0.078 |
| 182 | 0.218 |
| 183 | 0.236 |
| 184 | 0.155 |
| 185 | 0.054 |
| 186 | 0.086 |
| 187 | 0.285 |
| 188 | 0.031 |
| 189 | 0.554 |
| 190 | 0.277 |
| 191 | 0.211 |
| 192 | 0.085 |
| 193 | 0.237 |
| 194 | 0.190 |
| 195 | 0.169 |
| 196 | 0.119 |
| 197 | 0.120 |
| 198 | 0.036 |
| 199 | 0.041 |
| 200 | 0.112 |
| 201 | 0.067 |
| 202 | 0.023 |
| 203 | 0.020 |
| 204 | 0.041 |
| 205 | 0.008 |
| 206 | 0.014 |
| 207 | 0.010 |
| 208 | 0.004 |
| 209 | 0.005 |
| 210 | 0.032 |
| 211 | 0.007 |
| 212 | 0.014 |
| 213 | 0.027 |
| 214 | 0.010 |
| 242 | 0.008 |
| 243 | 0.003 |
| 244 | 0.003 |
| 245 | 0.096 |
| 246 | 0.041 |
| 247 | 0.245 |
| 248 | 0.003 |
| 215 | 0.765 |
| 216 | 1.207 |
| 217 | 0.839 |
| 218 | 1.704 |
| 219 | 1.192 |
| 220 | 0.904 |
| 221 | 1.599 |
| 222 | 0.991 |
| 223 | 0.097 |
| 224 | 0.924 |
| 225 | 0.444 |
| 226 | 0.582 |
| 227 | 0.191 |
| 228 | 0.319 |
| 229 | 0.646 |
| 230 | 0.030 |
| 231 | 0.057 |
| 232 | 0.028 |
| 233 | 0.026 |
| 234 | 0.071 |
| 235 | 0.041 |
| 236 | 0.045 |
| 237 | 0.043 |
| 238 | 0.035 |
| 239 | 0.089 |
| 240 | 0.030 |
| 241 | 0.124 |
| 249 | 0.0643 |
| 252 | 0.009 |
| 252 | 0.008 |
| 253 | 0.177 |
| 254 | 0.367 |
| 255 | 0.053 |
| 256 | 0.043 |
| 257 | 0.033 |
| 258 | 0.0026 |
| 259 | 0.0026 |
| 260 | 0.0023 |
| 261 | 0.007 |
| 262 | 0.0077 |
| 263 | 0.0031 |
| 264 | 0.058 |
| 265 | 0.283 |
| 266 | 0.015 |
| 267 | 0.025 |
| 268 | 0.049 |
| 269 | 0.011 |
| 270 | 1.074 |

The Potentiation Factor (PF₅₀) for compounds is calculated as a ratio of the IC₅₀ of control cell growth divided by the IC₅₀ of cell growth+PARP inhibitor. Growth inhibition curves for both control and compound treated cells are in the presence of the alkylating agent methyl methanesulfonate (MMS). The test compounds were used at a fixed concentration of 0.2 or 0.5 micromolar. The concentrations of MMS were over a range from 0 to 10 μg/ml.

Cell growth was assessed using the sulforhodamine B (SRB) assay (Skehan, P., et al., (1990) New calorimetric cytotoxicity assay for anticancer-drug screening. J. Natl. Cancer Inst. 82, 1107-1112). 2,000 HeLa cells were seeded into each well of a flat-bottomed 96-well microtiter plate in a volume of 100 μL and incubated for 6 hours at 37° C. Cells were either replaced with media alone or with media containing PARP inhibitor at a final concentration of 0.5, 1 or 5 μM. Cells were allowed to grow for a further 1 hour before the addition of MMS at a range of concentrations (typically 0, 1, 2, 3, 5, 7 and 10 μg/ml) to either untreated cells or PARP inhibitor treated cells. Cells treated with PARP inhibitor alone were used to assess the growth inhibition by the PARP inhibitor.

Cells were left for a further 16 hours before replacing the media and allowing the cells to grow for a further 72 hours at 37° C. The media was then removed and the cells fixed with 100 μL of ice cold 10% (w/v) trichloroacetic acid. The plates were incubated at 4° C. for 20 minutes and then washed four times with water. Each well of cells was then stained with 100 μL of 0.4% (w/v) SRB in 1% acetic acid for 20 minutes before washing four times with 1% acetic acid. Plates were then dried for 2 hours at room temperature. The dye from the stained cells was solubilized by the addition of 100 μL of 10 mM Tris Base into each well. Plates were gently shaken and left at room temperature for 30 minutes before measuring the optical density at 564 nM on a Microquant microtiter plate reader.

The following compounds had a mean PF₅₀ of greater than 2 at 200 nM: 242, 243, 244, 248, 252, 257, 258, 259, 260, 261, 263.

All publications referred to above are herein incorporated by reference.

1. A compound of the formula (I): 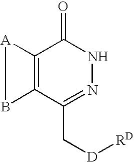
wherein:
A and B together represent an optionally substituted, fused aromatic ring or an optionally substituted, fused cyclohexene ring;
D is selected from: 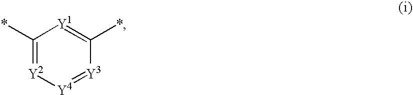
where Y¹ is selected from CH and N, Y² is selected from CH and N, Y³ is selected from CH, CF and N and Y⁴ is selected from CH and N; 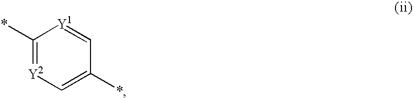
where Y¹ is selected from CH and N and Y² is selected from CH and N; 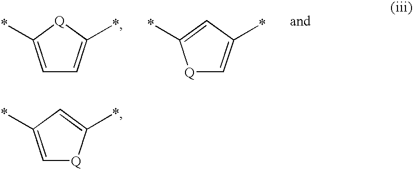
where Q is O or S; and 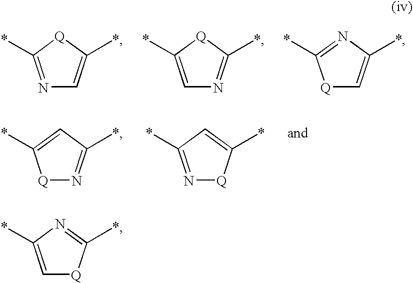
where Q is O or S; and
R^{D} is an optionally substituted C₅₋₂₀ aryl group, bound to D by a carbon-carbon bond.

2. A compound according to claim 1, wherein the fused aromatic ring(s) represented by -A-B- consists solely of carbon ring atoms.

3. A compound according to claim 2, wherein the fused aromatic ring(s) represented by -A-B- is benzene.

4. A compound according to claim 1, wherein -A-B- represent a fused cyclohexene ring, which may bear a single substituent.

5. A compound according to claim 1, wherein D is selected from (i) 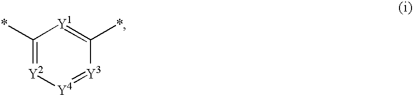
where Y¹ is selected from CH and N, Y² is selected from CH and N, Y³ is selected from CH, CF and N, Y⁴ is selected from CH and N but where only one of Y¹, Y², Y³ and Y⁴ may be N; and 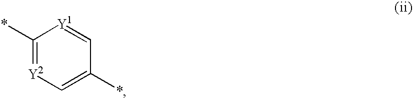
where Y¹ is selected from CH and N and Y² is selected from CH and N, but where only one of Y¹ and Y² may be N.

6. A compound according to claim 5, wherein D is selected from:

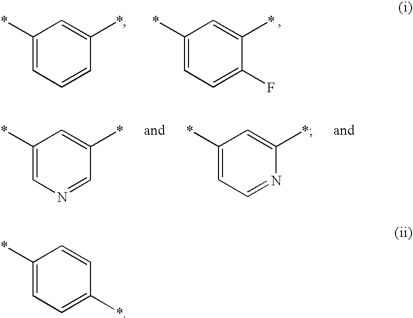

7. A compound according to claim 1, wherein D is selected from: 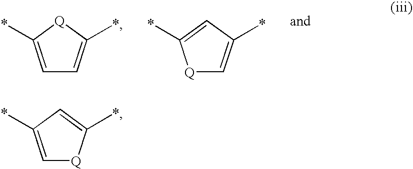
where Q is S; and 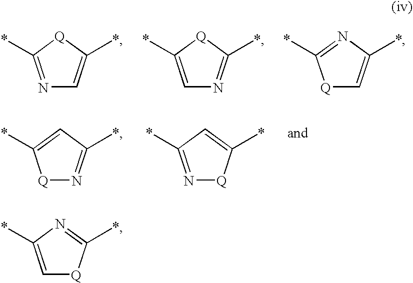
where Q is S.

8. A compound according to claim 7, wherein D is selected from:

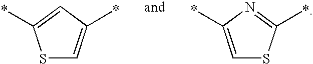

9. A compound according to claim 1, wherein R^{D} is substituted by a group selected from: C₁₋₂₀ alkyl, C₃₋₂₀ heterocyclyl, C₅₋₂₀ aryl, ester, ether, cyano, acyl, acylamido, halo, nitro, dioxyalkylene, oxyalkylene, amido, sulfonyl, thioether, amino, sulfonamino, ureido, carboxy and hydroxyl.

10. A compound according to claim 1, wherein R^{D} is selected from furanyl, thiophenyl, pyrrolyl, pyridyl, pyrazinyl, pyrimidinyl, benzothiophenyl, indolyl, quinolinyl, isoquinolinyl, dibenzofuranyl, dibenzothiophenyl, thianthrenyl and phenoxathiinyl.

11. A compound according to claim 1, wherein R^{D} is selected from phenyl and napthyl.

12. A compound according to claim 11, wherein R^{D} is phenyl, and is either unsubstituted or substituted by one or two groups selected from C₁₋₂₀ alkyl, C₅₋₂₀ aryl, ester, ether, cyano, acyl, acylamido, halo, nitro, dioxyalkylene, oxyalkylene, amido, sulfonyl, thioether, amino, sulfonamino, ureido, carboxy and hydroxyl.

13. A pharmaceutical composition comprising a compound according to claim 1 and a pharmaceutically acceptable carrier or diluent.

14. A method of treating a disease ameliorated by the inhibition of PARP, comprising administering to a subject in need of treatment a therapeutically-effective amount of a compound according to claim 1.

15. A method of treating cancer comprising administering to a subject in need of treatment a therapeutically-effective amount of a compound according to claim 1 either alone or in combination simultaneously or sequentially with radiotherapy or chemotherapeutic agents.
